(19) United States
(12) Patent Application Publication
(10) Pub. No.: US 2020/0115461 A1
(43) Pub. Date: Apr. 16, 2020

(54) COMPOSITIONS AND METHODS FOR ADOPTIVE CELL THERAPIES

(71) Applicant: Harpoon Therapeutics, Inc., South San Francisco, CA (US)

(72) Inventors: Luke EVNIN, San Francisco, CA (US); Holger WESCHE, South San Francisco, CA (US); Kevin WRIGHT, South San Francisco, CA (US)

(21) Appl. No.: 16/610,405

(22) PCT Filed: May 3, 2018
(86) PCT No.: PCT/US2018/030983

§ 371 Date: Nov. 1, 2019

Related U.S. Application Data
(60) Provisional application No. 62/501,007, filed on May 3, 2017.
(60) Provisional application No. 62/500,995, filed on May 3, 2017.

(51) Int. Cl. C07K 16/28; C07K 14/725; A61P 35/00; C07K 14/705

(52) U.S. Cl. CPC C07K 16/2863; A61K 38/00; A61P 35/00; C07K 14/70517; C07K 14/70578; C07K 2319/03; C07K 2319/02; C07K 2317/565; C07K 2319/50; C07K 2317/622; C07K 2317/92; C07K 2317/76; C07K 2317/24; C07K 2319/30; C07K 2319/33; C07K 2317/569; C07K 2317/31; C07K 14/7051

(57) ABSTRACT

Provided herein are immune cells engineered to express one or more cell surface receptor polypeptides containing activatable antigen receptor polypeptides, and methods of use thereof for the treatment of diseases, including cancer.

### CROSS-REFERENCE

This application claims benefit of U.S. Provisional Patent Application No. 62/500,995 filed on May 3, 2017, and U.S. Provisional Patent Application No. 62/501,007 filed on May 3, 2017, each incorporated herein by reference in its entirety.

### BACKGROUND

The selective destruction of an individual cell or a specific cell type is often desirable in a variety of clinical settings. For example, it is a primary goal of cancer therapy to specifically destroy tumor cells, while leaving healthy cells and tissues intact and undamaged. One such method is by inducing an immune response against the tumor, to make immune effector cells such as natural killer (NK) cells or cytotoxic T lymphocytes (CTLs) attack and destroy tumor cells. Recent developments using chimeric antigen receptor (CAR) modified autologous T-cell therapy, which relies on redirecting genetically engineered T-cells to a suitable cell-surface molecule on cancer cells, show promising results in harnessing the power of the immune system to treat B cell malignancies (see, e.g., Sadelain et al., Cancer Discovery 3:388-398 (2013)). The clinical results with CD19-specific CAR T-cells (called CTL019) have shown complete remissions in patients suffering from chronic lymphocytic leukemia (CLL) as well as in childhood acute lymphoblastic leukemia (ALL) (see, e.g., Kalos et al., Sci Transl Med 3:95ra73 (2011), Porter et al., NEJM 365:725-733 (2011), Grupp et al., NEJM 368:1509-1518 (2013)). An alternative approach is the use of autologous T-cells that are genetically engineered to express modified T-cell receptor (TCR) alpha and beta chains with specificity towards a tumor-associated peptide antigen. These TCR chains may form complete TCR complexes and with enhance the specificity of T cells towards one or more antigens. Encouraging results were obtained with engineered autologous T-cells expressing NY-ESO-1-specific TCR alpha and beta chains in patients with synovial carcinoma. There is a clear need to improve genetically engineered T-cells to more broadly act against various human malignancies.

### SUMMARY

One embodiment provides an activatable receptor comprising:
(a) an antigen binding domain comprising
   (i) a VH target-binding domain (VH) and an inactive VL domain (iVL) that binds to the VH domain, wherein the VH and iVL are attached via a linker (L1); and
   (ii) a VL target-binding domain (VL) and an inactive VH domain (iVH) that binds to the VL domain, wherein the VL and iVH are attached via a linker (L2);
(b) a transmembrane domain; and
(c) an intracellular signaling domain;
wherein L1 comprises a first protease cleavage site and L2 comprises a second protease cleavage site.

One embodiment provides an activatable receptor comprising:
(a) an antigen binding domain comprising
   (i) a VH target-binding domain (VH) and an inactive VL domain (iVL) that binds to the VH domain, wherein the VH and iVL are attached via a linker (L1); and
   (ii) a VL target-binding domain (VL) and an inactive VH domain (iVH) that binds to the VL domain, wherein the VL and iVH are attached via a linker (L2);
(b) a transmembrane domain; and
(c) an intracellular signaling domain;
wherein iVL comprises a first protease cleavage site and iVH comprises a second protease cleavage site.

In some embodiments, the first protease cleavage site and the second protease cleavage site are located within a CDR1, CDR2, or CDR3. In some embodiments, the transmembrane domain comprises a transmembrane domain of a T-cell receptor, CD28, CD3, CD45, CD4, CD5, CD8, CD9, CD16, CD22, CD33, CD37, CD64, CD80, CD86, CD134, CD137, or CD154, or a portion thereof. In some embodiments, the intracellular signaling domain comprises an intracellular domain of CD3 zeta, FcR gamma, FcR beta, CD3 gamma, CD3 delta, CD3 epsilon, CD5, CD22, CD79a, CD79b, or CD66d, or a portion thereof. In some embodiments, the intracellular signaling domain comprises a costimulatory domain. In some embodiments, the costimulatory domain comprises an intracellular domain of CD27, CD28, 4-1BB, OX40, CD30, CD40, PD1, ICOS, LFA-1, CD2, CD7, LIGHT, NKG2C, B7-H3, GITR, BAFFR, HVEM, SLAMF7, NKp80, or CD160, or a portion thereof.

One embodiment provides an activatable receptor comprising:
(a) an antigen binding domain comprising
   (i) a VH target-binding domain (VH) and an inactive VL domain (iVL) that binds to the VH domain, wherein the VH and iVL are attached via a linker (L1); and
   (ii) a VL target-binding domain (VL) and an inactive VH domain (iVH) that binds to the VL domain, wherein the VL and iVH are attached via a linker (L2); and
(b) a T Cell Receptor subunit or portion thereof;
wherein L1 comprises a first protease cleavage site and L2 comprises a second protease cleavage site.

One embodiment provides an activatable receptor comprising:
(a) an antigen binding domain comprising
   (i) a VH target-binding domain (VH) and an inactive VL domain (iVL) that binds to the VH domain, wherein the VH and iVL are attached via a linker (L1); and
   (ii) a VL target-binding domain (VL) and an inactive VH domain (iVH) that binds to the VL domain, wherein the VL and iVH are attached via a linker (L2); and
(b) a T Cell Receptor subunit or portion thereof;
wherein iVL comprises a first protease cleavage site and iVH comprises a second protease cleavage site.

In some embodiments, the T Cell Receptor subunit or portion thereof comprises a subunit from TCR-alpha, TCR-beta, CD3-gamma, CD3-epsilon, or CD3-delta, or a portion thereof.

One embodiment provides an activatable receptor comprising:
(a) a first polypeptide comprising a VH target-binding domain (VH) and an inactive VL domain (iVL) that binds to the VH domain, wherein the VH and VL are attached via a linker (L1);
(b) a second polypeptide comprising a VL target-binding domain (VL) and an inactive VH domain (iVH) that binds to the VL domain, wherein the VL and iVH are attached via a linker (L2); and
(c) a transmembrane domain; and
(d) an intracellular signaling domain;
wherein L1 comprises a first protease cleavage site and L2 comprises a second protease cleavage site.

One embodiment provides an activatable receptor comprising:
(a) a first polypeptide comprising a VH target-binding domain (VH) and an inactive VL domain (iVL) that binds to the VH domain, wherein the VH and iVL are attached via a linker (L1);
(b) a second polypeptide comprising a VL target-binding domain (VL) and an inactive VH domain (iVH) that binds to the VL domain, wherein the VL and iVH are attached via a linker (L2); and
(c) a transmembrane domain; and
(d) an intracellular signaling domain;
wherein iVL comprises a first protease cleavage site and iVH comprises a second protease cleavage site.

One embodiment provides an activatable receptor comprising:
(a) a first polypeptide comprising (i) a VH target-binding domain (VH), (ii) an inactive VL domain (iVL) that binds to the VH domain, wherein the VH and iVL are attached via a linker (L1), and (iii) a transmembrane domain; and
(b) a second polypeptide comprising (i) a VL target-binding domain (VL), (ii) an inactive VH domain (iVH) that binds to the VL domain, wherein the VL and iVH are attached via a linker (L2), and (iii) a transmembrane domain;
wherein the first polypeptide or the second polypeptide comprises an intracellular signaling domain; and
wherein L1 comprises a first protease cleavage site and L2 comprises a second protease cleavage site.

One embodiment provides an activatable receptor comprising:
(a) a first polypeptide comprising (i) a VH target-binding domain (VH), (ii) an inactive VL domain (iVL) that binds to the VH domain, wherein the VH and iVL are attached via a linker (L1), and (iii) a transmembrane domain; and
(b) a second polypeptide comprising (i) a VL target-binding domain (VL), (ii) an inactive VH domain (iVH) that binds to the VL domain, wherein the VL and iVH are attached via a linker (L2), and (iii) a transmembrane domain;
wherein the first polypeptide or the second polypeptide comprises an intracellular signaling domain; and
wherein iVL comprises a first protease cleavage site and iVH comprises a second protease cleavage site.

One embodiment provides an activatable receptor comprising:
(a) a first polypeptide comprising
   (i) a VH target-binding domain (VH) and an inactive VL domain (iVL) that binds to the VH domain, wherein the VH and iVL are attached via a linker (L1); and
   (ii) a T Cell Receptor subunit or portion thereof; and
(b) a second polypeptide comprising
   (i) a VL target-binding domain (VL) and an inactive VH domain (iVH) that binds to the VL domain, wherein the VL and iVH are attached via a linker (L2); and
   (ii) a T Cell Receptor subunit or portion thereof;
wherein L1 comprises a first protease cleavage site and L2 comprises a second protease cleavage site.

One embodiment provides an activatable receptor comprising:
(a) a first polypeptide comprising
   (i) a VH target-binding domain (VH) and an inactive VL domain (iVL) that binds to the VH domain, wherein the VH and iVL are attached via a linker (L1); and
   (ii) a T Cell Receptor subunit or portion thereof; and
(b) a second polypeptide comprising
   (i) a VL target-binding domain (VL) and an inactive VH domain (iVH) that binds to the VL domain, wherein the VL and iVH are attached via a linker (L2); and
   (ii) a T Cell Receptor subunit or portion thereof
wherein iVL comprises a first protease cleavage site and iVH comprises a second protease cleavage site.

In some embodiments, the first polypeptide comprises a dimerization domain. In some embodiments, the second polypeptide comprises a dimerization domain. In some embodiments, the first protease cleavage site and the second protease cleavage site are capable of being cleaved by the same protease. In some embodiments, the first protease cleavage site and the second protease cleavage site are capable of being cleaved by different proteases. In some embodiments, the first protease cleavage site and the second protease cleavage site have the same sequence. In some embodiments, the first protease cleavage site and the second protease cleavage site have different sequences. In some embodiments, the first protease cleavage site and the second protease cleavage site are capable of being cleaved by at least one of a serine protease, a cysteine protease, an aspartate protease, a threonine protease, a glutamic acid protease, a metalloproteinase, a gelatinase, and a asparagine peptide lyase. In some embodiments, the first protease cleavage site and the second protease cleavage site are capable of being cleaved at the site of a tumor. In some embodiments, the VH, iVL, and L1 form a scFv. In some embodiments, the VL, iVH, and L2 form a scFv. In some embodiments, the iVH and iVL have a reduced binding affinity for an antigen. In some embodiments, the VH and iVL are associated via a salt bridge. In some embodiments, the VL and iVH are associated via a salt bridge.

One embodiment provides an activatable receptor comprising:
(a) an antigen binding domain comprising
   (i) a VH target-binding domain (VH);
   (ii) a VL target-binding domain (VL); and
   (iii) an inactive binding domain attached to VH or VL via a linker (L1);
(b) a transmembrane domain; and
(c) an intracellular signaling domain;
wherein L1 comprises a protease cleavage site.

One embodiment provides an activatable receptor comprising:
(a) an antigen binding domain comprising
   (i) a VH target-binding domain (VH);
   (ii) a VL target-binding domain (VL); and
   (iii) an inactive binding domain attached to VH or VL via a linker (L1);
(b) a transmembrane domain; and
(c) an intracellular signaling domain.
wherein the inactive binding domain comprises a protease cleavage site.

In some embodiments, the protease cleavage site is located within a CDR1, CDR2, or CDR3.

One embodiment provides an activatable receptor comprising:
(a) an antigen binding domain comprising
   (i) a VH target-binding domain (VH);
   (ii) a VL target-binding domain (VL); and
   (iii) an inactive binding domain attached to VH or VL via a linker (L1); and
(b) a T Cell Receptor subunit or portion thereof;
wherein L1 comprises a protease cleavage site.

One embodiment provides an activatable receptor comprising:
(a) an antigen binding domain comprising
   (i) a VH target-binding domain (VH);
   (ii) a VL target-binding domain (VL); and
   (iii) an inactive binding domain attached to VH or VL via a linker (L1); and
(b) a T Cell Receptor subunit or portion thereof;
wherein the inactive binding domain comprises a protease cleavage site.

In some embodiments, upon activation, VH and VL associate to form an active target-binding domain. One embodiment provides an activatable receptor comprising:
(a) an antigen binding domain;
(b) an inhibitory domain that prevents binding of the target antigen binding domain to a target;
(c) a transmembrane domain; and
(d) an intracellular signaling domain
wherein the antigen binding domain and the inhibitory domain are attached via a linker comprising a protease cleavage site.

One embodiment provides an activatable receptor comprising:
(a) an antigen binding domain;
(b) an inhibitory domain that prevents binding of the target antigen binding domain to a target; and
(c) a T Cell Receptor subunit or portion thereof;
wherein the antigen binding domain and the inhibitory domain are attached via a linker comprising a protease cleavage site.

In some embodiments, the inhibitory domain is a sdAb, an inactive VHH domain, a peptide, or a ligand. In some embodiments, the protease cleavage site is capable of being cleaved by at least one of a serine protease, a cysteine protease, an aspartate protease, a threonine protease, a glutamic acid protease, a metalloproteinase, a gelatinase, and a asparagine peptide lyase. In some embodiments, the protease cleavage site is capable of being cleaved at the site of a tumor. In some embodiments, upon activation, the activatable receptor binds to a tumor antigen. In some embodiments, the tumor antigen is CD 19, CD 123, CD22, CD30, CD 171, CS-1, CLL-1 (CLECL1), CD33, CD161, CD71, EGFRvDI, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC 1, EGFR, NCAM, Prosiase, PAP, ELF2M, Ephrin B2, 1GF-1 receptor, CA1X, LMP2, g 100, bcr-abl, tyrosinase, EphA2, Fucosyl GM1, sLe, GM3, TGS5, HMWMAA, o-acetyl-GD2, Folate receptor beta, TEM1/CD248, TEM7R, CLD 6, TSHR, GPRC5D, CXORF61, CD97, CD 179a, ALK, Poiysialic acid, PLAC 1, GloboH, NY-BR-1, UPK2, HAVCR1. ADRB3, PANX3, GPR20, LY6K, OR51 E2, TARP, WT1, NY-ESO-1, LAGE-1a, legumain, HPV E6,E7, MAGE-A1, ETV6-AML, sperm protein 17, XAGE1, Tie 2, MAD-CT-1, MAD-CT-2, Fos-related antigen 1, p53, p53 mutant, prostein, survivin and telornerase, PCTA-1/Galectin 8, MelanA/MART1, Ras mutant, hTERT, sarcoma translocation breakpoints, ML-IAP, ERG (TMPRSS2 ETS fusion gene), NA 17, PAX3, Androgen receptor, Cyclin B 1, MYCN, RhoC, TRP-2, CYP 1 B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC 12A, BST2, EMR2, LY75, GPC3, FCRL5, AXL, IGF-1R, CD25, CD49C, gpA33, MUC-16, or IGLL1. In some embodiments, upon activation, the activatable receptor binds to a tumor-supporting antigen. In some embodiments, the tumor-supporting antigen is stromal cell antigen. In some embodiments, the stromal cell antigen is bone marrow stromal cell antigen 2 (BST2), fibroblast activation protein (FAP) or tenascin. In some embodiments, the tumor-supporting antigen is a myeloid-derived suppressor cell (MDSC) antigen. In some embodiments, the MDSC antigen is CD33, CD 11b, CD14, CD 15, or CD66b. In some embodiments, the activatable receptor further comprises a T-cell receptor binding domain, a CD3 binding domain, a CD4 binding domain, or a CD8 binding domain. One embodiment provides an engineered cell comprising the activatable receptor of any of the above embodiments. One embodiment provides a pharmaceutical composition comprising the activatable receptor of any of the above embodiments. One embodiment provides a nucleic acid encoding the activatable receptor of any of any of the above embodiments. One embodiment provides a viral vector comprising the nucleic acid of any of the above embodiments.

One embodiment provides an engineered immune cell comprising an activatable cell surface receptor polypeptide comprising:
(i) an extracellular antigen-recognition polypeptide, wherein the extracellular antigen-recognition polypeptide comprises at least a first binding pair and a second binding pair, wherein:
   a. the first binding pair comprises a V_{L} domain, a first linker domain, and a first stabilizing domain, wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
   b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
(ii) a transmembrane domain; and
(iii) an intracellular signaling domain.

One embodiment provides an engineered immune cell comprising an activatable cell surface receptor polypeptide comprising:
(i) an extracellular antigen-recognition polypeptide, wherein the extracellular antigen-recognition polypeptide comprises at least a binding pair, and a V_{H} domain, wherein:
   a. the binding pair comprises a V_{L} domain, a first linker domain, and a stabilizing domain, wherein the first linker domain is covalently linked to the V_{L} domain and the stabilizing domain, wherein the V_{L} domain and the stabilizing domain are non-covalently associated, and wherein the first linker domain or the stabilizing domain comprises a first protease cleavage site, and
   wherein the stabilizing domain and the V_{H} domain may be covalently associated by a second linker domain comprising a second protease cleavage site;
(ii) a transmembrane domain; and
(iii) an intracellular signaling domain.

One embodiment provides an engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain, wherein the first linker domain is covalently linked to the first V_{L} domain and the first stabilizing domain, wherein the first V_{L} domain and the first stabilizing domain are non-covalently associated, and wherein the first linker domain and/or the first stabilizing domain include a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
wherein the extracellular antigen-recognition polypeptide is covalently connected to a T Cell Receptor subunit binding domain or a CD4 binding domain or a CD8 binding domain, wherein binding of the T Cell Receptor subunit binding domain or a CD4 binding domain or a CD8 binding domain does not substantially induce anergy of the engineered immune cell;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

One embodiment provides an engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain and/or the first stabilizing domain include a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain and/or the second stabilizing domain include a second protease cleavage site;
wherein the extracellular antigen-recognition polypeptide is covalently connected to a T Cell Receptor subunit binding domain or a CD4 binding domain or a CD8 binding domain, wherein binding of the T Cell Receptor subunit binding domain or a CD4 binding domain or a CD8 binding domain does not substantially activate a T Cell Receptor present on the engineered immune cell;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

One embodiment provides an engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
wherein the extracellular antigen-recognition polypeptide is covalently connected to a T Cell Polypeptide binding domain;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

One embodiment provides an engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
wherein the extracellular antigen-recognition polypeptide is covalently connected to a T Cell Receptor-Associated Polypeptide binding domain;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

One embodiment provides an engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain include a second protease cleavage site;
wherein the extracellular antigen-recognition polypeptide is covalently connected to a first dimerization domain; and
wherein the extracellular antigen-recognition polypeptide is covalently connected to a second dimerization domain, wherein the second dimerization domain is covalently connected to a T Cell Receptor subunit or a portion thereof selected from the group consisting of TCR-alpha, TCR-beta, CD3-gamma, CD3-epsilon, and CD3-delta or CD4 or CD8.

One embodiment provides an engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{H} domain, a first linker domain, and a first stabilizing domain wherein the first linker domain is covalently linked to the first V_{H} domain and the first stabilizing domain, wherein the first V_{H} domain and the first stabilizing domain are non-covalently associated, and wherein the first linker domain or the first stabilizing domain includes a first protease cleavage site, and
b. the second binding pair comprising a V_{L} domain, a second linker domain, and a second stabilizing domain wherein the second linker domain is covalently linked to the V_{L} domain and the second stabilizing domain, wherein the V_{L} domain and the second stabilizing domain are non-covalently associated, and wherein the second linker domain or the second stabilizing domain includes a second protease cleavage site;
wherein the extracellular antigen-recognition polypeptide is covalently connected to a first dimerization domain; and
wherein the extracellular antigen-recognition polypeptide is covalently connected to a second dimerization domain, wherein the second dimerization domain is covalently connected to a T Cell Receptor subunit or a portion thereof selected from the group consisting of TCR-alpha, TCR-beta, CD3-gamma, CD3-epsilon, and CD3-delta or CD4 or CD8.

One embodiment provides an engineered immune cell comprising an activatable cell surface receptor polypeptide comprising:
(i) an extracellular antigen-recognition polypeptide, wherein the extracellular antigen-recognition polypeptide comprises at least a first binding pair and a second binding pair, wherein:
   a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
   b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain include a second protease cleavage site;
(ii) a transmembrane domain; and
(iii) an intracellular signaling domain.

One embodiment provides an engineered immune cell comprising an activatable cell surface receptor polypeptide comprising:
(i) an extracellular antigen-recognition polypeptide, wherein the extracellular antigen-recognition polypeptide comprises at least a binding pair, and a V_{L} domain, wherein:
   a. the binding pair comprises a V_{H} domain, a first linker domain, and an stabilizing domain, wherein the first linker domain is covalently linked to the V_{H} domain and the stabilizing domain, wherein the V_{H} domain and the stabilizing domain are non-covalently associated, and wherein the first linker domain or the stabilizing domain comprises a first protease cleavage site, and
   wherein the stabilizing domain and the V_{L} domain may be covalently associated by a second linker domain comprising a second protease cleavage site;
(ii) a transmembrane domain; and
(iii) an intracellular signaling domain.

One embodiment provides an engineered immune cell comprising a cell surface receptor polypeptide comprising:
(i) an extracellular antigen-recognition polypeptide, wherein the extracellular antigen-recognition polypeptide comprises at least a first binding pair and a second binding pair, wherein:
   a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
   b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
(ii) wherein the extracellular antigen-recognition polypeptide is covalently connected to a T Cell antigen binding domain, wherein the T Cell antigen binding domain is capable of binding substantially specific to T cells; and
(iii) wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

One embodiment provides an engineered immune cell comprising a cell surface receptor polypeptide comprising:
(i) an extracellular antigen-recognition polypeptide, wherein the extracellular antigen-recognition polypeptide comprises at least a first binding pair and a second binding pair, wherein:
   a. the first binding pair comprises a first V_{L} domain, a first linker domain comprising a first protease cleavage site, and an inactive first V_{H} domain wherein the first linker domain is covalently linked to the first V_{L} domain and the inactive first V_{H} domain, wherein the first V_{L}domain and the inactive first V_{H} domain are non-covalently associated, and
   b. the second binding pair comprising a second V_{H} domain, a second linker domain comprising a second protease cleavage site, and an inactive second V_{L} domain wherein the second linker domain is covalently linked to the second V_{H} domain and the inactive second V_{L} domain, wherein the second V_{H} domain and the inactive second V_{L} domain are non-covalently associated,
   wherein the inactive first V_{H} domain and the inactive second V_{L} domain independently have a reduced binding affinity for an antigen;
   wherein the inactive first V_{H} domain and the inactive second V_{L} domain are covalently associated by a third linker domain comprising a third protease cleavage site;
(ii) a transmembrane domain; and
(iii) an intracellular signaling domain.

One embodiment provides an engineered immune cell comprising a cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a first V_{L} domain, a first linker domain comprising a first protease cleavage site, and an inactive first V_{H} domain wherein the first linker domain is covalently linked to the first V_{L} domain and the inactive first V_{H} domain, wherein the first V_{L} domain and the inactive first V_{H} domain are non-covalently associated, and
b. the second binding pair comprising a second V_{H} domain, a second linker domain comprising a second protease cleavage site, and an inactive second V_{L} domain wherein the second linker domain is covalently linked to the second V_{H} domain and the inactive second V_{L} domain, wherein the second V_{H} domain and the inactive second V_{L} domain are non-covalently associated,
wherein the inactive first V_{H} domain and the inactive second V_{L} domain independently have a reduced binding affinity for an antigen;
wherein the inactive first V_{H} domain and the inactive second V_{L} domain are covalently associated by a third linker domain comprising a third protease cleavage site;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

One embodiment provides an engineered immune cell comprising a population of cell surface receptor polypeptides comprising:
(i) a first cell surface receptor polypeptide comprising:
   a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
   b. a first dimerization domain;
   c. a first transmembrane domain;
   d. a first intracellular signaling domain; and
(ii) a second cell surface receptor polypeptide comprising:
   a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{L} domain and the second stabilizing domain, wherein the V_{L} domain and the second stabilizing domain are non-covalently associated, and wherein the second linker domain or the second stabilizing domain includes a second protease cleavage site;
   b. a second dimerization domain which is optionally identical to the first dimerization domain;
   c. a second transmembrane domain; and
   d. a second intracellular signaling domain.

One embodiment provides an engineered immune cell comprising a population of cell surface receptor polypeptides comprising:
(i) a first cell surface receptor polypeptide comprising:
   a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
   b. a first transmembrane domain; and
   c. a first intracellular signaling domain; and
(ii) a second cell surface receptor polypeptide comprising:
   a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{L} domain and the second stabilizing domain, wherein the V_{L} domain and the second stabilizing domain are non-covalently associated, and wherein the second linker domain or the second stabilizing domain includes a second protease cleavage site;
   b. a second transmembrane domain; and
   c. a second intracellular signaling domain.

One embodiment provides an engineered immune cell comprising a population of cell surface receptor polypeptides comprising:
(i) a first cell surface receptor polypeptide comprising:
   a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
   b. a first dimerization domain;
   c. a T Cell Receptor subunit or portion thereof selected from the group consisting of TCR-alpha, TCR-beta, CD3-gamma, CD3-epsilon, and CD3-delta or CD4 or CD8; and,
(ii) a second cell surface receptor polypeptide comprising:
   a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain include a second protease cleavage site;
   b. a second dimerization domain which is optionally identical to the first dimerization domain;
   c. a T Cell Receptor subunit or portion thereof selected from the group consisting of TCR-alpha, TCR-beta, CD3-gamma, CD3-epsilon, and CD3-delta, or CD4 or CD8.

One embodiment provides an engineered immune cell comprising a population of cell surface receptor polypeptides comprising:
(i) a first cell surface receptor polypeptide comprising:
   a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
   b. a T Cell Receptor subunit or portion thereof selected from the group consisting of TCR-alpha, TCR-beta, CD3-gamma, CD3-epsilon, and CD3-delta or CD4 or CD8; and,
(ii) a second cell surface receptor polypeptide comprising:
   a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
   b. a T Cell Receptor subunit or portion thereof selected from the group consisting of TCR-alpha, TCR-beta, CD3-gamma, CD3-epsilon, and CD3-delta, or CD4 or CD8.

In some embodiments, one or more further protease cleavage sites are located within the first stabilization domain, the second stabilization domain, the first linker, the second linker, or the third linker.

One embodiment provides an engineered immune cell comprising an engineered antigen receptor polypeptide or polypeptide complex, wherein the engineered antigen receptor polypeptide or polypeptide complex comprises an extracellular antigen-recognition polypeptide that specifically binds to a peptide or a polypeptide present in a tumor antigen, wherein the extracellular antigen-recognition polypeptide comprises an antigen binding domain, a stabilization domain, and a linker domain, wherein the stabilization domain and/or the linker domain comprise a protease cleavage site.

One embodiment provides an engineered immune cell comprising an engineered antigen receptor polypeptide or polypeptide complex, wherein the engineered antigen receptor polypeptide or polypeptide complex comprises (i) a V_{HH} domain, (ii) a linker domain, (iii) a inhibitory domain, wherein the linker domain is covalently linked to the V_{HH} domain and the inhibitory domain, and wherein the linker domain and/or the inhibitory domain include a protease cleavage site, (iv) a transmembrane domain, and (v) an intracellular signaling domain.

One embodiment provides an engineered immune cell comprising an engineered antigen receptor polypeptide or polypeptide complex, wherein the engineered antigen receptor polypeptide or polypeptide complex comprises: (i) a V_{HH} domain, (ii) a linker domain, (iii) a inhibitory domain, wherein the linker domain is covalently linked to the V_{HH} domain and the inhibitory domain, and wherein the linker domain and/or the inhibitory domain include a protease cleavage site, and (iv) a T Cell Receptor subunit or portion thereof selected from the group consisting of TCR-alpha, TCR-beta, CD3-gamma, CD3-epsilon, and CD3-delta, or CD4 or CD8.

One embodiment provides an engineered immune cell comprising an engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, iii) a first linker domain comprising a first protease cleavage site, wherein the first linker domain is coupled to the first antibody or antigen binding domain and the masking domain, and iv) a T Cell Receptor subunit or portion thereof selected from the group consisting of TCR-alpha, TCR-beta, CD3-gamma, CD3-epsilon, and CD3-delta, or CD4 or CD8.

One embodiment provides an engineered immune cell comprising an engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, iii) a first linker domain comprising a first protease cleavage site, wherein the first linker domain is coupled to the a first antibody or antigen binding domain and the masking domain, iv) a transmembrane domain, and v) an intracellular signaling domain.

One embodiment provides an engineered immune cell comprising an engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, and iii) a first linker domain comprising a first protease cleavage site, wherein the first linker domain is coupled to the a first antibody or antigen binding domain and the masking domain.

One embodiment provides an engineered immune cell comprising a cell surface receptor comprising:
(i) an extracellular antigen-recognition polypeptide that targets a tumor antigen, wherein the extracellular antigen-recognition polypeptide comprises a first domain comprising an activatable binding domain, a second domain comprising an inactive binding domain, and a first linker domain comprising a first protease cleavage site, wherein the first domain and the second domain are non-covalently associated whereby the second domain prevents binding of the first domain to the tumor antigen, and wherein the second domain is released from the first domain upon proteolytic cleavage at the first protease cleavage site;
(ii) a transmembrane domain; and
(iii) an intracellular signaling domain.

One embodiment provides an engineered immune cell comprising a T Cell Receptor polypeptide comprising:
(i) an extracellular antigen-recognition polypeptide comprising a single chain variable fragment (scFv) domain that immunospecifically binds a tumor antigen, wherein the scFv domain comprises a first V_{H} domain, a first V_{L} domain, and at least one inactive binding domain covalently associated with the first V_{H} domain or the first V_{L} domain, via a linker, and non-covalently associated with the first V_{H} domain or the first V_{L} domain, wherein the at least one inactive binding domain comprises a first protease cleavage site;
(ii) a transmembrane domain; and
(iii) an intracellular signaling domain,
wherein the T Cell Receptor polypeptide is capable of functionally interacting with a T Cell Receptor subunit selected from TCR-alpha, TCR-beta, CD3-gamma, CD3-epsilon, and CD3-delta, or CD4 or CD8.

One embodiment provides an engineered immune cell comprising an engineered antigen receptor polypeptide or polypeptide complex, wherein the engineered antigen receptor polypeptide or polypeptide complex comprises an extracellular antigen-recognition polypeptide comprising:
i) a first antigen binding domain,
ii) a first linker domain comprising a first protease cleavage site,
iii) a second antigen binding domain,
iv) a second linker domain comprising a second protease cleavage site,
v) a third antigen binding domain,
vi) a third linker domain comprising a third protease cleavage site,
vii) a fourth antigen binding domain,
viii) a fifth antigen binding domain capable of binding a T cell, a TCR subunit or a CD3 delta subunit,
ix) a CD3 epsilon transmembrane domain; and
x) a CD3 epsilon intracellular signaling domain;
wherein:
   c. the first linker domain is located between the first antigen binding domain and the second antigen binding domain;
   d. the second linker domain is located between the second antigen binding domain and the third antigen binding domain;
   e. the third linker domain is located between the third antigen binding domain and the fourth antigen binding domain; and
   wherein the engineered antigen receptor polypeptide or polypeptide complex is capable of functionally associating with a T Cell Receptor complex or at least one T Cell Receptor (TCR) subunit.

One embodiment provides an engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, and iii) a first linker domain comprising a first protease cleavage site, wherein the first linker domain is coupled to the a first antibody or antigen binding domain and the masking domain.

One embodiment provides an engineered immune cell comprising an engineered antigen receptor polypeptide or polypeptide complex, wherein the engineered antigen receptor polypeptide or polypeptide complex comprises
i) an extracellular antigen-recognition polypeptide that specifically binds to a peptide or a polypeptide present in a tumor antigen, wherein the extracellular antigen-recognition polypeptide comprises a first antigen binding domain, optionally a second antigen binding domain,
ii) a blocking polypeptide,
iii) a first linker domain comprising a first protease cleavage site, wherein the first linker domain is located between the extracellular antigen-recognition polypeptide and the blocking polypeptide;
iv) a transmembrane domain; and
v) an intracellular domain;
wherein the engineered antigen receptor polypeptide or polypeptide complex is capable of functionally associating with a T Cell Receptor (TCR) subunit.

In some embodiments, the extracellular antigen-recognition polypeptide, upon cleavage of one of the protease cleavage sites has an increased binding affinity for a tumor antigen. In some embodiments, the first stabilizing domain and/or the second stabilizing domain becomes dissociated from any polypeptide with which it was non-covalently associated upon cleavage of one of the protease cleavage sites contained in the first stabilizing domain and/or in the second stabilizing domain. In some embodiments, the first stabilizing domain and/or the second stabilizing domain becomes dissociated from any polypeptide from which it was non-covalently associated upon cleavage of one of the protease cleavage sites in the linker domain, in the first stabilizing domain, and/or in the second stabilizing domain. In some embodiments, the first stabilizing domain reduces the target binding of an antigen-recognition polypeptide to which it is non-covalently associated, upon cleavage of one of the protease cleavage sites. In some embodiments, the first stabilizing domain and the second stabilizing domain are covalently associated by a third linker domain comprising a third protease cleavage site. In some embodiments, the first stabilizing domain and either the first V_{H} domain or the second V_{L} domain is covalently associated by a third linker domain comprising a third protease cleavage site. In some embodiments, the polypeptide comprises (i) extracellular antigen-recognition polypeptide, the transmembrane domain, and the intracellular signaling domain are derived from CD3-epsilon and (ii) the extracellular T Cell Receptor (TCR) subunit recognition polypeptide immunospecifically binds to CD3-delta. In some embodiments, the polypeptide comprises (i) the extracellular antigen-recognition polypeptide, the transmembrane domain, and the intracellular signaling domain are derived from TCR-alpha and (ii) the extracellular T Cell Receptor (TCR) subunit recognition polypeptide immunospecifically binds to TCR-beta. In some embodiments, the T Cell Receptor (TCR) subunit recognition domain comprises an scFv domain or a single domain antibody. In some embodiments, the first protease cleavage site and the second protease cleavage site are susceptible to a first protease. In some embodiments, the first protease cleavage site is susceptible to a tumor-associated protease. In some embodiments, the extracellular antigen-recognition polypeptide immunospecifically binds to CD 19, CD 123, CD22, CD30, CD 171, CS-1, CLL-1 (CLECL1), CD33, CD166, CD71, EGFRvDI, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC 1, EGFR, NCAM, Prosiase, PAP, ELF2M, Ephrin B2, 1GF-1 receptor, CA1X, LMP2, g 100, bcr-abl, tyrosinase, EphA2, Fucosyl GM1, sLe, GM3, TGS5, HMWMAA, o-acetyl-GD2, Folate receptor beta, TEM1/CD248, TEM7R, CLD 6, TSHR, GPRC5D, CXORF61, CD97, CD 179a, ALK, Poiysialic acid, PLAC 1, GloboH, NY-BR-1, UPK2, HAVCR1. ADRB3, PANX3, GPR20, LY6K, OR51 E2, TARP, WT1, NY-ESO-1, LAGE-1a, legumain, HPV E6, E7, MAGE-A1, ETV6-AML, sperm protein 17, XAGE1, Tie 2, MAD-CT-1, MAD-CT-2, Fos-related antigen 1, p53, p53 mutant, prostein, survivin and telornerase, PCTA-1/Galectin 8, MelanA/MART1, Ras mutant, hTERT, sarcoma translocation breakpoints, ML-IAP, ERG (TMPRSS2 ETS fusion gene), NA 17, PAX3, Androgen receptor, Cyclin B 1, MYCN, RhoC, TRP-2, CYP 1 B 1, BORIS, SART3, PAX5, OY-TES 1, LCK, AKAP-4, SSX2, RAGE-1, human telornerase reverse transcriptase, RU1, RU2, intestinal carboxyl esterase, mut hsp70-2, CD79a, CD79b, CD72, LAIR1, FCAR, LILRA2, CD300LF, CLEC 12A, BST2, EMR2, LY75, GPC3, FCRL5, AXL, IGF-1R, CD25, CD49C, gpA33, MUC-16, or IGLL1. In some embodiments, the extracellular antigen-recognition polypeptide immunospecifically binds to a stromal cell antigen selected from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD 11b, C I 4, CD 15, and CD66b. Accordingly, in some embodiments, the tumor-supporting antigen is chosen from one or more of: bone marrow stromal cell antigen 2 (BST2), fibroblast activation protem (FAP) or tenascin, CD33, CD 11b, C14, CD 15, and CD66b. In some embodiments, the first protease cleavage site is susceptible to a first tumor-associated protease, and wherein the second protease cleavage site is susceptible to a second tumor-associated protease, wherein the first and second tumor-associated protease are not the same protease.

In some embodiments, the extracellular antigen-recognition polypeptide is activated to target a tumor antigen upon: proteolytic cleavage of the first protease cleavage site by a first tumor associated protease, proteolytic cleavage of the second protease cleavage site by a second tumor associated protease, and proteolytic cleavage of the third protease cleavage site by a first serum protease. In some embodiments, the third protease cleavage site is susceptible to a protease present in serum. In some embodiments, the transmembrane domain and/or the intracellular signaling domain comprise a CD3 subunit sequence. In some embodiments, the first domain and the second domain are non-covalently associated whereby the second domain prevents binding of the first domain to the tumor antigen, and wherein the second domain is released from the first domain upon proteolytic cleavage at the first protease cleavage site.

One embodiment provides an engineered immune cell comprising an activatable cell surface receptor polypeptide comprising:
(i) an extracellular antigen-recognition polypeptide, wherein the extracellular antigen-recognition polypeptide comprises at least a first binding pair and a second binding pair, wherein:
   a. the first binding pair comprises a V_{L} domain, a first linker domain, and a first V_{H} domain, wherein the first linker domain is covalently linked to the first V_{L} domain and the first V_{H} domain, wherein the first V_{L} domain and the first V_{H} domain are non-covalently associated, and wherein the first linker domain or the first stabilizing domain includes a first protease cleavage site, and
   b. the second binding pair comprising a second V_{H} domain, a second linker domain, and a second V_{L} domain wherein the second linker domain is covalently linked to the second V_{H} domain and the second V_{L} domain, wherein the second V_{H} domain and the second V_{L} domain are non-covalently associated, and wherein the second linker domain or the second stabilizing domain includes a second protease cleavage site;
(ii) a transmembrane domain; and
(iii) an intracellular signaling domain,
   wherein at least one of the binding pairs recognizes and binds to a chemokine receptor protein prior to cleavage at the protease cleavage site.

A pharmaceutical composition of any one of the preceding embodiments, wherein the engineered antigen receptor polypeptide or polypeptide complex comprises a chimeric antigen receptor (CAR), T cell receptor (TCR) subunit, or a functional non-TCR antigen recognition receptor. A pharmaceutical composition of any one of the preceding embodiments, wherein the first protease cleavage site is susceptible to a tumor-associated protease. A pharmaceutical composition of any one of the preceding embodiments, wherein the engineered antigen receptor polypeptide complex comprises a multispecific antibody. A pharmaceutical composition of any one of the preceding embodiments, wherein the engineered antigen receptor polypeptide complex comprises a single chain variable fragment (scFv) polypeptide capable of being directed to a target antigen, wherein the scFv polypeptide comprises a first V_{H} domain, a first V_{L} domain, and a first linker domain comprising a first protease cleavage site, wherein the first V_{H} domain or the first V_{L} domain does not specifically bind to the target antigen, wherein the target antigen is optionally present on the surface of a tumor cell. A pharmaceutical composition of any one of the preceding embodiments, wherein the engineered antigen receptor polypeptide complex comprises a single chain variable fragment (scFv) polypeptide capable of being directed to a target antigen, wherein the scFv polypeptide comprises a first V_{H} domain, a first V_{L} domain, and a first linker domain comprising a first protease cleavage site, wherein the first V_{H} domain and the first V_{L} domain functionally interact, and wherein the first V_{H} domain or the first V_{L} domain does not specifically bind to the target antigen. A pharmaceutical composition of any one of the preceding embodiments, wherein the engineered antigen receptor polypeptide complex comprises a single chain variable fragment (scFv) polypeptide capable of being directed to a target antigen, wherein the scFv polypeptide comprises a first V_{H} domain, a first V_{L} domain, and a first linker domain comprising a first protease cleavage site, wherein the first V_{H} domain and the first V_{L} domain functionally interact, and wherein the first V_{H} domain or the first V_{L} domain is inactive. A pharmaceutical composition of any one of the preceding embodiments, wherein the engineered antigen receptor polypeptide complex comprises a single chain variable fragment (scFv) polypeptide capable of being directed to a target antigen, wherein the scFv polypeptide comprises a first V_{H} domain, a first V_{L} domain, and a first linker domain comprising a first protease cleavage site, wherein the the scFv polypeptide does not specifically bind to the target antigen. A pharmaceutical composition of any one of the preceding embodiments, wherein the engineered antigen receptor polypeptide complex comprises a single chain variable fragment (scFv) polypeptide capable of being directed to a target antigen, wherein the scFv polypeptide comprises a first V_{H} domain, a first V_{L} domain, and a first linker domain comprising a first protease cleavage site, wherein the the scFv polypeptide has an affinity for the target antigen of weaker than about 50 nM. A pharmaceutical composition of any one of the preceding embodiments, wherein the engineered antigen receptor polypeptide complex comprises a single domain (sd) polypeptide capable of being directed to a target antigen, wherein the sd polypeptide comprises a first V_{H} domain and a first V_{L} domain, and a first linker domain comprising a first protease cleavage site, wherein the first V_{H} domain and the first V_{L} domain functionally interact, and wherein the sd polypeptide does not specifically bind to the target antigen, wherein the target antigen is optionally present on the surface of a tumor cell. A pharmaceutical composition of any one of the preceding embodiments, wherein the engineered antigen receptor polypeptide complex comprises i) a first receptor polypeptide comprising a first single chain variable fragment (scFv) polypeptide capable of being directed to a target antigen, wherein the first scFv polypeptide comprises a first V_{H} domain, a first V_{L} domain, and a first linker domain comprising a first protease cleavage site, wherein the first V_{H} domain and the first V_{L} domain functionally interact, wherein the first V_{H} domain or the first V_{L} domain does not specifically bind to the target antigen, and ii) a second receptor polypeptide second single chain variable fragment (scFv) polypeptide capable of being directed to a target antigen, wherein the second scFv polypeptide comprises a second V_{H} domain, a second V_{L} domain, and a second linker domain comprising a second protease cleavage site, wherein the second V_{H} domain and the second V_{L} domain functionally interact, wherein the second V_{H} domain or the second V_{L} domain does not specifically bind to the target antigen wherein the target antigen is optionally present on the surface of a tumor cell. A pharmaceutical composition of any one of the preceding embodiments, wherein the engineered antigen receptor polypeptide complex further comprises an intracellular signaling domain. A pharmaceutical composition of any one of the preceding embodiments, wherein the engineered antigen receptor polypeptide complex further comprises an intracellular signaling domain comprising a signaling domain of a CD3-zeta chain polypeptide and optionally one or more additional costimulatory domains. A pharmaceutical composition of any one of the preceding embodiments, wherein the first receptor polypeptide and/or the second receptor polypeptide comprises an intracellular signaling domain. A pharmaceutical composition of any one of the preceding embodiments, wherein the engineered antigen receptor polypeptide complex further comprises a transmembrane domain. A pharmaceutical composition of any one of the preceding embodiments, wherein the engineered antigen receptor polypeptide complex further comprises an intracellular signaling domain and a transmembrane domain, wherein the transmembrane domain links the extracellular antigen-recognition polypeptide and the intracellular signaling domain. A pharmaceutical composition of any one of the preceding embodiments, wherein the engineered immune cell comprises a first genetic disruption and a second genetic disruption, wherein the first genetic disruption comprises a first disruption element encoding for the engineered antigen receptor polypeptide or polypeptide complex, and wherein the second genetic disruption comprises a second disruption element resulting in altered expression of a target gene in the engineered immune cell. A pharmaceutical composition of any one of the preceding embodiments, wherein the engineered immune cell comprises a T cell. A pharmaceutical composition of any one of the preceding embodiments, wherein the engineered antigen receptor polypeptide comprises a single chain fragment (scFv) polypeptide or a single domain (sd) polypeptide. A pharmaceutical composition of any one of the preceding embodiments, wherein the transmembrane domain comprises a TCR subunit transmembrane domain or portion thereof. A pharmaceutical composition of any one of the preceding embodiments, wherein the transmembrane domain comprises a TCR subunit intracellular signaling domain or portion thereof.

A nucleic acid encoding the engineered antigen receptor polypeptide or polypeptide complex of any one of the preceding embodiments. A nucleic acid encoding a plurality of engineered antigen receptor polypeptides of any one of the preceding embodiments. A composition comprising a plurality of nucleic acids, wherein the plurality of nucleic acids independently encode one or more engineered antigen receptor polypeptides of any one of the preceding embodiments. A viral vector comprising the nucleic acid of any one of the preceding embodiments.

A method of treatment, comprising administering the pharmaceutical composition of any one of the preceding embodiments. to a human subject having cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of an activatable cell surface receptor as described herein;

FIG. 2 shows a diagram of receptors of the present disclosure used in Example 2;

FIG. 3 shows results from experiments described in Example 2;

FIG. 4 shows a diagram of receptors of the present disclosure used in Example 3 and Example 4;

FIG. 5 shows results from experiments described in Example 3;

FIG. 6 shows results from experiments described in Example 4;

FIG. 7 shows a diagram of receptors of the present disclosure used in Example 5 and Example 6;

FIG. 8 shows results from experiments described in Example 5;

FIG. 9 shows results from experiments described in Example 6;

FIG. 10 shows results from experiments described in Example 7;

FIG. 11 shows a diagram of receptors of the present disclosure described in Example 8, Example 9, and Example 10;

FIG. 12 shows results from experiments described in Example 8;

FIG. 13 shows a diagram of receptors of the present disclosure described in Example 9;

FIG. 14 shows results from experiments described in Example 9;

FIG. 15 shows a diagram of receptors of the present disclosure described in Example 10; and

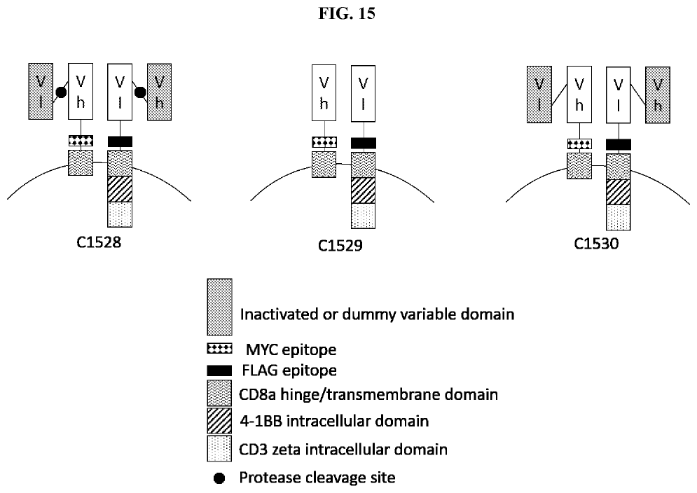

FIG. 16 shows results from experiments described in Example 10.

### DETAILED DESCRIPTION

Overview

In one aspect, described herein are isolated nucleic acid molecules encoding an activatable cell surface receptor polypeptide and immune cells that contain one or more of the activatable cell surface receptor polypeptides or polypeptide complexes. In certain embodiments, the immune cell is a human CD8+ or CD4+ T-cell that comprises at least one activatable cell surface receptor polypeptide or polypeptide complex.

In another aspect, provided herein are methods of generating a population of virally-infected, engineered cells that comprise introducing a virus into a cell, where the virus comprises a nucleic acid encoding any of the described activatable cell surface receptor polypeptides or polypeptide complexes.

In another aspect, provided herein are methods of providing an anti-tumor immunity in a mammal that comprise administering to the mammal an effective amount of a cell expressing any of the described activatable cell surface receptor polypeptides or polypeptide complexes. In some embodiments, the cell is an autologous T-cell. In some embodiments, the cell is an allogeneic T-cell. In some embodiments, the mammal is a human.

In another aspect, provided herein are methods of treating a mammal having a disease associated with expression of a target antigen, such as a tumor antigen, that comprise administering to the mammal an effective amount of the cell of comprising any of the described activatable cell surface receptor polypeptides or polypeptide complexes. In some embodiments, the disease associated with a target antigen expression is selected from a proliferative disease such as a cancer or malignancy or a precancerous condition such as a myelodysplasia, a myelodysplastic syndrome or a preleukemia, or is a non-cancer related indication associated with expression of the target antigen. In some embodiments, the disease is a hematologic cancer selected from the group consisting of one or more acute leukemias including but not limited to B-cell acute lymphoid leukemia (“B-ALL”), T-cell acute lymphoid leukemia (“T-ALL”), acute lymphoblastic leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells.

In some embodiments, the cells expressing any of the described activatable cell surface receptor polypeptides or polypeptide complexes are administered in combination with an agent that ameliorates one or more side effects associated with administration of a cell expressing an activatable cell surface receptor polypeptides or polypeptide complexes. In some embodiments, the cells expressing any of the described activatable cell surface receptor polypeptides or polypeptide complexes are administered in combination with an agent that treats the disease associated with the target antigen.

Also provided herein are any of the described isolated nucleic acid molecules, any of the described isolated polypeptide molecules, any of the described activatable cell surface receptor polypeptides or polypeptide complexes, any of the described vectors or any of the described cells for use as a medicament.

### Definitions

Unless defined otherwise, all technical and scientific terms used herein have the same meaning as commonly understood by one of ordinaiy skill in the art to which the disclosure pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods. Further, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

The term “autologous” refers to any material derived from the same individual to whom it is later to be re-introduced.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be suffi ciently unlike genetically to interact antigenically.

The term “xenogeneic” refers to a graft derived from an animal of a different species. The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The phrase “disease associated with expression of a target antigen” or “disease associated with a tumor antigen” includes, but is not limited to, a disease associated with expression of a tumor antigen as described herein or a condition associated with cells which express a tumor antigen as described herein including, e.g., proliferative diseases such as a cancer or malignancy or a precancerous condition such as a myelodysplasia, a myelodysplasia syndrome or a preleukemia; or a noncancer related indication associated with cells which express a tumor antigen as described herein. In one aspect, a cancer associated with expression of a tumor antigen as described herein is a hematological cancer. In one aspect, a cancer associated with expression of a tumor antigen as described herein is a solid cancer. Further diseases associated with expression of a tumor antigen described herein include, but not limited to, e.g., atypical and/or non-classical cancers, malignancies, precancerous conditions or proliferative diseases associated with expression of a tumor antigen as described herein. Non-cancer related indications associated with expression of a target or tumor antigen as described herein include, but are not limited to, e.g., autoimmune disease, (e.g., lupus), inflammatory disorders (allergy and asthma), infection, viral disease and transplantation.

In some embodiments, the tumor antigen-expressing cells express mRNAs coding for the tumor antigen protein (e.g., wild-type or mutant), and the tumor antigen protein may be present at normal levels or reduced levels. In some embodiments, the tumor antigen-expressing cease expressing mRNAs coding for the tumor antigen protein after a certain period of time. Thus, in some embodiments, the tumor antigen-expressing cells produce detectable levels of a tumor antigen protein at one point, and subsequently produce substantially no detectable tumor antigen protein.

The terms “target antigen”, “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to a target cell, in particular a tumor cell. In some embodiments, a tumor antigen is a marker expressed by both normal cells and cancer cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen is expressed exclusively on the cell surface of a cancer cell, entirely or as a fragment (e.g., MHC/peptide), and not synthesized or expressed on the surface of a normal cell. In some embodiments, the activatable cell surface receptor polypeptides or polypeptide complexes of the present disclosureinclude activatable cell surface receptor polypeptides comprising an antigen binding domain (e.g., antibody or antibody fragment) that binds to a major histocompatibility complex (MHC) presented peptide. Normally, peptides derived from endogenous proteins fill the pockets of MHC class I molecules, and are recognized by T cell receptors (TCRs) on CDS+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A 1 or HLA-A2 have been described (see, e.g., Sastry et a!, J Virol, 2011 85(5); 1935-1942; Sergeeva et al. Blood, 2011 117(16):4262-4272: Verma et al, J Immunol 2010 184(4):2156-2165; Willemsen et al. Gene Ther 2001 8(21): 1601-1608; Dao et al, Sci Transi Med 2013 5(176): 176ra33; Tassev et al. Cancer Gene Ther 2012 19(2): 84-100). For example, a TCR-like antibody can be identified from screening a library, such as a human scFv phage displayed library.

As provided herein, target antigens include tumor-supporting antigens and cancer-supporting antigens. The term ‘tumor-supporting antigen” or “cancer-supporting antigen” interchangeably refer to a molecule (typically a protein, carbohydrate or lipid) that is expressed on the surface of a cell that is, itself, not cancerous, but supports the cancer cells, e.g., by promoting their growth or survival e.g., resistance to immune cells. Exemplar cells of this type include stromal cells and myeloid-derived suppressor cells (MDSCs). The tumor-supporting antigen itself need not play a role in supporting the tumor cells so long as the antigen is present on a cell that supports cancer cells.

As used herein, a “stabilization domain,” a “stabilization V_{H},” and “stabilization V_{L},” refer to an antibody component or portion thereof such as an scFv, which, when paired with their cognate V_{L} (or “active V_{L}”) or V_{H} (or “active V_{H}”) partners, respectively, form a resulting V_{H}/V_{L} pair that stabilizes the cognate V_{L} or V_{H} partners but, generally, does not specifically bind to the antigen (i.e., the stabilization domain is an “inactive” domain) to which the “active” V_{H} or “active” V_{L} would bind were it bound to an analogous V_{L} or V_{H}, which was not “inactive”. Exemplary “stabilization V_{H}” and “stabilization V_{L}” domains are formed by, e.g., mutation of a wild type V_{H} or V_{L} sequence. Exemplary mutations are within CDR1, CDR2 or CDR3 of V_{H} or V_{L}. An exemplary mutation includes placing a domain linker within CDR1, or CDR2 or CDR3, thereby forming a “stabilization V_{H}” or “stabilization V_{L}” domain. Other exemplary “stabilization V_{H}” (or “iVH) and “stabilization V_{L}” (or “iVL”) domains are domains which are not formed by mutation of a wild type sequence, yet have no specificity for the target antigen. For example, an active VH or VL may comprise a sequence which has specificity for a given target antigen, while an iVH or an iVL may comprise a wild type sequence which has no affinity the given target antigen. An “active V_{H}” or “active V_{L}” is one that, upon pairing with its “active” cognate partner, i.e., V_{L} or V_{H}, respectively, is capable of specifically binding to its target antigen.

A “protease cleavage site”, as used herein, comprises polypeptides or polypeptide sequences having a sequence recognized by an enzyme and cleaved in a sequence-specific manner. Antigen binding proteins contemplated herein, in some cases, comprise a protease cleavage domain recognized in a sequence-specific manner by a matrix metalloprotease (MMP), for example MMP9. In some cases, the protease cleavage domain recognized by a MMP9 comprises a polypeptide having an amino acid sequence PR(S/T)(L/I)(S/T). In some cases, the protease cleavage domain recognized by MMP9 comprises a polypeptide having an amino acid sequence LEATA. In some cases, the protease cleavage domain is recognized in a sequence-specific manner by a MMP11. In some cases, the protease cleavage domain recognized by a MMP11 comprises a polypeptide having an amino acid sequence GGAANLVRGG. In some cases, the protease cleavage domain is recognized by a protease disclosed in Table 1. In some cases, the protease cleavage domain recognized by a protease disclosed in Table 1 comprises a polypeptide having an amino acid sequence selected from a sequence disclosed in Table 1.

TABLE 1
Exemplary Proteases and Protease Recognition Sequences

| Protease | Cleavage Domain Sequence | SEQ ID NO: |
|---|---|---|
| MMP7 | KRALGLPG | 25 |
| MMP7 | (DE)₈RPLALWRS(DR)₈ | 26 |
| MMP9 | PR(S/T)(L/I)(S/T) | 27 |
| MMP9 | LEATA | 28 |
| MMP11 | GGAANLVRGG | 29 |
| MMP14 | SGRIGFLRTA | 30 |
| MMP | PLGLAG | 31 |
| MMP | PLGLAX | 32 |
| MMP | PLGC(me)AG | 33 |
| MMP | ESPAYYTA | 34 |
| MMP | RLQLKL | 35 |
| MMP | RLQLKAC | 36 |
| MMP2, MMP9, MMP14 | EP(Cit)G(Hof)YL | 37 |
| Urokinase plasminogen activator (uPA) | SGRSA | 38 |
| Urokinase plasminogen activator (uPA) | DAFK | 39 |
| Urokinase plasminogen activator (uPA) | GGGRR | 40 |
| Lysosomal Enzyme | GFLG | 41 |
| Lysosomal Enzyme | ALAL | 42 |
| Lysosomal Enzyme | FK | 43 |
| Cathepsin B | NLL | 44 |
| Cathepsin D | PIC(Et)FF | 45 |
| Cathepsin K | GGPRGLPG | 46 |
| Prostate Specific Antigen | HSSKLQ | 47 |
| Prostate Specific Antigen | HSSKLQL | 48 |
| Prostate Specific Antigen | HSSKLQEDA | 49 |
| Herpes Simplex Virus Protease | LVLASSSFGY | 50 |
| HIVProtease | GVSQNYPIVG | 51 |
| CMVProtease | GVVQASCRLA | 52 |
| Thrombin | F(Pip)RS | 53 |
| Thrombin | DPRSFL | 54 |
| Thrombin | PPRSFL | 55 |
| Caspase-3 | DEVD | 56 |
| Caspase-3 | DEVDP | 57 |
| Caspase-3 | KGSGDVEG | 58 |
| Interleukin 1β converting enzyme | GWEHDG | 59 |
| Enterokinase | EDDDDKA | 60 |
| FAP | KQEQNPGST | 61 |
| Kallikrein 2 | GKAFRR | 62 |
| Plasmin | DAFK | 63 |
| Plasmin | DVLK | 64 |
| Plasmin | DAFK | 65 |
| TOP | ALLLALL | 66 |

Stabilization domains, generally, include any variant polypeptide. By “variant polypeptide” as used herein is meant a polypeptide sequence that differs from that of a parent polypeptide sequence by virtue of at least one amino acid modification. Modifications can include substitutions, deletions, and additions. Variant polypeptide may refer to the polypeptide itself, a composition comprising the polypeptide, or the amino sequence that encodes it. Preferably, the variant polypeptide has at least one amino acid modification compared to the parent polypeptide, e.g., from about one to about ten amino acid modifications, and preferably from about one to about five amino acid modifications compared to the parent. The variant polypeptide sequence herein will preferably possess at least about 80% homology with a parent polypeptide sequence, most preferably at least about 90% homology, and more preferably at least about 95% homology.

The term “stimulation,” refers to a primary response induced by binding of a stimulatory molecule (e.g., a T Cell Receptor complex) with its cognate ligand (or target antigen such as a tumor antigen in the case of a CAR) thereby mediating a signal transduction event, such as, but not limited to, signal transduction via the TCR/CD3 complex or signal transduction via the appropriate receptor or signaling domains. Stimulation can mediate altered expression of certain molecules.

The term “antigen presenting cell” or “APC” refers to an immune system cell such as an accessory cell (e.g., a B-cell, a dendritic cell, and the like) that displays a foreign antigen complexed with MHCs on its surface. T-cells may recognize these complexes using their T-cell receptors (TCRs). APCs process antigens and present them to T-cells.

An “immune cell” or “Immune effector cell,” as that term is used herein, refers to a cell that is involved in an immune response, e.g., in the promotion of an immune effector response. Examples of immune effector cells include T cells, e.g., alpha/beta T cells and gamma delta T cells, B cells, natural killer (NK) cells, natural killer T (NKT) ceils, mast cells, and myeloic-derived phagocytes.

An “engineered” immune cell includes any modification to an immune cell, such as by recombinant DNA or other genetic engineering or modification, or by the selection of immune cells having desireable functions.

“Immune effector function” or “immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase “nucleotide sequence that encodes a protein” and may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “expression” refers to the transcription and/or translation of a particular nucleotide sequence driven by a promoter.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, S1V, and FIV are examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomelic subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the hum an immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al. Nature, 321: 522-525, 1986; Reichmann et al., Nature, 332: 32.3-329, 1988: Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified fonn, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence, Operably linked DNA sequences can be contiguous with each other and, e.g., where necessary to join two protein coding regions, are in the same reading frame.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. Nucleic Acid Res. 19:5081 (1991); Ohtsuka et al., J. Biol, Cliem. 260:2605-2608 (1985); and Rossolini et al, Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue specific” promoter refers to a nucleotide sequence which, when operably linked with a polynucleotide encodes or specified by a gene, causes the gene product to be produced in a cell substantially only if the cell is a cell of the tissue type corresponding to the promoter.

In the context of the present disclosure, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “flexible polypeptide linker” or “linker” as used in the context of a scFv refers to a peptide linker that consists of amino acids such as glycine and/or serine residues used alone or in combination, to link variable heavy and variable light chain regions together.

As used herein, a 5′cap (also termed an RNA cap, an RNA 7-methylguanosine cap or an RNA m′G cap) is a modified guanine nucleotide that has been added to the “front” or 5′ end of a eukaryotic messenger RNA shortly after the start of transcription. In some embodiments, the 5′ cap consists of a terminal group which is linked to the first transcribed nucleotide. Its presence is critical for recognition by the ribosome and protection from RNases. Cap addition is coupled to transcription, and occurs co-transcriptionaly, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies. In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that has been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., a tumor antigen) protein present in a sample, but which antibody or ligand does not substantially recognize or bind oilier molecules in the sample. “Membrane anchor” or “membrane tethering domain”, as that term is used herein, refers to a polypeptide or moiety, e.g., a myristoyl group, sufficient to anchor an extracellular or intracellular domain to the plasma membrane.

By “extracellular domain” is meant the domain of a transmembrane protein that is expressed outside the cell.

By “membrane protein” is meant a protein that comprises a transmembrane domain and, when expressed in a target cell, is anchored in, or traverses the cell membrane.

By “CD3 delta, gamma, or epsilon domain” is meant a domain that is derived from, and retains at least one endogenous activity of, CD3 delta, CD3 gamma or CD3 epsilon, typically in the context of an intact T Cell Receptor.

The term “signaling domain” refers to the functional portion of a protein which acts by transmitting information within the cell to regulate cellular activity via defined signaling pathways by generating second messengers or functioning as effectors by responding to such messengers.

By “intracellular signaling domain” is meant a functional portion of a signaling domain present within the intracellular region. The intracellular signaling domain can comprise the entire intracellular portion, or the entire native intracellular signaling domain, of the molecule from which it is derived, or a functional fragment or derivative thereof. The intracellular signaling domain generates a signal that promotes an immune effector function of an activatable cell surface receptor polypeptide containing cell, e.g., an activatable cell surface receptor polypeptide-expressing T-cell. Examples of immune effector function include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

By “intracellular co-stimulatory domain” or “costimulatory domain” is meant the intracellular portion of a costimulatory molecule. A costimulatory molecule can be represented in the following protein families: TNF receptor proteins, Immiinoglobulin-like proteins, cytokine receptors, integrins, signaling lymphocytic activation molecules (SLAM proteins), and activating NK cell receptors. Examples of such molecules include CD27, CD28, 4-1BB (CD 137), OX40, GITR, CD30, CD40, ICOS, BAFFR, FiVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, B7-H3, and a ligand that specifically binds with CD83, and the like. The term “costimulatory molecule” refers to the cognate binding partner on a T-cell that specifically binds with a costimulatory ligand, thereby mediating a costimulatory response by the T-cell, such as, but not limited to, proliferation. Costimulatory molecules are cell surface molecules other than antigen receptors or their ligands that are required for an efficient immune response. Costimulatory molecules include, but are not limited to an MHC class 1 molecule, BTLA and a Toll ligand receptor, as well as OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an extracellular, transmembrane of intracellular domain that is derived from a molecule, the derived domain retains sufficient structure such that is has the required function, e.g., the ability to generate a signal under the appropriate conditions. It does not connotate or include a limitation to a particular process of producing the extracellular domain, e.g., it does not mean that, to provide the extracellular domain, one must start with a subunit sequence and delete unwanted sequence, or impose mutations, to arrive at the extracellular domain.

By “dimerization domain” is meant a domain that binds a cognate dimerization domain either constitutively or inducibly. Such cognate dimerization domains may be the same or similar to the initial dimerization domain (“homodimerization domains”) or may be heterologous to the initial dimerization domain (“heterodimerization domains”). In cases where the domains constitutively dimerize, such dimerization will typically occur provided that both domains are expressed in the same cellular compartment. In cases where the domains inducibly dimerize, such dimerization may require the presence of a “dimerization molecule.” A dimerization domain (e.g., a leucine zipper) can be as described in WO2007115230, WO2017027392, US20160257721, WO2016055551, all of which are incorporated by reference herein.

“Dimerization molecule,” as that term is used herein, refers to a molecule that promotes the association of a first dimerization domain with a second dimerization domain. In embodiments, the dimerization molecule does not naturally occur in the subject, or does not occur in concentrations that would result in significant dimerization. In some embodiments, the dimerization molecule is a small molecule, e.g., rapamycm or a rapaiogue.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources. Further, as used herein, the terms “antibody” and “antibody molecule” refer to an immunoglobulin chain or fragment thereof, comprising at least one immunoglobulin variable domain sequence. The term “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies such as sdAb (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are linked via a short flexible polypeptide linker, and capable of being expressed as a single polypeptide chain, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

“Heavy chain variable region” or “V_{H}” with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The portion of the proteins of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al, 1988, Science 242:423-426), In one aspect, the antigen binding domain of a composition of the disclosure comprises an antibody fragment. In a further aspect, the protein comprises an antibody fragment that comprises a scFv.

The antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al, 1988, Science 242:423-426). In one aspect, the antigen binding domain of the disclosure comprises an antibody fragment. In a further aspect, the protein comprises an antibody fragment that comprises a scFv. The precise amino acid sequence boundaries of a given CDR can be determined using any of a number of well-known schemes, including those described by Kabat et ai. (1991), “Sequences of Proteins of Immunological Interest,” 5th Ed. Public Health Sen′ ice, National Institutes of Health, Bethesda, Md. (“Kabat” numbering scheme), Al-Lazikani et al., (1997) 1 MB 273,927-948 (“Chothia” numbering scheme), or a combination thereof.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (“K”) and lambda (“L”) light chains refer to the two major antibody light chain isotypes.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The term “4-1BB” refers to a member of the TNFR superfamily with an amino acid sequence provided as GenBank Acc. No. AA A62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a on-human species, e.g., mouse, rodent, monkey, ape and the like. In one aspect, the “4-1BB costimulatory domain” is the sequence provided as herein or the equivalent residues from a non-human species, e.g., mouse, rodent, monkey, ape and the like.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” as used herein refers to the return of a disease (e.g., cancer) or the signs and symptoms of a disease such as cancer after a period of improvement, e.g., after prior treatment of a therapy, e.g., cancer therapy.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

As used herein the specification, “subject” or “subjects” or “individuals” may include, but are not limited to, mammals such as humans or non-human mammals, e.g., domesticated, agricultural or wild, animals, as well as birds, and aquatic animals. In some cases, the term “subject” is intended to include living organisms in which an immune response can be elicited (e.g., mammals, human). “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

The portion of the activatable cell surface receptor polypeptide composition of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of an activatable cell surface receptor polypeptide composition of the disclosure comprises an antibody fragment. In a further aspect, the activatable cell surface receptor polypeptide or polypeptide complex comprises an antibody fragment that comprises an activatable scFv or an activatable sdAb.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “stimulatory molecule” or “stimulatory domain” refers to a molecule or portion thereof expressed by a T-cell that provides the primary cytoplasmic signaling sequence(s) that regulate primary activation of the TCR complex in a stimulatory way for at least some aspect of the T-cell signaling pathway. In one aspect, the primary signal is initiated by, for instance, binding of a TCR/CD3 complex with an MHC molecule loaded with peptide, and which leads to mediation of a T-cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the disclosure includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

The term “Chimeric Antigen Receptor” or alternatively a “CAR” refers to a recombinant polypeptide comprising an extracellular antigen binding domain in the form of a scFv, a transmembrane domain, and cytoplasmic signaling domains (also referred to herein as “an intracellular signaling domains”) comprising a functional signaling domain derived from a stimulatory molecule as described below. Generally, the intracellular signaling domain of a CAR is derived from the CD3 zeta chain that is normally found associated with the TCR complex. The CD3 zeta signaling domain can be fused with one or more functional signaling domains derived from at least one co-stimulatory molecule such as 4-1BB (i.e., CD137), CD27 and/or CD28.

### DESCRIPTION

Provided herein are compositions of matter and methods of use for the treatment of a disease such as cancer, using activatable cell surface receptor polypeptides or polypeptide complexes. An “activatable cell surface receptor polypeptides” or “activatable cell surface receptor” or “activatable receptor” or “ACSR” of the present disclosure includes a recombinant polypeptide derived from the various polypeptides comprising a component of the T Cell Receptor (TCR) and/or other T cell signaling molecule, that is generally capable of i) binding to a surface antigen on target cells when activated (and binding at a reduced level when not activated) and ii) inducing T cell signaling upon binding to the target antigen. As described herein, an ACSR provides substantial benefits as compared to existing Chimeric Antigen Receptors or T Cell Receptor (TCR) polypeptides.

Activation of activatable cell surface receptor polypeptides.

In some embodiments, the immune cells of the present disclosure contain on their cell surface a population of activatable cell surface receptor polypeptides, which, when activated, direct the immune cell to interact with a cell, such as a tumor cell, containing on its surface a target antigen, which is bound by an extracellar antigen-recognition domain of the activatable cell surface receptor polypeptide. Typically, an antigen-recognition domain is inactive outside of the tissue containing cells expressing the surface target antigen, e.g., typically a tumor environment containing a population of tumor cells having the target antigen on the cell surface.

Architecture of antigen-recognition domains.

Binding Pairs.

In a first embodiment, an extracellular antigen-recognition domain contains at least two binding pairs, a first binding pair and a second binding pair. Typically, each binding pair has an active domain and an inactive domain, and upon activation, the active domain of each binding pair becomes associated with the other, while the two inactive domains are physically separated from the associated active domains.

Linker Domains.

In some embodiments, the active domain and the inactive domain of each binding pair are separated by a linker domain.

Methods of Activation

Protease Cleavage Sites.

Typically, one or more domains of the extracellular antigen-recognition domain contains one or more protease cleavage sites.

In some embodiments, the first binding pair comprises a first V_{H} domain, a first linker domain comprising a first protease cleavage site, and an inactive first V_{L} domain, wherein the first linker domain is covalently linked to the first V_{H} domain and the inactive first V_{L} domain; and the second binding pair comprising a second V_{L} domain, a second linker domain comprising a second protease cleavage site, and an inactive second V_{H} domain wherein the second linker domain is covalently linked to the second V_{L} domain and the inactive second V_{H} domain, wherein the second V_{L} domain and the inactive second V_{H} domain are non-covalently associated, wherein the inactive first V_{H} domain and the inactive second V_{L} domain may be covalently associated by a third linker domain comprising a third protease cleavage site; wherein the first binding pair is covalently linked to a T Cell Receptor subunit alpha polypeptide, and wherein the second binding pair is linked to a TCR subunit beta polypeptide.

A polypeptide of the present disclosure may comprise two or more protease cleavage sites. In some cases, multiple protease cleavage sites can have the same sequence. In other cases, multiple protease cleavage sites can have different sequences. In some embodiments, two or more protease cleavage sites are different cleavage sites (e.g., capable of being recognized by different proteases). In some embodiments, two or more protease cleavage sites are the same cleavage site (e.g., capable of being recognized by the same protease).

Inactive Domains

In one aspect, the disclosure provides an activatable cell surface receptor comprising at least one or more inactive domains. In some embodiments, the inactive domains comprise protease cleavage sites. The inactive domains are alternatively referred to as inert domains. Examples of inactive domains include but are not limited to a variable heavy domain (VH), a variable light domain (VL), an scFv comprising a VH and a VL domain, a single domain antibody, or a variable domain of camelid derived nanobody (VHH), a VL single domain antibody, a non-Ig binding domain, i.e., antibody mimetic, such as anticalins, affilins, affibody molecules, affimers, affitins, alphabodies, avimers, DARPins, fynomers, kunitz domain peptides, and monobodies, a ligand or peptide. In some embodiments, an inactive domain is an inactive variable heavy (iVH) or an inactive variable light (iVL) domain. The activatable receptor is in a binding inactive configuration when the inactive domain is associated with at least one other domain of the protein such that said binding domain is prevented from binding its target or had reduced binding to its target.

In some embodiments, the one or more inactive domains each comprise at least one protease cleavage site. The protease cleavage sites are a stretch of amino acid sequences that are recognized and cleaved by any known protease, such as matrix metalloprotease (MMP9) or furin. In some cases, an inactive domain comprising a protease cleavage site recognized by MMP9 comprises the amino acid sequence PR(S/T)(L/I)(S/T) (SEQ ID NO: 27). In some cases, an inactive domain comprising a protease cleavage site recognized by MMP9 comprises the amino acid sequence LEATA (SEQ ID NO: 28). In some cases, the protease cleavage site is recognized in a sequence-specific manner by a MMP11. In some cases, the protease cleavage site recognized by a MMP11 comprises a polypeptide having an amino acid sequence GGAANLVRGG (SEQ IN NO: 29). In some cases, the protease cleavage site is recognized by a protease disclosed in Table 1. In some cases, the protease cleavage site recognized by a protease disclosed in Table 1 comprises a polypeptide having an amino acid sequence selected from a sequence disclosed in Table 1 (SEQ ID NOS: 25-66).

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease (e.g., trypsin), elastase-like protease, subtilisin-like protease, actinidain, bromelain, calpain, caspases, caspase-3, Mirl-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26).

Proteases are known to be secreted by some diseased cells and tissues, for example tumor or cancer cells, creating a microenvironment that is rich in proteases or a protease-rich microenvironment. Alternatively or in addition, proteases can be expressed on the surface of cells, for example cells surrounding a tumor or cancer cells, creating a microenvironment that is rich in proteases. In some cases, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen-binding proteins to have additional specificity because T cells will not be bound by the antigen binding protein except in the protease rich microenvironment of the targeted cells or tissue.

Activatable Cell Surface Receptors

In some embodiments, the activatable cell surface receptors described herein comprise a first and a second polypeptide chain, each polypeptide comprising an inactive variable domain, a variable heavy domain (VH) or a variable light domain (VL), and one or more additional domains (e.g., a transmembrane domain, an intracellular signaling domain, etc.). In some embodiments, the inactive domain is a variable light domain (iVL) or a variable heavy domain (iVH). In some embodiments, the inactive domains iVL and iVH each comprise at least one protease cleavage site which is cleaved by a protease to result in activation of the activatable cell surface receptors described herein.

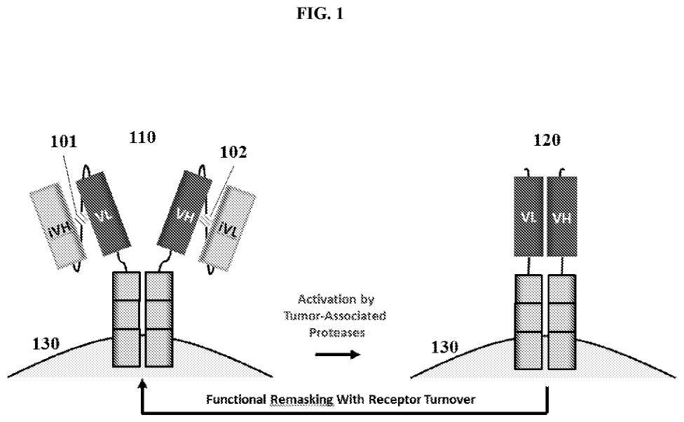

In some embodiments, the receptor comprises iVL, a VH, an iVH, and a VL, which VH and VL may associate to form the target-binding domain of the activatable receptor, as shown in FIG. 1. FIG. 1 shows a schematic of an example activatable cell surface receptor of the present disclosure. Inactive receptor 110 comprises a VL connected to an iVH via a linker comprising a protease cleavage site 101. Receptor 110 also comprises a VH connected to an iVL via a linker comprising a protease cleavage site 102. Receptor 110 also comprises a transmembrane domain and an intracellular signaling domain. Receptor 110 is provided to a T-cell 130. Upon exposure to a tumor environment, the protease cleavage sites 101 and 102 are cleaved by tumor-associated proteases, thereby activating the receptor, generating active receptor 120. In some embodiments, protease cleavage sites 101 and 102 are cleaved by different proteases. In some embodiments, protease cleavage sites 101 and 102 are cleaved by the same protease. Active receptor 120 now comprises a VH and VL, which are associated and can function as an active antigen-binding domain. As receptor 120 is internalized by T-cell 130, new receptors are generated which are inactive, thereby generating an inactive receptor 110.

In some embodiments, the activatable cell surface receptor disclosed herein comprises a single chain variable fragment (scFv). In some embodiments, an scFv comprises an inactive domain, and a variable light domain (VL) or variable heavy domain (VH). In some embodiments, an inactive domains is an iVL or iVH, and an iVL or iVH comprises at least one protease cleavage site which is cleaved by a protease to result in activation of the activatable cell surface receptors described in this embodiment. The protease cleavage sites of the inactive domains are, in some cases, within the complementarity determining regions of those domains.

In some embodiments, an scFv comprises an iVH domain and a VL domain. In some embodiments, an scFv comprises an iVL domain and a VH domain. An activatable receptor can comprise two scFv regions. In some embodiments, a first scFv comprises an iVL domain and a VH domain; and a second scFv comprises an iVH domain and a VL domain, wherein upon cleavage of at least one protease cleavage site in iVH and iVL the VL and VH domains associate to form an active monovalent target-binding protein.

The inactive variable domains iVL and iVH each comprises complementarity determining regions (CDRs) CDRL1, CDRL2, CDRL3, and CDRH1, CDRH2, CDRH3, respectively, and the at least one protease cleavage site is, in certain embodiments, located within said complementarity determining regions. It is contemplated that, in some embodiments, the CDRs of iVL and/or iVH further comprise one or more mutations that prohibit the binding of said domains to a target. In some embodiments, iVL and/or iVH do not comprise any mutations and naturally have no affinity for a given target.

The receptors described herein are binding-inactive in the two-armed form, and only bind target protein when in the monovalent form. In some embodiments, the receptors described herein do not have target-domain binding capability until at least one protease cleavage site in iVL and at least one cleavage site in iVH are cleaved by a protease and the VH and VL domains associate with each other to form an active receptor. In some embodiments, the proteins do not have target-domain binding capability until all the protease cleavage sites in iVH and iVL are cleaved and the VH and VL domains associate with each other to form an active receptor.

In some embodiments, the VH and iVL domains are connected via an internal linker. In some embodiments the VL and iVH domains are connected via an internal linker. In embodiments where the receptor comprises an iVL, a VH, an iVH, and VL, the linkers are as follows: L1 links iVL and VH, and L2 links VL and iVH. In some embodiments, linkers (e.g., L1, L2) each independently comprises at least one protease cleavage site which is cleaved by a protease to result in activation of the activatable cell surface receptors described herein

In some embodiments, linkers have an optimized length and/or amino acid composition. In some embodiments, linkers are 3-200 amino acids in length. In some embodiments, linkers have the same length or amino acid composition. In other embodiments, linkers have different amino acid compositions. In other embodiments, linkers have different lengths. In certain embodiments, internal linkers are “short”, i.e., consist of 0, 1, 2, 3, 4, 5, 6, 7, 8, 9, 10, 11 or 12 amino acid residues. Thus, in certain instances, the linkers consist of about 12 or less amino acid residues. In the case of 0 amino acid residues, the linker is a peptide bond. In certain embodiments, linkers consist of 15, 20 or 25 amino acid residues. In some embodiments, the linkers consist of about 3 to about 15, for example 8, 9 or 10 contiguous amino acid residues. Regarding the amino acid composition of the linkers, peptides are selected with properties that confer flexibility to the antigen-binding proteins, do not interfere with the target-binding domain as well as resist cleavage from proteases, unless the protease cleavage sites are located within the linkers. Examples of internal linkers suitable for linking the domains in the antigen-binding proteins include but are not limited to (GS)ₙ, (GGS)ₙ, (GGGS)ₙ, (GGSG)ₙ, (GGSGG)ₙ, or (GGGGS)ₙ, wherein n is 1, 2, 3, 4, 5, 6, 7, 8, 9, or 10. In one embodiment, a linker is (GGGGS)₄ or (GGGGS)₃.

In certain instances, one or more of the linkers comprise protease cleavage sites. Such protease sensitive linkers are, in certain embodiments, sensitive to protease present in specific tissue or intracellular compartments (MMPs, furin, cathepsin B). Example sequences for such protease sensitive cleavable linkers include but are not limited to (PLGLWA)ₙ, (RVLAEA)ₙ; (EDVVCCSMSY)ₙ, (GGIEGRGS)ₙ, which are recognized by MMP-1, and (GFLG)ₙ, which is recognized by furin. The linkers containing protease cleavage sites play a role in activation of the activatable cell surface receptor. In some embodiments, the binding protein is activated upon cleavage of the protease sites in iVL, iVH, and/or one or more of linkers. In some embodiments, an activatable receptor is not activated until at least one cleavage site in at least one of the linkers is cleaved.

In some embodiments, a VH, and/or an iVL may comprise one or more mutations for improving the affinity of a VH for an iVL. In some embodiments, a VL, and/or an iVH may comprise one or more mutations for improving the affinity of a VL for an iVH. In some embodiments, mutations are formed which enable the formation of a salt bridge between a VH and iVL and/or between a VL and iVH. In one example, a Q residue is mutated to an E residue on a VH, while a Q residue is mutated to a K residue on an iVL, thereby generating a salt bridge between the E and K residues. In another example, a Q residue is mutated to an E residue on a VL, while a Q residue is mutated to a K residue on an iVH, thereby generating a salt bridge between the E and K residues.

Target Antigens

The extracellular antigen-recognition domain typically binds to a target antigen. A target antigen is involved in and/or associated with a disease, disorder or condition. In particular, a target antigen associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell. The design of the activatable proteins described herein allows the extracellular binding domain to a target antigen to be flexible in that the binding domain to a target antigen can be any type of binding domain, including but not limited to, domains from a monoclonal antibody, a polyclonal antibody, a recombinant antibody, a human antibody, a humanized antibody. In some embodiments, the binding domain to a target antigen is a single chain variable fragments (scFv), single-domain antibody such as a heavy chain variable domain (VH), a light chain variable domain (VL), a variable domain (VHH) of camelid derived single domain antibody, and a VL single domain antibody. In other embodiments, the binding domain to a target antigen is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to a target antigen is a ligand or peptide that binds to or associates with a target antigen. In yet further embodiments, the binding domain to a target antigen is a knottin. In yet further embodiments, the binding domain to a target antigen is a small molecular entity.

Cancer Associated Antigens

The present disclosure further provides immune effector cells (e.g., T cells, NK cells) that are engineered to contain one or more activatable cell surface receptor polypeptides that direct the immune effector cells to a cancer cell. This is achieved through an extracellular antigen binding domain on the protein that is specific for a cancer associated antigen, and is selectively activated in a cancer cell environment. There are two classes of cancer associated antigens (tumor antigens) that can be targeted by the proteins of the instant disclosure: (1) cancer associated antigens that are expressed on the surface of cancer cells; and (2) cancer associated antigens that itself is intracelluar, however, a fragment of such antigen (peptide) is presented on the surface of the cancer cells by MHC. Accordingly, the present disclosure, in certain embodiments, provides activatable cell surface receptor polypeptides that target the following exemplary cancer associated antigens (tumor antigens): CD 19, CD 123, CD22, CD30, CD 171, CS-1, CLL-1 (CLECL1), CD33, CD166, CD171, EGFRvDI, GD2, GD3, BCMA, Tn Ag, PSMA, ROR1, FLT3, FAP, TAG72, CD38, CD44v6, CEA, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC 1, EGFR, NCAM, Prosiase, PAP, ELF2M, Ephrin B2, 1GF-1 receptor, CA1X, LMP2, g 100, bcr-abl, tyrosinase, EphA2, Fucosyl GM1, sLe, GM3, TGS5, UMWMAA, o-acetyl-GD2, Folate receptor beta, TEM1/CD248, TEM7R, CLD 6, TSHR, GPRC5D, CXORF61, CD97, CD 179a, ALK, Poiysialic acid, PLAC 1, GloboH, NY-BR-1, UPK2, HAVCR1. ADRB3, PANX3, GPR20, LY6K, OR51 E2, TARP, WT1, NY-ESO-1, LAGE-1a, legumain, HPV E6, E7, MAGE-A1, ETV6-AML, sperm protein 17, XAGE1, Tie 2, MAD-CT-1, MAD-CT-2, Fos-related antigen 1, p53, p53 mutant, prostein, survivin and telornerase, PCTA-1/Galectin 8, MelanA/MART1, Ras mutant, hTERT, sarcoma translocation breakpoints, ML-IAP, ERG (TMPRSS2 ETS fusion gene), NA 17, PAX3, Androgen receptor, Cyclin B 1, MYCN, RhoC, TRP-2, CYP 1 B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC 12A, BST2, EMR2, LY75, GPC3, FCRL5, AXL, IGF-1R, CD25, CD49C, gpA33, MUC-16, 1-92-LFA-3, CD52, DL44, HVEM, LIF-R, STEAP1, Alpha-4, CD56, DLK1, Hyaluronidase, Lewis X, STEAP2, integrin, Alpha-V, CD64, DLL4, ICOS, LIGHT, TAG-72, integrin, alpha4beta1, CD70, DPP-4, IFNalpha, LRP4, TAPA1, integrin, alpha4beta7, CD71, DSG1, IFNbeta, LRRC26, TGFbeta, integrin, AGR2, CD74, EGFR, IFNgamma, MCSP, TIGIT, Anti-Lewis-Y, EGFRviii, IgE, TIM-3, Apelin J receptor, CD80, Endothelin B receptor (ETBR), IgE Receptor (FceRI), MRP4, TLR2, APRIL, CD81, ENPP3, IGF, MUC1, TLR4, B7-H4, CD86, EpCAM, IGF1R, Mucin-16 (MUC16, CA-125), TLR6, BAFF, CD95, EPHA2, IL1B, Na/K ATPase, TLR7, BTLA, CD117, EPHB2, IL1R, Neutrophil elastase, TLR8, C5 complement, CD125, ERBB3, IL2, NGF, TLR9, C-242, CD132 (IL-2RG), F protein of RSV, IL11, Nicastrin, TMEM31, CA9, CD133, FAP, IL12, Notch receptors, TNFalpha, CA19-9 (Lewis a), CD137, FGF-2, IL12p40, Notch 1, TNFR, CD138, Carbonic anhydrase 9, FGF8, IL-12R, IL-12Rbeta1, Notch 2, TNFRS12A, CD2, CD166, FGFR1, IL13, Notch 3, TRAIL-R1, CD3, CD172A, FGFR2, IL13R, Notch 4, TRAIL-R2, CD6, CD248, FGFR3, IL15, NOV, Transferrin, CD9, CDH6, FGFR4, IL17, OSM-R, Transferrin receptor, CD11a, CEACAM5 (CEA), Folate receptor, IL18, OX-40, TRK-A, CD19, CEACAM6 (NCA-90), GAL3ST1, IL21, PAR2, TRK-B, CD20, CLAUDIN-3, G-CSF, IL23, PDGF-AA, uPAR, CD22, CLAUDIN-4, CLAUDIN-18, G-CSFR, IL23R, PDGF-BB, VAP1, CD24, cMet, GD2, IL27/IL27R (wsxl), PDGFRalpha, VCAM-1, CD25, Collagen, GITR, IL29, PDGFRbeta, VEGF, CD27, Cripto, GLUT1, IL-31R, PD-1, VEGF-A, CD28, CSFR, GLUT4, IL31/IL31R, PD-L1, VEGF-B, CD30, CSFR-1, GM-CSF, IL2R, PD-L2, VEGF-C, CD33, CTLA-4, GM-CSFR, IL4, Phosphatidylserine, VEGF-D, CD38, CTGF, GPIIb/IIIa receptors, IL4R, P1GF, VEGFR1, CD40, CXCL10, Gp130, IL6, IL6R, PSCA, VEGFR2, CD40L, CXCL13, GPIIB/IIIA, Insulin receptor, VEGFR3, CD41, CXCR1, GPNMB, Jagged ligands, RAAG12, VISTA, CD44, CXCR2, GRP78, Jagged 1, RAGE, WISP-1, CD44v6, Jagged 2, SLC44A4, WISP-2, CD47, CXCR4, HGF, LAG-3, Sphingosine 1 phosphate, WISP-3, CD51, CYR61, hGH, Liv-1 (SLC39A6), 5T4, and IGLL1.

Tumor-Supporting Antigens

In some embodiments, a chimeric protein (e.g., an activatable receptor) as described herein comprises an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. In embodiments, the stromal cell antigen is chosen from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD 11b, CD14, CD 15, and CD66b. Accordingly, in some embodiments, the tumor-supporting antigen is chosen from one or more of: bone marrow stromal cell antigen 2 (BST2), fibroblast activation protem (FAP) or tenascin, CD33, CD 11b, CD14, CD 15, and CD66b.

T Cell Binding Domains

The specificity of the response of T cells is mediated by the recognition of antigen (displayed in context of a major histocompatibility complex, WIC) by the TCR. As part of the TCR, CD3 is a protein complex that includes a CD3 gamma chain, a CD3 delta chain, and two CD3epsilon chains which are present on the cell surface. CD3 associates with the alpha and the beta chains of the TCR as well as CD3 zeta altogether to comprise the complete TCR. Clustering of CD3 on T cells, such as by immobilized anti-CD3 antibodies leads to T cell activation similar to the engagement of the T cell receptor but independent of its clone-typical specificity.

In one aspect, the activatable cell surface receptor polypeptides described herein comprise a domain which specifically binds to CD3. In one aspect, the activatable cell surface receptor polypeptides described herein comprise a domain which specifically binds to human CD3. In some embodiments, the activatable cell surface receptor polypeptides described herein comprise a domain which specifically binds to CD3 gamma. In some embodiments, the activatable cell surface receptor polypeptides described herein comprise a domain which specifically binds to CD3 delta. In some embodiments, the activatable cell surface receptor polypeptides described herein comprise a domain which specifically binds to CD3 epsilon.

In one aspect, the activatable cell surface receptor polypeptides described herein comprise a domain which specifically binds to a T cell. In further embodiments, the activatable cell surface receptor polypeptides described herein comprise a domain which specifically binds to the TCR. In certain instances, the activatable cell surface receptor polypeptides described herein comprise a domain which specifically binds the alpha chain of the TCR. In certain instances, the activatable cell surface receptor polypeptides described herein comprise a domain which specifically binds the beta chain of the TCR. In further embodiments, the activatable cell surface receptor polypeptides described herein comprise a domain which specifically binds to CD4. In further embodiments, the activatable cell surface receptor polypeptides described herein comprise a domain which specifically binds to CD8.

Humanized Activatable Cell Surface Receptor Polypeptide

In some aspects, an activatable cell surface receptor polypeptide as described herein comprises a humanized antibody or antibody fragment, where specific sequences or regions of the antibody are modified to increase similarity to an antibody naturally produced in a human or fragment thereof. In one aspect, the antigen binding domain is humanized.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise a natural or synthetic ligand specifically recognizing and binding the target antigen can be used as antigen binding domain for the activatable cell surface receptor.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a V_{H}4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a composition of the disclosure that comprises an antibody fragment is humanized with retention of high affinity for the target antigen and other favorable biological properties. According to one aspect of the disclosure, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind human target antigen. In some embodiments, a humanized antibody or antibody fragment may have improved affinity and/or specificity of binding to human target antigen.

In one aspect, the human target antigen binding domain is characterized by particular functional features or properties of an antibody or antibody fragment.

Also provided herein are methods for obtaining an antibody antigen binding domain specific for a target antigen (e.g., target antigen described elsewhere herein for targets of fusion moiety binding domains), the method comprising providing by way of addition, deletion, substitution or insertion of one or more amino acids in the amino acid sequence of a V_{H} domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). scFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding is also required to bring the two variable regions together to form a functional epitope binding site. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

A scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

Stability and Mutations

The stability of an activatable cell surface receptor polypeptide, such as an scFv molecule (e.g., soluble scFv) can be evaluated in reference to the biophysical properties (e.g., thermal stability) of a conventional control scFv molecule or a full length antibody. In one embodiment, the activatable cell surface receptor polypeptide comprises a humanized or human scFv. In some embodiments, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described in more detail below.

Mutations in the scFv (arising through humanization or direct mutagenesis of the soluble scFv) alter the stability of the scFv and improve the overall stability of the scFv. Stability of the humanized scFv is compared against the murine scFv using measurements such as T_{M}, temperature denaturation and temperature aggregation. In one embodiment, binding domain, e.g., a scFv, comprises at least one mutation arising from the humanization process such that the mutated scFv confers improved stability. In another embodiment, the binding domain, e.g., scFv comprises at least 1, 2, 3, 4, 5, 6, 7, 8, 9, 10 mutations arising from the humanization process such that the mutated scFv confers improved stability to the construct.

In various aspects, the antigen binding domain of the polypeptide is engineered by modifying one or more amino acids within one or both variable regions (e.g., V_{H} and/or V_{L}), for example within one or more CDR regions and/or within one or more framework regions. In one specific aspect, the composition of the disclosure comprises an antibody fragment. In a further aspect, that antibody fragment comprises a scFv.

It will be understood by one of ordinary skill in the art that the antibody or antibody fragment of the disclosure may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In one aspect, the present disclosure contemplates modifications of the starting antibody or fragment (e.g., scFv) amino acid sequence that generate functionally equivalent molecules. For example, the V_{H} or V_{L} e.g., scFv, comprised in the composition can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the binding domain, e.g., scFv. The present disclosure contemplates modifications of the entire construct, e.g., modifications in one or more amino acid sequences of the various domains of the construct in order to generate functionally equivalent molecules. The construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting construct.

Extracellular Domain

In various embodiments, the extracellular domain of the activatable cell surface receptor polypeptide described herein comprises an extracellular antigen recognition polypeptide. The extracellular antigen binding domain can be made up of binding domain pairs, wherein each binding domain of the pair comprises various combinations of a V_{H} domain, V_{L} domain, a stabilization domain, and a linker. In some embodiments, the extracellular antigen-recognition polypeptide is covalently connected to a T Cell Receptor subunit binding domain or a CD4 binding domain or a CD8 binding domain, wherein binding of the T Cell Receptor subunit binding domain or a CD4 binding domain or a CD8 binding domain does not substantially activate a T Cell Receptor present on the engineered immune cell.

In additional embodiments, the extracellular antigen recognition polypeptide comprises an extracellular antigen-recognition polypeptide that specifically binds to a peptide or a polypeptide present in a tumor antigen, wherein the extracellular antigen-recognition polypeptide comprises an antigen binding domain, a stabilization domain, and a linker domain, wherein the stabilization domain and/or the linker domain comprise a protease cleavage site.

In yet other embodiments, the extracellular antigen recognition polypeptide comprises (i) a V_{HH} domain, (ii) a linker domain, (iii) an inhibitory domain or a masking domain.

In some embodiments, the extracellular antigen recognition polypeptide comprises (i) a VHH domain, (ii) a linker domain, (iii) an inhibitory domain or a masking domain, wherein the linker domain is covalently linked to the VHH domain and the inhibitory domain, and wherein the linker domain and/or the inhibitory domain include a protease cleavage site, and (iv) a T Cell Receptor subunit or portion thereof selected from the group consisting of TCR-alpha, TCR-beta, CD3-gamma, CD3-epsilon, and CD3-delta, or CD4 or CD8.

In certain embodiments, the linker domains comprise one or more protease cleavage sites. In certain embodiments, the stabilization domains comprise one or more protease cleavage sites. In certain embodiments, the inhibitory or masking domains comprise one or more protease cleavage sites.

In certain embodiments, the linker domains comprise amino acid sequences that are substrate for at least one tumor specific protease, such as a MMP. In some embodiments, the linker domain of each binding pair independently comprises an amino acid sequence selected from the group consisting of (GS)n, (GGS)n, (GSGGS)n, and (GGGS)n, where n is an integer of at least one.

In some embodiments, the linker domain of each binding pair independently comprises an amino acid sequence selected from the group consisting of GGSG (SEQ ID NO: 68), GGSGG (SEQ ID NO: 69), GSGSG (SEQ ID NO: 70), GSGGG (SEQ ID NO: 71), GGGSG (SEQ ID NO: 72), and GSSSG (SEQ ID NO: 73).

In some embodiments, the linker domain of each binding pair independently comprises the amino acid sequence GSSGGSGGSGGSG (SEQ ID NO: 74), GSSGGSGGSGG (SEQ ID NO: 75), GSSGGSGGSGGS (SEQ ID NO: 76), GSSGGSGGSGGSGGGS (SEQ ID NO: 77), GSSGGSGGSG (SEQ ID NO: 78), or GSSGGSGGSGS (SEQ ID NO: 79). In some embodiments, the linker domain of each binding pair independently comprises the amino acid sequence GSS (SEQ ID NO: 80), GGS (SEQ ID NO: 81), GGGS (SEQ ID NO: 82), GSSGT (SEQ ID NO: 83) or GSSG (SEQ ID NO: 84).

The inhibitory domain or the masking domain inhibits the binding of the antibody or antigen binding domain thereof to the tumor antigen when associated with the antibody or antigen binding domain thereof. In some embodiments, the inhibitory domain or the masking domain comprises further protease cleavage site or amino acid sequences that are substrates for a tumor specific protease, such as MMP.

In yet other embodiments, the extracellular domain of the activatable cell surface receptor polypeptide comprises a cleavable domain that is connected to the inhibitory or masking domain. In some embodiments, the cleavable domain and the inhibitory or masking domain are connected by a linker domain, which has amino acid sequence as described above. The cleavable domains have amino acid sequences that are substrates for one or more proteases. In some embodiments, the cleavable domain is a substrate for MMP2, MMP9, MMP14, MMP1, MMP3, MMP13, MMP17, MMP11, and/or MMP19. In some embodiments, the cleavable domain is a substrate for MMP2. In some embodiments, the cleavable domain is a substrate for MMP9. In some embodiments, the cleavable domain is a substrate for MMP14. In some embodiments, the cleavable domain is a substrate for two or more MMPs. In some embodiments, the cleavable domain is a substrate for at least MMP9 and MMP14. In some embodiments, the cleavable domain is a substrate for at least MMP2 and MMP9. In some embodiments, the cleavable domain is a substrate for at least MMP2 and MMP14. In some embodiments, the cleavable domain is a substrate for three or more MMPs. In some embodiments, the cleavable domain is a substrate for at least MMP2, MMP9, and MMP14. In some embodiments, the cleavable domain comprises two or more substrates for the same MMP. In some embodiments, the cleavable domain comprises at least two or more MMP2 substrates. In some embodiments, the cleavable domain comprises at least two or more MMP9 substrates. In some embodiments, the cleavable domain comprises at least two or more MMP14 substrates.

In some embodiments, the cleavable domain is a substrate for a protease and includes at least the sequence ISSGLLSS (SEQ ID NO: 85); QNQALRMA (SEQ ID NO: 86); AQNLLGMV (SEQ ID NO: 87); STFPFGMF (SEQ ID NO: 88); PVGYTSSL (SEQ ID NO: 89); DWLYWPGI (SEQ ID NO: 90); MIAPVAYR (SEQ ID NO: 91); RPSPMWAY (SEQ ID NO: 92); WATPRPMR (SEQ ID NO: 93); FRLLDWQW (SEQ ID NO: 94); LKAAPRWA (SEQ ID NO: 95); GPSHLVLT (SEQ ID NO: 96); LPGGLSPW (SEQ ID NO: 97); MGLFSEAG (SEQ ID NO: 98); SPLPLRVP (SEQ ID NO: 99); RMHLRSLG (SEQ ID NO: 100); LAAPLGLL (SEQ ID NO: 101); AVGLLAPP (SEQ ID NO: 102); LLAPSHRA (SEQ ID NO: 103); PAGLWLDP (SEQ ID NO: 104); ISSGLSS (SEQ ID NO: 105); ISSGL (SEQ ID NO: 106); ISSGLLS (SEQ ID NO: 107); ISSGLL (SEQ ID NO: 108); and/or VHMPLGFLGP (SEQ ID NO: 109).

In addition to the above exemplary sequences, the linker, the inhibitory domain or masking domain, the stabilization domain, and/or the cleavable domains are contemplated to have amino acid sequences that are substrates for one or more proteases, such as ADAMS, ADAMTS (ADAMS, ADAMS, ADAM10, ADAM12, ADAM15, ADAM17/TACE, ADAMDEC1, ADAMTS1, ADAMTS4, ADAMTS5); Aspartate proteases (e.g., BACE, Renin, Cathepsin D, Cathepsin E); Caspases (e.g., Caspase 1, Caspase 2, Caspase 3, Caspase 4, Caspase 5, Caspase 6, Caspase 7, Caspase 8, Caspase 9, Caspase 10, Caspase 14); Cysteine cathepsins (e.g., Cathepsin B, Cathepsin C, Cathepsin K, Cathepsin L, Cathepsin S, Cathepsin V/L2, Cathepsin X/Z/P); Cysteine proteinases (e.g., Cruzipain, Legumain, Otubain-2); KLKs (KLK4, KLK5, KLK6, KLK7, KLK8, KLK10, KLK11, KLK13, KLK14); Serine proteases (e.g., activated protein C, Cathepsin A, Cathepsin G, Chymase); coagulation factor proteases (e.g., FVIIa, FIXa, FXa, FXIa, FXIIa); Elastase; Granzyme B; Guanidinobenzoatase; HtrA1; Human Neutrophil; Aspartic cathepsins, Metallo proteinases, Elastase; Lactoferrin; Marapsin; NS3/4A; PACE4; Plasmin; PSA; tPA; Thrombin; Tryptase; uPA; TypII Transmembrane; Serine Proteases (TTSPS) (e.g., DESC1, DPP-4, FAP, Hepsin, Matriptase-2, MT-SP1/Matriptase, TMPRSS2, TMPRSS3, TMPRSS4); Metallo proteinases (e.g., Neprilysin, PSMA, BMP-1); MMPs (e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, MMP27).

Inhibitory Domain

In some cases, the activatable receptors described herein comprise a domain that inhibits binding of an antigen-binding domain to a target antigen until activation by a protease. In some embodiments, an inhibitory domain comprises a protease cleavage site. A protease cleavage site may be cleaved, for example, in a disease-specific microenvironment or in the blood of a subject at the protease cleavage site allowing the target antigen binding domain to bind to a target antigen on a target cell. In some embodiments, the inhibitory domain binds to the target binding domain. In some embodiments, the inhibitory domain blocks binding of the target binding domain to the target antigen.

The design of the activatable receptors described herein allows the inhibitory domain to be flexible in that the inhibitory domain inhibiting binding to a target antigen can be any type of binding domain, including, but not limited to, domains from a monoclonal antibody, a polyclonal antibody, a recombinant antibody, a human antibody, or a humanized antibody. In some embodiments, the inhibitory domain is a single chain variable fragment (scFv), single-domain antibody such as a heavy chain variable domain (VH), a light chain variable domain (VL), a variable domain (VHH) of camelid derived nanobody, and a VL single domain antibody. In other embodiments, the inhibitory domain is a non-Ig binding domain, e.g., antibody mimetic, such as anticalins, affilins, affibody molecules, affimers, affitins, alphabodies, avimers, DARPins, fynomers, kunitz domain peptides, and monobodies.

Transmembrane Domain

In general, a sequence contains an extracellular domain and a transmembrane domain encoded by a single genomic sequence. In alternative embodiments, a construct can be designed to comprise a transmembrane domain that is heterologous to the extracellular domain of the construct. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 or up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 or up to 15 amino acids of the intracellular region). In one aspect, the transmembrane domain is one that is associated with one of the other domains of the construct is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another construct on the T-cell surface. In a different aspect the amino acid sequence of the transmembrane domain may be modified or substituted so as to minimize interactions with the binding domains of the native binding partner present in the same construct.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the construct has bound to a target. A transmembrane domain of particular use in this disclosure may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154.

In some instances, the transmembrane domain can be attached to the extracellular region of the polypeptide, e.g., the extracellular activatable antigen binding domain of the polypeptide, via a hinge, e.g., a hinge from a human protein. For example, in one embodiment, the hinge can be a human immunoglobulin (Ig) hinge, e.g., an IgG4 hinge, or a CD8a hinge.

Hinge Region

In some cases, the activatable receptors of the present disclosure comprise a hinge region (also referred to herein as a “spacer”), where the hinge region is interposed between the target antigen binding domain and the transmembrane domain. In some cases, the hinge region is an immunoglobulin heavy chain hinge region. In some cases, the hinge region is a hinge region polypeptide derived from a receptor (e.g., a CD8-derived hinge region).

In some embodiments, the hinge region has a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

Suitable spacers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary spacers include glycine polymers (G)n, glycine-serine polymers (including, for example, (GS)n, (GSGGS)n and (GGGS)n, where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary spacers comprise amino acid sequences including, but not limited to, GGSG, GGSGG, GSGSG, GSGGG, GGGSG, GSSSG, and the like.

In some embodiments, a hinge region is an immunoglobulin hinge region. As non-limiting examples, an immunoglobulin hinge region can include one of the following amino acid sequences: DKTHT; CPPC; CPEPKSCDTPPPCPR (see, e.g., Glaser et al. (2005) J. Biol. Chem. 280:41494); ELKTPLGDTTHT; KSCDKTHTCP; KCCVDCP; KYGPPCP; EPKSCDKTHTCPPCP (human IgG1 hinge); ERKCCVECPPCP (human IgG2 hinge); ELKTPLGDTTHTCPRCP (human IgG3 hinge); SPNMVPHAHHAQ (human IgG4 hinge); and the like.

In some embodiments, the hinge region comprises an amino acid sequence of a human IgG1, IgG2, IgG3, or IgG4 hinge region. The hinge region can include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP; see, e.g., Yan et al. (2012) J. Biol. Chem. 287:5891.

In some embodiments, the hinge region comprises an amino acid sequence derived from human CD8; e.g., the hinge region comprises the amino acid sequence: TTTPAPRPPTPAPTIASQPLSLRPEACRPAAGGAVHTRGLDFACD, or a variant thereof.

Linkers

Optionally, a short oligo- or polypeptide linker, between 2 and 10 amino acids in length may form the linkage between the transmembrane domain and the cytoplasmic region of the polypeptide. A glycine-serine doublet provides a particularly suitable linker. For example, in one aspect, the linker comprises the amino acid sequence of GGGGSGGGGS (SEQ ID NO:120). In some embodiments, the linker is encoded by a nucleotide sequence of GGTGGCGGAGGTTCTGGAGGTGGAGGTTCC (SEQ ID NO: 121). In some embodiments, the linker comprises the amino acid sequence of GG (SEQ ID NO: 122). In some embodiments, the linker comprises the amino acid sequence of GGSGGS (SEQ ID NO: 67).

Cytoplasmic Domain

The cytoplasmic domain of the polypeptide can include an intracellular signaling domain, as the intracellular signaling domain is generally responsible for activation of at least one of the normal effector functions of the immune cell in which the polypeptide has been introduced. The term “effector function” refers to a specialized function of a cell. Effector function of a T-cell, for example, may be cytolytic activity or helper activity including the secretion of cytokines. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the polypeptide of the disclosure include the cytoplasmic sequences of the T-cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of naive T-cells and that a secondary and/or costimulatory signal is required. Thus, naïve T-cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the disclosure include those of CD3 zeta, FcR gamma, FcR beta, CD3 gamma, CD3 delta, CD3 epsilon, CD5, CD22, CD79a, CD79b, and CD66d. In one embodiment, an activatable cell surface receptor polypeptide of the disclosure comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human activatable cell surface receptor polypeptide-T-cells in vitro and augments human T-cell persistence and antitumor activity in vivo (Song et al. Blood. 2012; 119(3):696-706).

The intracellular signaling sequences within the cytoplasmic portion of the polypeptide of the disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the engineered cell described herein can further comprise a second activatable cell surface receptor polypeptide, e.g., a second activatable cell surface receptor polypeptide that includes a different antigen binding domain, e.g., to the same target (Human target antigen) or a different target (e.g., CD123). In one embodiment, when the activatable cell surface receptor polypeptide-expressing cell comprises two or more different activatable cell surface receptor polypeptides, the antigen binding domains of the different activatable cell surface receptor polypeptides can be such that the antigen binding domains do not interact with one another. For example, a cell expressing a first and second activatable cell surface receptor polypeptide can have an antigen binding domain of the first activatable cell surface receptor polypeptide, e.g., as a fragment, e.g., a scFv, that does not form an association with the antigen binding domain of the second activatable cell surface receptor polypeptide, e.g., the antigen binding domain of the second activatable cell surface receptor polypeptide is a V_{HH}.

In another aspect, the activatable cell surface receptor polypeptide expressing cell described herein can further express another agent, e.g., an agent which enhances the activity of an activatable cell surface receptor polypeptide-expressing cell. For example, in one embodiment, the agent can be an agent which inhibits an inhibitory molecule. Inhibitory molecules, e.g., PD1, can, in some embodiments, decrease the ability of an activatable cell surface receptor polypeptide-expressing cell to mount an immune effector response. Examples of inhibitory molecules include PD1, PD-L1, CTLA4, TIM3, LAG3, VISTA, BTLA, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T-cells and myeloid cells (Agata et al. 1996 Int. Immunol 8:765-75). Two ligands for PD1, PD-L1 and PD-L2 have been shown to downregulate T-cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In another aspect, the present disclosure provides a population of activatable cell surface receptor polypeptide-expressing T-cells, e.g., activatable cell surface receptor polypeptide-T-cells. In some embodiments, the population of activatable cell surface receptor polypeptide-expressing T-cells comprises a mixture of cells expressing different activatable cell surface receptor polypeptides. for example, in one embodiment, the population of activatable cell surface receptor polypeptide-T-cells can include a first cell expressing an activatable cell surface receptor polypeptide having an human target antigen binding domain described herein, and a second cell expressing an activatable cell surface receptor polypeptide having a different human target antigen binding domain, e.g., an human target antigen binding domain described herein that differs from the anti-CD19 binding domain in the activatable cell surface receptor polypeptide expressed by the first cell. As another example, the population of activatable cell surface receptor polypeptide-expressing cells can include a first cell expressing an activatable cell surface receptor polypeptide that includes an human target antigen binding domain, e.g., as described herein, and a second cell expressing an activatable cell surface receptor polypeptide that includes an antigen binding domain to a target other than Human target antigen (e.g., another tumor-associated antigen).

In another aspect, the present disclosure provides a population of cells wherein at least one cell in the population expresses an activatable cell surface receptor polypeptide having a human target antigen domain described herein, and a second cell expressing another agent, e.g., an agent which enhances the activity of an activatable cell surface receptor polypeptide-expressing cell. For example, in one embodiment, the agent can be an agent which inhibits an inhibitory molecule. Inhibitory molecules, e.g., can, in some embodiments, decrease the ability of an activatable cell surface receptor polypeptide-expressing cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding activatable cell surface receptor polypeptides. The present disclosure also includes an activatable cell surface receptor polypeptide encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the activatable cell surface receptor polypeptide.

In one aspect the human target antigen activatable cell surface receptor polypeptide is encoded by a messenger RNA (mRNA). In one aspect the mRNA encoding the human target antigen activatable cell surface receptor polypeptide is introduced into a T-cell for production of an activatable cell surface receptor polypeptide-T-cell. In one embodiment, the in vitro transcribed RNAn activatable cell surface receptor polypeptide can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is an activatable cell surface receptor polypeptide of the present disclosure. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

In some cases, PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In a preferred embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning is highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

Nucleic Acid Constructs Encoding an Activatable Cell Surface Receptor Polypeptide

The present disclosure also provides nucleic acid molecules encoding one or more activatable cell surface receptor polypeptide constructs described herein. In one aspect, the nucleic acid molecule is provided as a messenger RNA transcript. In one aspect, the nucleic acid molecule is provided as a DNA construct.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired activatable cell surface receptor polypeptide of the disclosure is an adenoviral vector (A5/35). In another embodiment, the expression of nucleic acids encoding activatable cell surface receptor polypeptides can be accomplished using of transposons such as sleeping beauty, crisper, CAS9, and zinc finger nucleases. June et al. 2009 Nature Reviews Immunology 9.10: 704-716, is incorporated herein by reference.

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193, incorporated by reference herein in their entireties).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing an activatable cell surface receptor polypeptide transgene in a mammalian T-cell is the EFla promoter. The native EFla promoter drives expression of the alpha subunit of the elongation factor-1 complex, which is responsible for the enzymatic delivery of aminoacyl tRNAs to the ribosome. The EFla promoter has been extensively used in mammalian expression plasmids and has been shown to be effective in driving an activatable cell surface receptor polypeptide expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline-regulated promoter.

In order to assess the expression of an activatable cell surface receptor polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY). A contemplated method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362, incorporated by reference herein in their entireties).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristoyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristoyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are Lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

The present disclosure further provides a vector comprising an activatable cell surface receptor polypeptide encoding nucleic acid molecule. In one aspect, an activatable cell surface receptor polypeptide vector can be directly transduced into a cell, e.g., a T-cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the activatable cell surface receptor polypeptide construct in mammalian T-cells. In one aspect, the mammalian T-cell is a human T-cell.

Cells

In one embodiment, the present disclosure provides a cell comprising the activatable cell surface receptor of the present disclosure. The cell may be a mammalian cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, HuT-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte (T cell) obtained from an individual. As another example, the cell is a cytotoxic cell obtained from an individual. As another example, the cell is a stem cell or progenitor cell obtained from an individual. A further example comprises an engineered human T cell which is infused into a patient without being rejected by the patient's immune system.

Sources of T-Cells

Prior to expansion and genetic modification, a source of T-cells is obtained from a subject. T-cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present disclosure, any number of T-cell lines available in the art, may be used. In certain aspects of the present disclosure, T-cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T-cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the disclosure, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In one aspect, T-cells are isolated from peripheral blood lymphocytes by lysing the red blood cells and depleting the monocytes, for example, by centrifugation through a PERCOLL™ gradient or by counterflow centrifugal elutriation. A specific subpopulation of T-cells, such as CD3+, CD28+, CD4+, CD8+, CD45RA+, and CD45RO+ T-cells, can be further isolated by positive or negative selection techniques. For example, in one aspect, T-cells are isolated by incubation with anti-CD3/anti-CD28 (e.g., 3×28)-conjugated beads, such as DYNABEADS' M-450 CD3/CD28 T, for a time period sufficient for positive selection of the desired T-cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T-cells in any situation where there are few T-cells as compared to other cell types, such in isolating tumor infiltrating lymphocytes (TIL) from tumor tissue or from immunocompromised individuals. Further, use of longer incubation times can increase the efficiency of capture of CD8+ T-cells. Thus, by simply shortening or lengthening the time T-cells are allowed to bind to the CD3/CD28 beads and/or by increasing or decreasing the ratio of beads to T-cells (as described further herein), subpopulations of T-cells can be preferentially selected for or against at culture initiation or at other time points during the process. Additionally, by increasing or decreasing the ratio of anti-CD3 and/or anti-CD28 antibodies on the beads or other surface, subpopulations of T-cells can be preferentially selected for or against at culture initiation or at other desired time points. The skilled artisan would recognize that multiple rounds of selection can also be used in the context of this disclosure. In certain aspects, it may be desirable to perform the selection procedure and use the “unselected” cells in the activation and expansion process. “Unselected” cells can also be subjected to further rounds of selection.

Enrichment of a T-cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T-cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T-cell population can be selected that expresses one or more of IFN-γ, TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No. WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T-cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T-cells that normally have weaker CD28 expression.

In a related aspect, it may be desirable to use lower concentrations of cells. By significantly diluting the mixture of T-cells and surface (e.g., particles such as beads), interactions between the particles and cells is minimized. This selects for cells that express high amounts of desired antigens to be bound to the particles. For example, CD4+ T-cells express higher levels of CD28 and are more efficiently captured than CD8+ T-cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T-cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present disclosure.

Also contemplated in the context of the disclosure is the collection of blood samples or apheresis product from a subject at a time period prior to when the expanded cells as described herein might be needed. As such, the source of the cells to be expanded can be collected at any time point necessary, and desired cells, such as T-cells, isolated and frozen for later use in T-cell therapy for any number of diseases or conditions that would benefit from T-cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T-cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T-cells are obtained from a patient directly following treatment that leaves the subject with functional T-cells. In this regard, it has been observed that following certain cancer treatments, in particular treatments with drugs that damage the immune system, shortly after treatment during the period when patients would normally be recovering from the treatment, the quality of T-cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T-cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T-cells, B cells, dendritic cells, and other cells of the immune system.

Activation and Expansion of T Cells

T-cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T-cells of the disclosure may be expanded by contact with a surface having attached thereto an agent that stimulates a CD3/TCR complex associated signal and a ligand that stimulates a costimulatory molecule on the surface of the T-cells. In particular, T-cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T-cells, a ligand that binds the accessory molecule is used. For example, a population of T-cells can be contacted with an anti-CD3 antibody and an anti-CD28 antibody, under conditions appropriate for stimulating proliferation of the T-cells. To stimulate proliferation of either CD4+ T-cells or CD8+ T-cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

T-cells that have been exposed to varied stimulation times may exhibit different characteristics. For example, typical blood or apheresed peripheral blood mononuclear cell products have a helper T-cell population (TH, CD4+) that is greater than the cytotoxic or suppressor T-cell population (TC, CD8+). Ex vivo expansion of T-cells by stimulating CD3 and CD28 receptors produces a population of T-cells that prior to about days 8-9 consists predominately of TH cells, while after about days 8-9, the population of T-cells comprises an increasingly greater population of TC cells. Accordingly, depending on the purpose of treatment, infusing a subject with a T-cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T-cell product for specific purposes.

Once an activatable cell surface receptor polypeptide is constructed, various assays can be used to evaluate the activity of the molecule, such as but not limited to, the ability to expand T-cells following antigen stimulation, sustain T-cell expansion in the absence of re-stimulation, and anti-cancer activities in appropriate in vitro and animal models. Assays to evaluate the effects of a human target antigen activatable cell surface receptor polypeptide are described in further detail below.

Western blot analysis of activatable cell surface receptor polypeptide expression in primary T-cells can be used to detect the presence of monomers and dimers (see, e.g., Milone et al., Molecular Therapy 17(8): 1453-1464 (2009)). Very briefly, T-cells (1:1 mixture of CD4⁺ and CD8⁺ T-cells) expressing the activatable cell surface receptor polypeptides are expanded in vitro for more than 10 days followed by lysis and SDS-PAGE under reducing conditions. activatable cell surface receptor polypeptides are detected by Western blotting using an antibody to a TCR chain. The same T-cell subsets are used for SDS-PAGE analysis under non-reducing conditions to permit evaluation of covalent dimer formation.

In vitro expansion of activatable cell surface receptor polypeptide⁺ T-cells following antigen stimulation can be measured by flow cytometry. For example, a mixture of CD4⁺ and CD8⁺ T-cells are stimulated with anti-CD3/anti-CD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T-cell subsets by flow cytometry (see, e.g., Milone et al., Molecular Therapy 17(8): 1453-1464 (2009)). Alternatively, a mixture of CD4+ and CD8+ T-cells are stimulated with alphaCD3/alphaCD28 coated magnetic beads on day 0, and transduced with activatable cell surface receptor polypeptide on day 1 using a bicistronic lentiviral vector expressing activatable cell surface receptor polypeptide along with eGFP using a 2A ribosomal skipping sequence. Cultures are re-stimulated with either CD19+K562 cells (K562-CD19), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/mL. GFP+ T-cells are enumerated by flow cytometry using bead-based counting (see, e.g., Milone et al., Molecular Therapy 17(8): 1453-1464 (2009)).

Sustained activatable cell surface receptor polypeptide+ T-cell expansion in the absence of re-stimulation can also be measured (see, e.g., Milone et al., Molecular Therapy 17(8): 1453-1464 (2009)). Briefly, mean T-cell volume (ft) is measured on day 8 of culture using a Coulter Multisizer III particle counter following stimulation with alphaCD3/alphaCD28 coated magnetic beads on day 0, and transduction with the indicated activatable cell surface receptor polypeptide on day 1.

Animal models can also be used to measure an activatable cell surface receptor polypeptide activity. For example, xenograft model using human CD19-specific activatable cell surface receptor polypeptide+ T-cells to treat a primary human pre-B ALL in immunodeficient mice can be used (see, e.g., Milone et al., Molecular Therapy 17(8): 1453-1464 (2009)). Very briefly, after establishment of ALL, mice are randomized as to treatment groups. Different numbers of engineered T-cells are coinjected at a 1:1 ratio into NOD/SCID/γ−/− mice bearing B-ALL. The number of copies of each vector in spleen DNA from mice is evaluated at various times following T-cell injection. Animals are assessed for leukemia at weekly intervals. Peripheral blood CD19+B-ALL blast cell counts are measured in mice that are injected with alphaCD19-zetan activatable cell surface receptor polypeptide+ T-cells or mock-transduced T-cells. Survival curves for the groups are compared using the log-rank test. In addition, absolute peripheral blood CD4+ and CD8+ T-cell counts 4 weeks following T-cell injection in NOD/SCID/γ−/− mice can also be analyzed. Mice are injected with leukemic cells and 3 weeks later are injected with T-cells engineered to express activatable cell surface receptor polypeptides by a bicistronic lentiviral vector that encodes the activatable cell surface receptor polypeptide linked to eGFP. T-cells are normalized to 45-50% input GFP+ T-cells by mixing with mock-transduced cells prior to injection, and confirmed by flow cytometry. Animals are assessed for leukemia at 1-week intervals. Survival curves for the activatable cell surface receptor polypeptide+ T-cell groups are compared using the log-rank test.

Dose dependent activatable cell surface receptor polypeptide treatment response can be evaluated (see, e.g., Milone et al., Molecular Therapy 17(8): 1453-1464 (2009)). For example, peripheral blood is obtained 35-70 days after establishing leukemia in mice injected on day 21 with activatable cell surface receptor polypeptide T-cells, an equivalent number of mock-transduced T-cells, or no T-cells. Mice from each group are randomly bled for determination of peripheral blood CD19+ ALL blast counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). Briefly, assessment of activatable cell surface receptor polypeptide-mediated proliferation is performed in microtiter plates by mixing washed T-cells with K562 cells expressing CD19 (1(19) or CD32 and CD137 (KT32-BBL) for a final T-cell:K562 ratio of 2:1. K562 cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8+ T-cell expansion ex vivo. T-cells are enumerated in cultures using CountBright™ fluorescent beads (Invitrogen) and flow cytometry as described by the manufacturer. Activatable cell surface receptor polypeptide+ T-cells are identified by GFP expression using T-cells that are engineered with eGFP-2A linked activatable cell surface receptor polypeptide-expressing lentiviral vectors. For activatable cell surface receptor polypeptide+ T-cells not expressing GFP, the activatable cell surface receptor polypeptide+ T-cells are detected with biotinylated recombinant CD19 protein and a secondary avidin-PE conjugate. CD4+ and CD8+ expression on T-cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACS calibur flow cytometer, and data is analyzed according to the manufacturer's instructions.

Cytotoxicity can be assessed by a standard ⁵¹Cr-release assay (see, e.g., Milone et al., Molecular Therapy 17(8): 1453-1464 (2009)). Briefly, target cells (K562 lines and primary pro-B-ALL cells) are loaded with ⁵¹Cr (as NaCrO₄, New England Nuclear) at 37° C. for 2 hours with frequent agitation, washed twice in complete RPMI and plated into microtiter plates. Effector T-cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER-SR)/(TR-SR), where ER represents the average ⁵¹Cr released for each experimental condition.

Imaging technologies can be used to evaluate specific trafficking and proliferation of activatable cell surface receptor polypeptides in tumor-bearing animal models. Such assays have been described, e.g., in Barrett et al., Human Gene Therapy 22:1575-1586 (2011). Briefly, NOD/SCID/γc−/− (NSG) mice are injected IV with Nalm-6 cells followed 7 days later with T-cells 4 hour after electroporation with the activatable cell surface receptor polypeptide constructs. The T-cells are stably transfected with a lentiviral construct to express firefly luciferase, and mice are imaged for bioluminescence. Alternatively, therapeutic efficacy and specificity of a single injection of activatable cell surface receptor polypeptide+ T-cells in Nalm-6 xenograft model can be measured as the following: NSG mice are injected with Nalm-6 transduced to stably express firefly luciferase, followed by a single tail-vein injection of T-cells electroporated with a CD19 specific activatable cell surface receptor polypeptide 7 days later. Animals are imaged at various time points post injection. For example, photon-density heat maps of firefly luciferase positive leukemia in representative mice at day 5 (2 days before treatment) and day 8 (24 hours post activatable cell surface receptor polypeptide+PBLs) can be generated.

Other assays can also be used to evaluate the human target antigen activatable cell surface receptor polypeptide constructs of the disclosure.

Human Target Antigen Associated Diseases and/or Disorders

In one aspect, the disclosure provides methods for treating a disease associated with a human target antigen expression. In one aspect, the disclosure provides methods for treating a disease wherein part of the tumor is negative for a human target antigen and part of the tumor is positive for a human target antigen. For example, the activatable cell surface receptor polypeptide of the disclosure is useful for treating subjects that have undergone treatment for a disease associated with elevated expression of a human target antigen, wherein the subject that has undergone treatment for elevated levels of a human target antigen exhibits a disease associated with elevated levels of the human target antigen.

In one aspect, the disclosure pertains to a vector comprising anti-Human target antigen activatable cell surface receptor polypeptide operably linked to a promoter for expression in mammalian T-cells. In one aspect, the disclosure provides a recombinant T-cell expressing the Human target antigen activatable cell surface receptor polypeptide for use in treating CD19 or BCMA expressing tumors, wherein the recombinant T-cell expressing the human target antigen activatable cell surface receptor polypeptide is termed a human target antigen activatable cell surface receptor polypeptide-T cell. In one aspect, the human target antigen activatable cell surface receptor polypeptide-T cell of the disclosure is capable of contacting a tumor cell with at least one human target antigen activatable cell surface receptor polypeptide of the disclosure expressed on its surface such that the activatable cell surface receptor polypeptide-T cell targets the tumor cell and growth of the tumor is inhibited.

In one aspect, the disclosure pertains to a method of inhibiting growth of a CD19- or BCMA-expressing tumor cell, comprising contacting the tumor cell with a human target antigen activatable cell surface receptor polypeptide T-cell of the present disclosure such that the activatable cell surface receptor polypeptide-T is activated in response to the antigen and targets the cancer cell, wherein the growth of the tumor is inhibited.

In one aspect, the disclosure pertains to a method of treating cancer in a subject. The method comprises administering to the subject a human target antigen activatable cell surface receptor polypeptide T-cell of the present disclosure such that the cancer is treated in the subject. An example of a cancer that is treatable by a human target antigen activatable cell surface receptor polypeptide T-cell of the disclosure is a cancer associated with expression of the human target antigen. In one aspect, the cancer associated with expression of human target antigen is a hematological cancer. In one aspect, the hematological cancer is leukemia or lymphoma. In one aspect, a cancer associated with expression of CD19 includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of Human target antigen include, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with Human target antigen expression include, but not limited to, e.g., atypical and/or non-classical cancers, malignancies, precancerous conditions or proliferative diseases associated with expression of Human target antigen.

In some embodiments, a cancer that can be treated with a human target antigen activatable cell surface receptor polypeptide, e.g., as described herein, is multiple myeloma. Multiple myeloma is a cancer of the blood, characterized by accumulation of a plasma cell clone in the bone marrow. Current therapies for multiple myeloma include, but are not limited to, treatment with lenalidomide, which is an analog of thalidomide. Lenalidomide has activities which include anti-tumor activity, angiogenesis inhibition, and immunomodulation. Generally, myeloma cells are thought to be negative for Human target antigen expression by flow cytometry. The present disclosure encompasses the recognition that a small percent of myeloma tumor cells express human target antigen. Thus, in some embodiments, a CD19 or BCMA activatable cell surface receptor polypeptide, e.g., as described herein, may be used to target myeloma cells. In some embodiments, human target antigen activatable cell surface receptor polypeptide therapy can be used in combination with one or more additional therapies, e.g., lenalidomide treatment.

The disclosure includes a type of cellular therapy where T-cells are genetically modified to express an activatable cell surface receptor polypeptide and the activatable cell surface receptor polypeptide-expressing T-cell is infused to a recipient in need thereof. The infused cell is able to kill tumor cells in the recipient. Unlike antibody therapies, activatable cell surface receptor polypeptide-expressing T-cells are able to replicate in vivo resulting in long-term persistence that can lead to sustained tumor control. In various aspects, the T-cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T-cell to the patient.

The disclosure also includes a type of cellular therapy where T-cells are modified, e.g., by in vitro transcribed RNA, to transiently express an activatable cell surface receptor polypeptide and the activatable cell surface receptor polypeptide-expressing T-cell is infused to a recipient in need thereof. The infused cell is able to kill tumor cells in the recipient. Thus, in various aspects, the T-cells administered to the patient, is present for less than one month, e.g., three weeks, two weeks, or one week, after administration of the T-cell to the patient.

Without wishing to be bound by any particular theory, the anti-tumor immunity response elicited by the activatable cell surface receptor polypeptide-expressing T-cells may be an active or a passive immune response, or alternatively may be due to a direct vs indirect immune response. In one aspect, the activatable cell surface receptor polypeptide transduced T-cells exhibit specific proinflammatory cytokine secretion and potent cytolytic activity in response to human cancer cells expressing the Human target antigen, resist soluble Human target antigen inhibition, mediate bystander killing and mediate regression of an established human tumor. For example, antigen-less tumor cells within a heterogeneous field of CD19-expressing or BCMA-expressing tumor may be susceptible to indirect destruction by CD19-redirected or BCMA-redirectedT-cells that has previously reacted against adjacent antigen-positive cancer cells.

In one aspect, the human activatable cell surface receptor polypeptide-modified T-cells of the disclosure may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding an activatable cell surface receptor polypeptide to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing an activatable cell surface receptor polypeptide disclosed herein. The activatable cell surface receptor polypeptide-modified cell can be administered to a mammalian recipient to provide a therapeutic benefit. The mammalian recipient may be a human and the activatable cell surface receptor polypeptide-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present disclosure. Other suitable methods are known in the art, therefore the present disclosure is not limited to any particular method of ex vivo expansion of the cells. Briefly, ex vivo culture and expansion of T-cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the activatable cell surface receptor polypeptide-modified T-cells of the disclosure are used in the treatment of diseases, disorders and conditions associated with expression of a human target antigen. In certain aspects, the cells of the disclosure are used in the treatment of patients at risk for developing diseases, disorders and conditions associated with expression of Human target antigen. Thus, the present disclosure provides methods for the treatment or prevention of diseases, disorders and conditions associated with expression of a human target antigen comprising administering to a subject in need thereof, a therapeutically effective amount of the activatable cell surface receptor polypeptide-modified T-cells of the disclosure.

In one aspect the activatable cell surface receptor polypeptide-T-cells of the disclosures may be used to treat a proliferative disease such as a cancer or malignancy or is a precancerous condition such as a myelodysplasia, a myelodysplastic syndrome or a preleukemia. In one aspect, the cancer is a hematological cancer. In one aspect, the hematological cancer is leukemia or lymphoma. In one aspect, the activatable cell surface receptor polypeptide-T-cells of the disclosure may be used to treat cancers and malignancies such as, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with Human target antigen expression include, but not limited to, e.g., atypical and/or non-classical cancers, malignancies, precancerous conditions or proliferative diseases expressing Human target antigen. Non-cancer related indications associated with expression of Human target antigen include, but are not limited to, e.g., autoimmune disease, (e.g., lupus), inflammatory disorders (allergy and asthma) and transplantation.

The activatable cell surface receptor polypeptide-modified T-cells of the present disclosure may be administered either alone, or as a pharmaceutical composition in combination with diluents and/or with other components such as IL-2 or other cytokines or cell populations.

Hematologic Cancer

Hematological cancer conditions are the types of cancer such as leukemia and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CIVIL) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

The present disclosure provides for compositions and methods for treating cancer. In one aspect, the cancer is a hematologic cancer including but is not limited to hematological cancer is a leukemia or a lymphoma. In one aspect, the activatable cell surface receptor polypeptide-T-cells of the disclosure may be used to treat cancers and malignancies such as, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with CD19 expression includes, but not limited to, e.g., atypical and/or non-classical cancers, malignancies, precancerous conditions or proliferative diseases expressing CD19.

The present disclosure also provides methods for inhibiting the proliferation or reducing a CD19-expressing cell population, the methods comprising contacting a population of cells comprising a CD19-expressing cell with an anti-CD19 activatable cell surface receptor polypeptide-T-cell of the disclosure that binds to the CD19-expressing cell. In one aspect, the present disclosure provides methods for inhibiting the proliferation or reducing the population of cancer cells expressing CD19, the methods comprising contacting the CD19-expressing cancer cell population with an anti-CD19 activatable cell surface receptor polypeptide-T-cell of the disclosure that binds to the CD19-expressing cell. In certain aspects, the anti-CD19 activatable cell surface receptor polypeptide-T-cell of the disclosure reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with CD19-expressing cells relative to a negative control. In one aspect, the subject is a human.

The present disclosure also provides methods for preventing, treating and/or managing a disease associated with CD19-expressing cells (e.g., a hematologic cancer or atypical cancer expressing CD19), the methods comprising administering to a subject in need an anti-CD19 activatable cell surface receptor polypeptide-T-cell of the disclosure that binds to the CD19-expressing cell. In one aspect, the subject is a human. Non-limiting examples of disorders associated with CD19-expressing cells include autoimmune disorders (such as lupus), inflammatory disorders (such as allergies and asthma) and cancers (such as hematological cancers or atypical cancers expressing CD19).

The present disclosure also provides methods for preventing, treating and/or managing a disease associated with CD19-expressing cells, the methods comprising administering to a subject in need an anti-CD19 activatable cell surface receptor polypeptide-T-cell of the disclosure that binds to the CD19-expressing cell. In one aspect, the subject is a human.

The present disclosure provides methods for preventing relapse of cancer associated with CD19-expressing cells, the methods comprising administering to a subject in need thereof an anti-CD19 activatable cell surface receptor polypeptide-T-cell of the disclosure that binds to the CD19-expressing cell. In one aspect, the methods comprise administering to the subject in need thereof an effective amount of an anti-CD19 activatable cell surface receptor polypeptide-T-cell described herein that binds to the CD19-expressing cell in combination with an effective amount of another therapy.

Combination Therapies

An activatable cell surface receptor polypeptide-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes an activatable cell surface receptor polypeptide-expressing cell. Also provided are T-cells that express multiple activatable cell surface receptor polypeptides, which bind to the same or different target antigens, or same or different epitopes on the same target antigen. Also provided are populations of T-cells in which a first subset of T-cells express a first activatable cell surface receptor polypeptide and a second subset of T-cells express a second activatable cell surface receptor polypeptide.

An activatable cell surface receptor polypeptide-expressing cell described herein and the at least one additional therapeutic agent can be administered simultaneously, in the same or in separate compositions, or sequentially. For sequential administration, the activatable cell surface receptor polypeptide-expressing cell described herein can be administered first, and the additional agent can be administered second, or the order of administration can be reversed.

In further aspects, an activatable cell surface receptor polypeptide-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. peptide vaccine, such as that described in Izumoto et al. 2008 J Neurosurg 108:963-971.

In one embodiment, the subject can be administered an agent which reduces or ameliorates a side effect associated with the administration of an activatable cell surface receptor polypeptide-expressing cell. Side effects associated with the administration of an activatable cell surface receptor polypeptide-expressing cell include, but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods described herein can comprise administering an activatable cell surface receptor polypeptide-expressing cell described herein to a subject and further administering an agent to manage elevated levels of a soluble factor resulting from treatment with an activatable cell surface receptor polypeptide-expressing cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab (toc).

In one embodiment, the subject can be administered an agent which enhances the activity of an activatable cell surface receptor polypeptide-expressing cell. For example, in one embodiment, the agent can be an agent which inhibits an inhibitory molecule. Inhibitory molecules, e.g., Programmed Death 1 (PD1), can, in some embodiments, decrease the ability of an activatable cell surface receptor polypeptide-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize an activatable cell surface receptor polypeptide-expressing cell performance. In embodiments, an inhibitory nucleic acid, e.g., an inhibitory nucleic acid, e.g., a dsRNA, e.g., an siRNA or shRNA, can be used to inhibit expression of an inhibitory molecule in the activatable cell surface receptor polypeptide-expressing cell. In an embodiment the inhibitor is an shRNA. In an embodiment, the inhibitory molecule is inhibited within an activatable cell surface receptor polypeptide-expressing cell. In these embodiments, a dsRNA molecule that inhibits expression of the inhibitory molecule is linked to the nucleic acid that encodes a component, e.g., all of the components, of the activatable cell surface receptor polypeptide. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy™; Bristol-Myers Squibb; Tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the agent which enhances the activity of an activatable cell surface receptor polypeptide-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the activatable cell surface receptor polypeptide. In another embodiment, the fusion protein is expressed by a cell, e.g., a T-cell that does not express an anti-CD19 activatable cell surface receptor polypeptide.

Pharmaceutical Compositions

Pharmaceutical compositions of the present disclosure may comprise an activatable cell surface receptor polypeptide-expressing cell, e.g., a plurality of activatable cell surface receptor polypeptide-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T-cells described herein may be administered at a dosage of 10⁴ to 10⁹ cells/kg body weight, in some instances 10⁵ to 10⁶ cells/kg body weight, including all integer values within those ranges. T-cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T-cells to a subject and then subsequently redraw blood (or have an apheresis performed), activate T-cells therefrom according to the present disclosure, and reinfuse the patient with these activated and expanded T-cells. This process can be carried out multiple times every few weeks. In certain aspects, T-cells can be activated from blood draws of from 10 cc to 400 cc. In certain aspects, T-cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one aspect, the T-cell compositions of the present disclosure are administered to a patient by intradermal or subcutaneous injection. In one aspect, the T-cell compositions of the present disclosure are administered by i.v. injection. The compositions of T-cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T-cells. These T-cell isolates may be expanded by methods known in the art and treated such that one or more activatable cell surface receptor polypeptide constructs of the disclosure may be introduced, thereby creating an activatable cell surface receptor polypeptide-expressing T-cell of the disclosure. Subjects in need thereof may subsequently undergo standard treatment with high dose chemotherapy followed by peripheral blood stem cell transplantation. In certain aspects, following or concurrent with the transplant, subjects receive an infusion of the expanded activatable cell surface receptor polypeptide T-cells of the present disclosure. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the activatable cell surface receptor polypeptide is introduced into T-cells, e.g., using in vitro transcription, and the subject (e.g., human) receives an initial administration of activatable cell surface receptor polypeptide T-cells of the disclosure, and one or more subsequent administrations of the activatable cell surface receptor polypeptide T-cells of the disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the activatable cell surface receptor polypeptide T-cells of the disclosure are administered to the subject (e.g., human) per week, e.g., 2, 3, or 4 administrations of the activatable cell surface receptor polypeptide T-cells of the disclosure are administered per week. In one embodiment, the subject (e.g., human subject) receives more than one administration of the activatable cell surface receptor polypeptide T-cells per week (e.g., 2, 3 or 4 administrations per week) (also referred to herein as a cycle), followed by a week of no activatable cell surface receptor polypeptide T-cells administrations, and then one or more additional administration of the activatable cell surface receptor polypeptide T-cells (e.g., more than one administration of the activatable cell surface receptor polypeptide T-cells per week) is administered to the subject. In another embodiment, the subject (e.g., human subject) receives more than one cycle of activatable cell surface receptor polypeptide T-cells, and the time between each cycle is less than 10, 9, 8, 7, 6, 5, 4, or 3 days. In one embodiment, the activatable cell surface receptor polypeptide T-cells are administered every other day for 3 administrations per week. In one embodiment, the activatable cell surface receptor polypeptide T-cells of the disclosure are administered for at least two, three, four, five, six, seven, eight or more weeks.

In one aspect, CD19 activatable cell surface receptor polypeptide T-cells are generated using lentiviral viral vectors, such as lentivirus. Activatable cell surface receptor polypeptide-T-cells generated that way will have stable activatable cell surface receptor polypeptide expression.

In one aspect, activatable cell surface receptor polypeptide T-cells transiently express activatable cell surface receptor polypeptide vectors for 4, 5, 6, 7, 8, 9, 10, 11, 12, 13, 14, 15 days after transduction. Transient expression of activatable cell surface receptor polypeptides can be effected by RNA activatable cell surface receptor polypeptide vector delivery. In one aspect, the activatable cell surface receptor polypeptide RNA is transduced into the T-cell by electroporation.

A potential issue that can arise in patients being treated using transiently expressing activatable cell surface receptor polypeptide T-cells (particularly with murine scFv bearing activatable cell surface receptor polypeptide T-cells) is anaphylaxis after multiple treatments.

Without being bound by this theory, it is believed that such an anaphylactic response might be caused by a patient developing humoral anti-activatable cell surface receptor polypeptide response, i.e., anti-activatable cell surface receptor polypeptide antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-activatable cell surface receptor polypeptide antibody response during the course of transient activatable cell surface receptor polypeptide therapy (such as those generated by RNA transductions), activatable cell surface receptor polypeptide T-cell infusion breaks should not last more than ten to fourteen days.

### EXAMPLES

The disclosure is further described in detail by reference to the following exemplary polypeptides. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present disclosure and practice the claimed methods. The following working examples specifically point out various aspects of the present disclosure, and are not to be construed as limiting in any way the remainder of the disclosure.

### Example 1. An Exemplary Activatable Cell Surface Receptor Polypeptide

The activatable cell surface receptor polypeptides describe herein, comprising a TCR complex or a CAR, typically comprises an extracellular domain, a transmembrane domain, and an intracellular domain. In an exemplary embodiment, the polypeptide comprises a TCR complex which is a human TCR complex comprising a CD3-epsilon polypeptide, a CD3-gamma polypeptide, a CD3-delta polypeptide, a CD3-zeta polypeptide, a TCR alpha chain polypeptide and a TCR beta chain polypeptide. The human CD3-epsilon polypeptide canonical sequence is Uniprot Accession No. P07766 (SEQ ID NO: 110). The human CD3-gamma polypeptide canonical sequence is Uniprot Accession No. P09693 (SEQ ID NO: 111). The human CD3-delta polypeptide canonical sequence is Uniprot Accession No. P043234 (SEQ ID NO: 112). The human CD3-zeta polypeptide canonical sequence is Uniprot Accession No. P20963 (SEQ ID NO: 113). The human TCR alpha chain canonical sequence is Uniprot Accession No. Q6ISU1 (SEQ ID NO: 114). The human TCR beta chain C region canonical sequence is Uniprot Accession No. P01850 (SEQ ID NO: 117), a human TCR beta chain V region sequence is P04435 (SEQ ID NO: 118).

The human CD3-epsilon polypeptide canonical sequence is:

(SEQ ID NO: 110)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI.

The human CD3-gamma polypeptide canonical sequence is:

(SEQ ID NO: 111)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN.

The human CD3-delta polypeptide canonical sequence is:

(SEQ ID NO: 112)
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK.

The human CD3-zeta polypeptide canonical sequence is:

(SEQ ID NO: 113)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR.

The human TCR alpha chain canonical sequence is:

(SEQ ID NO: 114)
MAGTWLLLLLALGCPALPTGVGGTPFPSLAPPIMLLVDGKQQMVVVCLVL
DVAPPGLDSPIWFSAGNGSALDAFTYGPSPATDGTWTNLAHLSLPSEELA
SWEPLVCHTGPGAEGHSRSTQPMHLSGEASTARTCPQEPLRGTPGGALWL
GVLRLLLFKLLLFDLLLTCSCLCDPAGPLPSPATTTRLRALGSHRLHPAT
ETGGREATSSPRPQPRDRRWGDTPPGRKPGSPVWGEGSYLSSYPTCPAQA
WCSRSALRAPSSSLGAFFAGDLPPPLQAGAA.

The human TCR alpha chain C region canonical sequence is:

(SEQ ID NO: 115)
PNIQNPDPAVYQLRDSKSSDKSVCLFTDFDSQTNVSQSKDSDVYITDKTV
LDMRSMDFKSNSAVAWSNKSDFACANAFNNSIIPEDTFFPSPESSCDVKL
VEKSFETDTNLNFQNLSVIGFRILLLKVAGFNLLMTLRLWSS.

The human TCR alpha chain V region CTL-L17 canonical sequence is:

(SEQ ID NO: 116)
MAMLLGASVLILWLQPDWVNSQQKNDDQQVKQNSPSLSVQEGRISILNCD
YTNSMFDYFLWYKKYPAEGPTFLISISSIKDKNEDGRFTVFLNKSAKHLS
LHIVPSQPGDSAVYFCAAKGAGTASKLTFGTGTRLQVTL.

The human TCR beta chain C region canonical sequence is:

(SEQ ID NO: 117)
EDLNKVFPPEVAVFEPSEAEISHTQKATLVCLATGFFPDHVELSWWVNGK
EVHSGVSTDPQPLKEQPALNDSRYCLSSRLRVSATFWQNPRNHFRCQVQF
YGLSENDEWTQDRAKPVTQIVSAEAWGRADCGFTSVSYQQGVLSATILYE
ILLGKATLYAVLVSALVLMAMVKRKDF.

The human TCR beta chain V region CTL-L17 canonical sequence is:

(SEQ ID NO: 118)
MGTSLLCWMALCLLGADHADTGVSQNPRHNITKRGQNVTFRCDPISEHNR
LYWYRQTLGQGPEFLTYFQNEAQLEKSRLLSDRFSAERPKGSFSTLEIQR
TEQGDSAMYLCASSLAGLNQPQHFGDGTRLSIL.

The human TCR beta chain V region YT35 canonical sequence is:

(SEQ ID NO: 119)
MDSWTFCCVSLCILVAKHTDAGVIQSPRHEVTEMGQEVTLRCKPISGHNS
LFWYRQTMMRGLELLIYFNNNVPIDDSGMPEDRFSAKMPNASFSTLKIQP
SEPRDSAVYFCASSFSTCSANYGYTFGSGTRLTVV.

### Example 2. Reconstitution of EGFR-Binding Activity of Cetuximab scFv by Co-Expression of Vh CAR and Vl CAR

Expression of CAR Constructs in Expi293 Cells

CAR constructs were cloned into the lentiviral vector pLV09 using standard molecular biology techniques. Vectors encoding CAR constructs were then transfected into Expi293 ™ cells using Expifectamine reagent according to the manufacturer's recommendations. Briefly, 0.85 mL cells were seeded in a deep well 96 well plate at a density of 2×10⁶ cells/mL. 1 ug CAR plasmid DNA was added to 50 uL RPMI and set aside. 2.7 uL Expifectamine reagent was added to 50 uL RPMI. After 5 minutes, the DNA-containing solution was added to the Expifectamine/RPMI and incubated at room temperature. After 20 minutes, the transfection mix was added drop wise to cells. 24 hours after transfection enhancer solutions were added. Cells were analyzed by flow cytometry 48 hours post-transfection.

Binding Assays

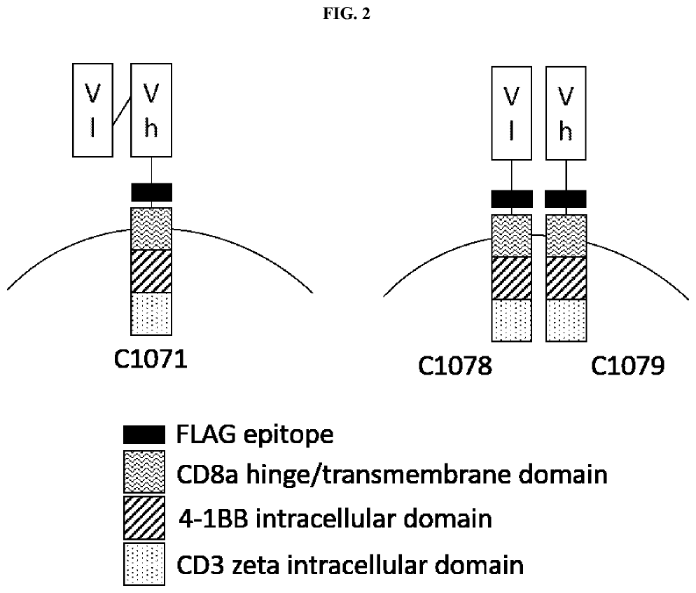

Expi293 cells were transfected with pUC18 vector (negative control), C1071 (SEQ ID NO: 5), C1078 (SEQ ID NO: 6) alone, C1079 (SEQ ID NO: 7) alone, or both C1078 and C1079 (expressed on separate vectors), using the methods described above. FIG. 2 shows a diagram of the constructs used. Cetuximab VH (SEQ ID NO: 124) and VL (SEQ ID NO. 125) were used in these constructs. Transfected cells were incubated with Fc-tagged EGFR extracellular domain (ECD). Cells were then washed to remove unbound protein. Cells were then incubated with secondary antibody, capable of recognizing human Fc, conjugated to Dylight 650. Binding of the secondary antibody to cells was measured by flow cytometry.

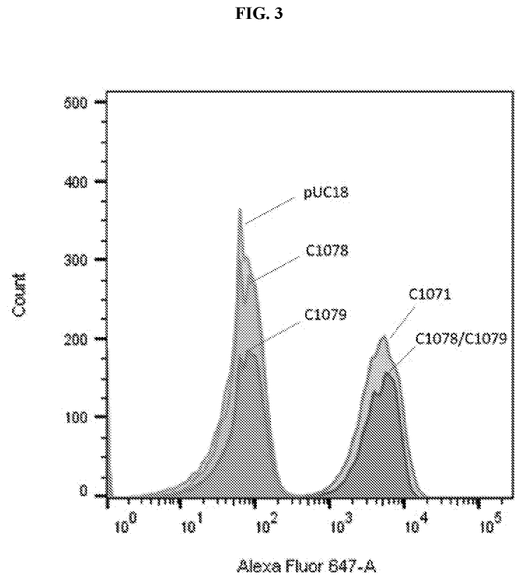

Results are shown in FIG. 3. No binding of Fc-tagged EGFR ECD was observed for the cells expressing C1078 or C1079 alone. Binding was observed at comparable levels for cells expressing C1071 and cells both expressing C1078 and C1079 together.

### Example 3. Reconstitution of EGFR-Binding Activity of Cetuximab scFv by Co-Expression of Vh CAR and Vl CAR Each Comprising an Extracellular Dimerization Domain

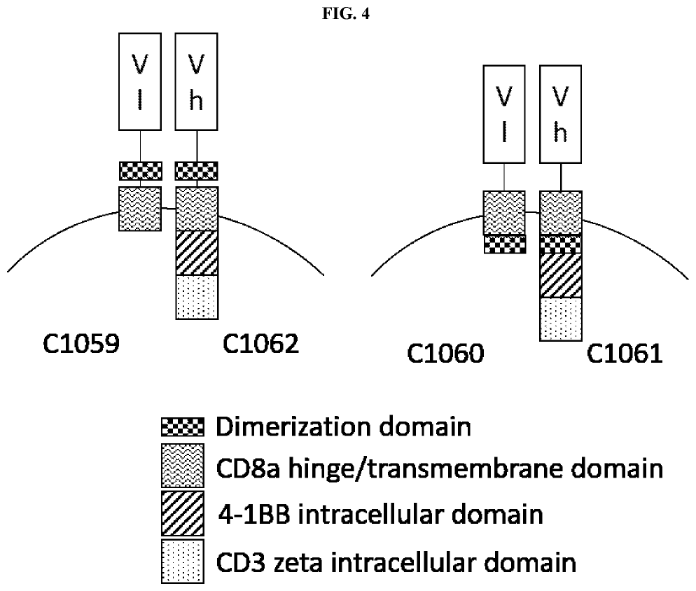

Expi293 cells were transfected with pUC18 vector (negative control), C1059 (SEQ ID NO: 1) alone, C1062 (SEQ ID NO: 4) alone, or both C1059 and C1062 (expressed on separate vectors), using the methods described in Example 2. FIG. 4 shows a diagram of the constructs used. Cetuximab VH (SEQ ID NO: 124) and VL (SEQ ID NO. 125) were used in these constructs. Transfected cells were incubated with Fc-tagged EGFR extracellular domain (ECD). Cells were then washed to remove unbound protein. Cells were then incubated with secondary antibody, capable of recognizing human Fc, conjugated to Dylight 650. Binding of the secondary antibody to cells was measured by flow cytometry.

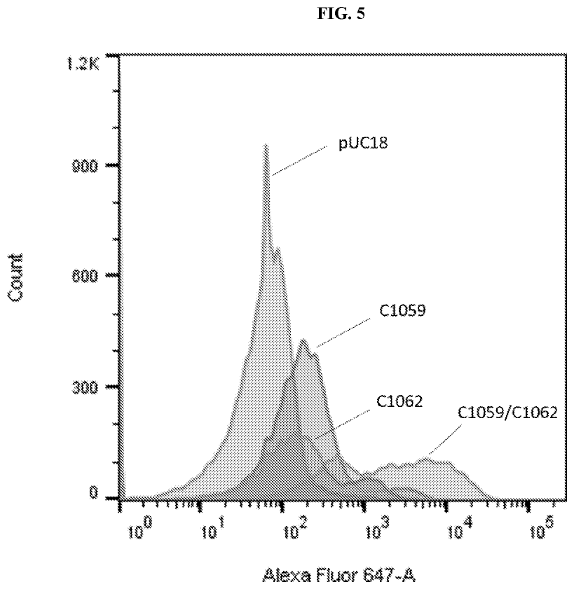

Results are shown in FIG. 5. Little binding of Fc-tagged EGFR ECD was observed for the cells expressing C1059 or C1062 alone. High levels of binding were observed for cells expressing C1059 and C1062 together.

### Example 4. Reconstitution of EGFR-Binding Activity of Cetuximab scFv by Co-Expression of Vh CAR and Vl CAR Each Comprising an Intracellular Dimerization Domain

Expi293 cells were transfected with pUC18 vector (negative control), C1060 (SEQ ID NO: 2) alone, C1061 (SEQ ID NO: 3) alone, or both C1060 and C1061 (expressed on separate vectors), using the methods described in Example 2. FIG. 4 shows a diagram of the constructs used. Cetuximab VH (SEQ ID NO: 124) and VL (SEQ ID NO. 125) were used in these constructs. Transfected cells were incubated with Fc-tagged EGFR extracellular domain (ECD). Cells were then washed to remove unbound protein. Cells were then incubated with secondary antibody, capable of recognizing human Fc, conjugated to Dylight 650. Binding of the secondary antibody to cells was measured by flow cytometry.

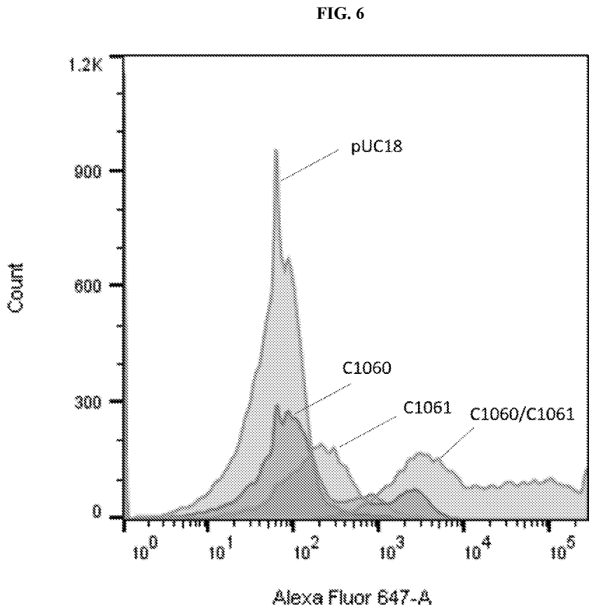

Results are shown in FIG. 6. Little binding of Fc-tagged EGFR ECD was observed for the cells expressing C1060 or C1061 alone. High levels of binding were observed for cells expressing C1060 and C1061 together.

### Example 5. Activation of Antigen Binding Activity of a CAR Expressing an Activatable Receptor by Trypsin Treatment

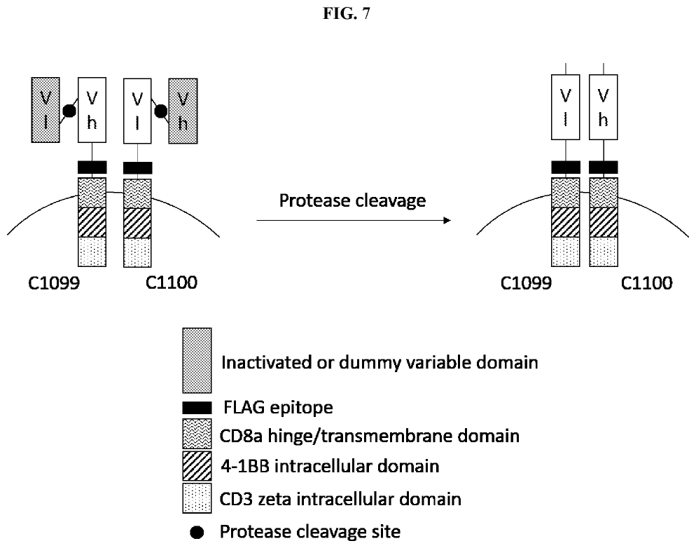

Expi293 cells were transfected with pUC18 vector (negative control), or both C1099 (SEQ ID NO: 9) and C1100 (SEQ ID NO: 10) together (expressed on separate vectors), using the methods described in Example 2. FIG. 7 shows a diagram of the constructs used. Cetuximab VH (SEQ ID NO: 124) and VL (SEQ ID NO: 125) were used in these constructs. Mutated, inactivated (“dummy”) cexutimab VH (SEQ ID NO: 126) and VL (SEQ ID NO: 127) were also used in these constructs. Transfected cells were treated briefly with trypsin or PBS. Cells were then incubated with Fc-tagged EGFR extracellular domain (ECD). Next, cells were washed to remove unbound protein. Cells were then incubated with secondary antibody, capable of recognizing human Fc, conjugated to Dylight 650. Binding of the secondary antibody to cells was measured by flow cytometry. Cells were grouped into low expressors, medium expressors, and high expressors by measuring anti-FLAG antibody staining and a secondary antibody labeled with DyLight 405.

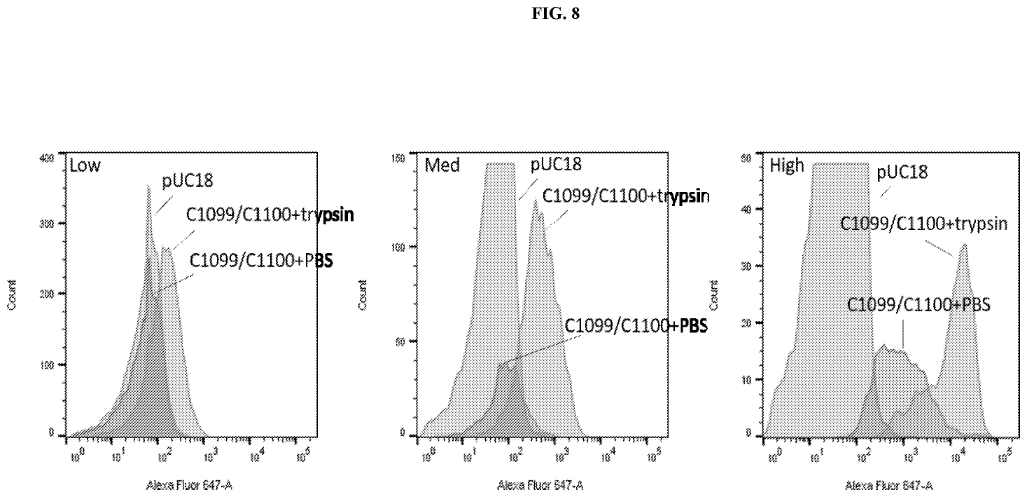

Results are shown in FIG. 8 for the low, medium, and high expressors, as indicated. Little binding of Fc-tagged EGFR ECD was observed for the cells expressing C1099 and C1100 treated with PBS. High levels of binding were observed for cells expressing C1099 and C1100 when treated with trypsin.

### Example 6. Activation of Antigen Binding Activity of a CAR Expressing an Activatable Receptor by Matriptase (ST14) Treatment

Expi293 cells were transfected with pUC18 vector (negative control), or both C1099 (SEQ ID NO: 9) and C1100 (SEQ ID NO: 10) together (expressed on separate vectors), using the methods described in Example 2. FIG. 7 shows a diagram of the constructs used. Cetuximab VH (SEQ ID NO: 124) and VL (SEQ ID NO: 125) were used in these constructs. Mutated, inactivated (“dummy”) cexutimab VH (SEQ ID NO: 126) and VL (SEQ ID NO: 127) were also used in these constructs. Transfected cells were treated briefly with 1 μg/mL matriptase (ST14) or PBS. Cells were then incubated with Fc-tagged EGFR extracellular domain (ECD). Next, cells were washed to remove unbound protein. Cells were then incubated with secondary antibody, capable of recognizing human Fc, conjugated to Dylight 650. Binding of the secondary antibody to cells was measured by flow cytometry. Cells were grouped into low expressors and medium expressors by measuring anti-FLAG antibody staining and a secondary antibody labeled with DyLight 405.

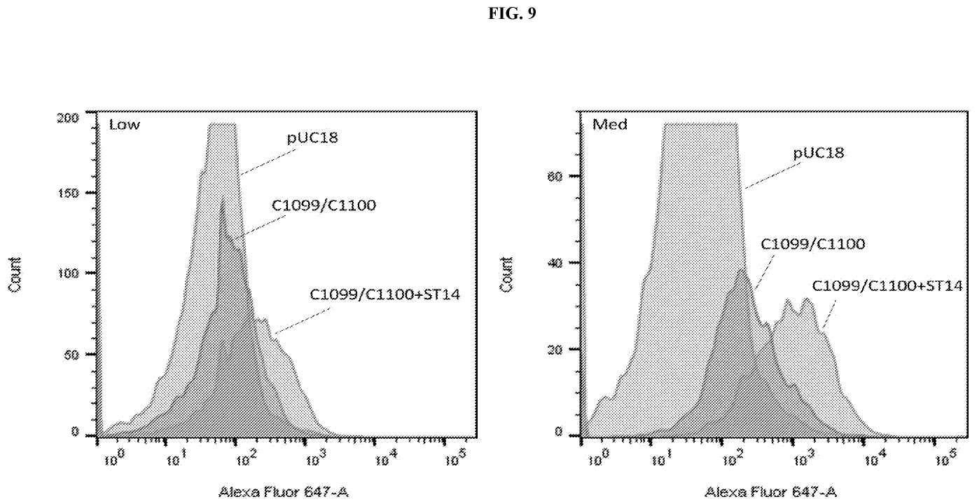

Results are shown in FIG. 9 for the low and medium expressors, as indicated. Little binding of Fc-tagged EGFR ECD was observed for the cells expressing C1099 and C1100 treated with PBS. High levels of binding were observed for cells expressing C1099 and C1100 when treated with matriptase (ST14).

### Example 7. Disruption of Antigen-Binding Activity of Fabs by Pairing One Targeting Variable Domain with a Non-Targeting Variable Domain

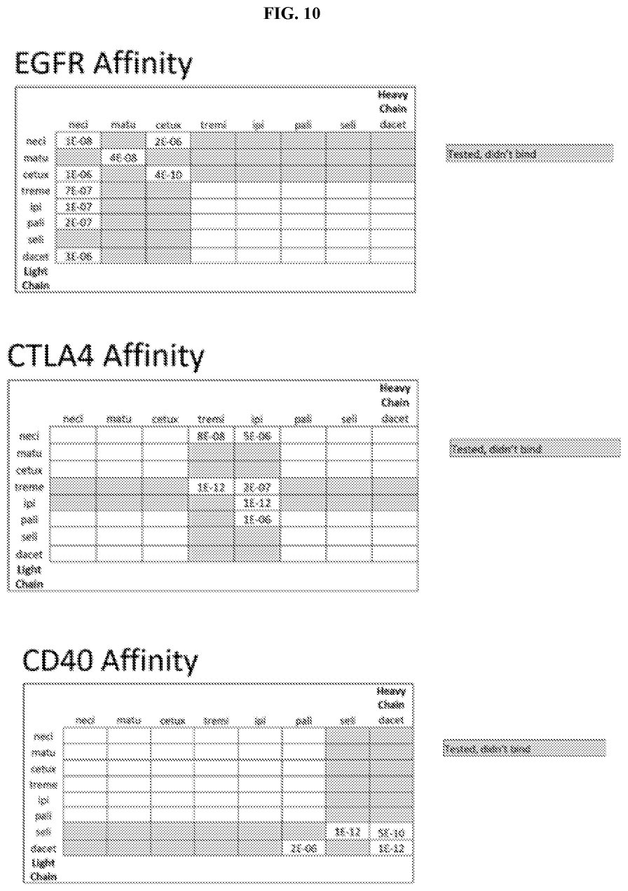

Expi293 cells were transfected with plasmids encoding the light chain from the antibody indicated in each row and the heavy chain from the antibody indicated in each column of FIG. 10, thereby generating Fabs. Antibodies from which light and heavy chains were used are: necitumumab (“neci”; anti-EGFR antibody), matuzumab (“matu”; anti-EGFR antibody), cetuximab (“cetux”; anti-EGFR antibody), tremelimumab (“tremi” and “treme”; anti-CTLA4 antibody), ipilimumab (“ipi”; anti-CTLA4 antibody), palivizumab (“pali”; anti-CTLA4 antibody), selicrelumab (“seli”; anti-CD40 antibody), and dacetuzumab (“dacet”; anti-CD40 antibody). Fabs were purified from the Expi293 cells, and the affinity of each Fab for EGFR, CTLA4, or CD40 was determined using bio-layer interferometry measurements on an Octet® system. Results are shown in FIG. 10. Empty white wells indicate untested combinations. Numbers indicate Kd. Gray cells indicate that a combination was tested, but no binding was observed.

Results demonstrate that pairing of a given heavy chain or light chain with a light chain or heavy chain, respectively, from a different antibody results in a loss of target antigen binding.

### Example 8. Cancer Cell Killing Activity of CAR T Cells Expressing Activatable Receptors

Expression of CAR Constructs in Primary Human T Cells

Freshly thawed T cells were activated using anti-CD3/anti-CD28 antibody coated beads. After 24 hours, IL-2 was added to 100 U/mL. Three days later 500,000 activated T cells were infected with 0.5 mL lentiviral supernatant containing 8 ug/mL polybrene by centrifugation at 800×g for 1 hour at 32° C. Following centrifugation, the viral supernatant was removed and the cells were grown in standard T cell expansion medium. CAR expression was analyzed by flow cytometry three days post-infection.

Cell Killing Assays

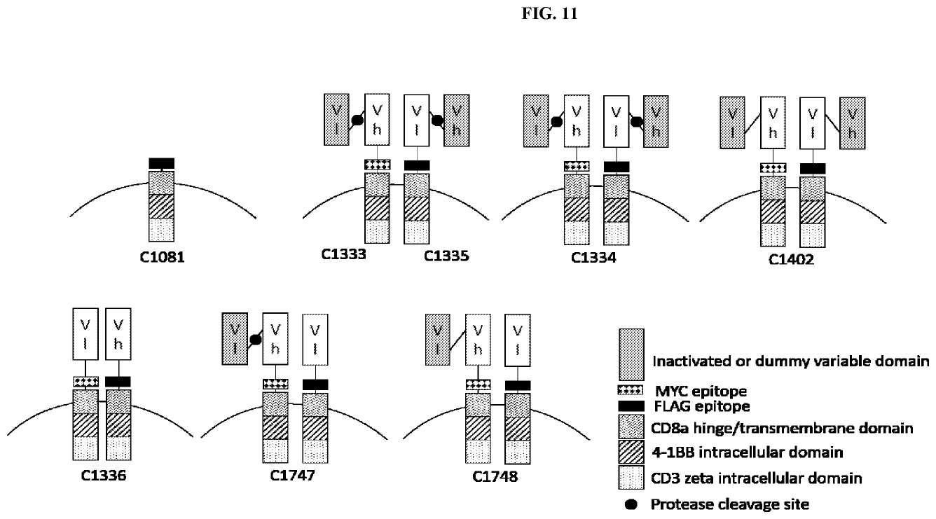

CAR T cells were generated expressing C1081 (SEQ ID NO: 8), C1333 (SEQ ID NO: 11) alone, C1335 (SEQ ID NO: 13) alone, both C1333 and C1335 (expressed on separate vectors), C1334 (SEQ ID NO: 12), C1402 (SEQ ID NO:15), C1336 (SEQ ID NO: 14), C1747 (SEQ ID NO: 23), or C1748 (SEQ ID NO: 24) as described above. FIG. 11 shows a diagram of the constructs used. Panitumumab VH (SEQ ID NO: 128) and VL (SEQ ID NO: 129) were used in constructs as indicated. “Dummy” palivizumab VH (SEQ ID NO: 130) and VL (SEQ ID NO: 131) were also used in these constructs, where indicated.

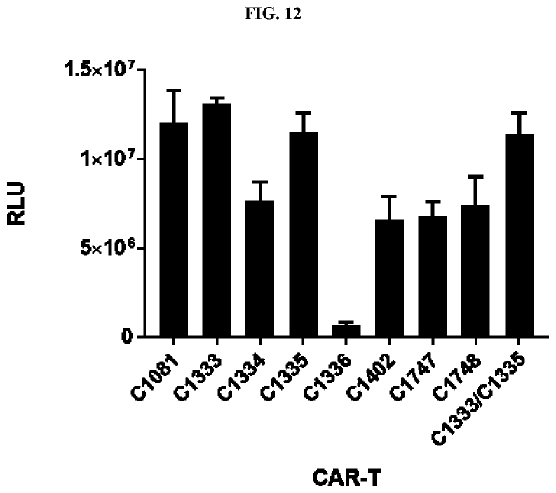

CAR T cells were mixed at a 10:1 ratio with EGFR-expressing HCT116 cells engineered to express luciferase. Luciferase activity was measured 72 hours later as a readout of HCT116 viability. Results are shown in FIG. 12. High cell viability was observed for cells treated with CAR T cells expressing C1081, which contains no targeting domain. Low cell viability (i.e., high cell killing) was observed for cells treated with CAR T cells expressing C1336, which contains VH and VL targeting domains and lacks inactive or “dummy” domains.

### Example 9. Vh/V1 Dimer Interface Engineering Reduces Background Cell Killing Activity of CAR T Cells Expressing Activatable Receptors

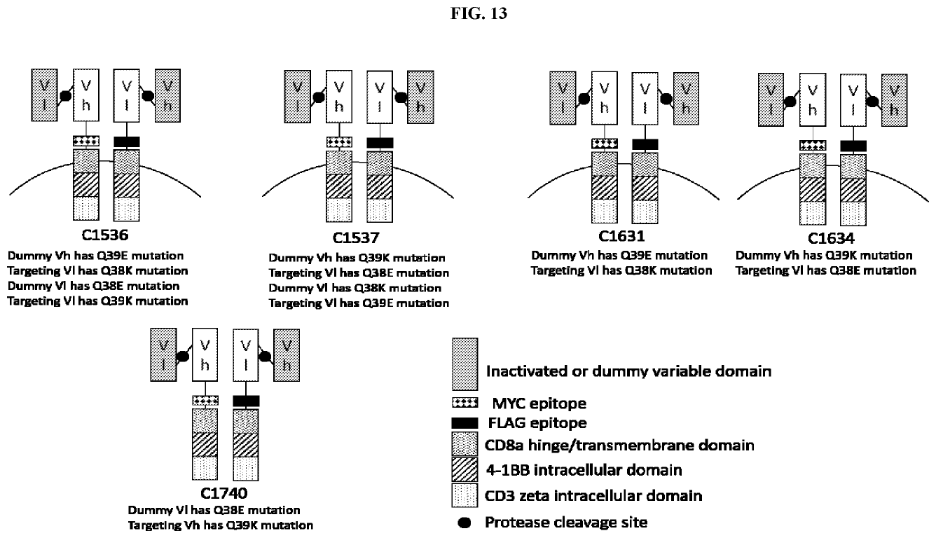

CAR T cells were generated expressing C1081, C1334, C1536 (SEQ ID NO: 19), C1537 (SEQ ID NO: 20), C1631 (SEQ ID NO: 21), C1634 (SEQ ID NO: 145), or C1740 (SEQ ID NO: 22), as described in Example 8. FIG. 13 and FIG. 11 show diagrams of the constructs used. Mutated panitumumab VH (SEQ ID NOS: 135 and 139) and VL (SEQ ID NOS: 133 and 137) were used in constructs as indicated, except C1334, where wild type panitumumab VH (SEQ ID NO: 128) and VL (SEQ ID NO: 129) were used. Mutated “dummy” palivizumab VH (SEQ ID NO: 132 and 136) and VL (SEQ ID NO: 134 and 138) were also used in constructs as indicated, except C1334, where wild type “dummy” palivizumab VH (SEQ ID NO: 130) and VL (SEQ ID NO: 131) were used. The mutated domains were engineered such that a salt bridge was formed between the VH and dummy VL, and between the VL and dummy VH, using the indicated mutations in FIG. 13.

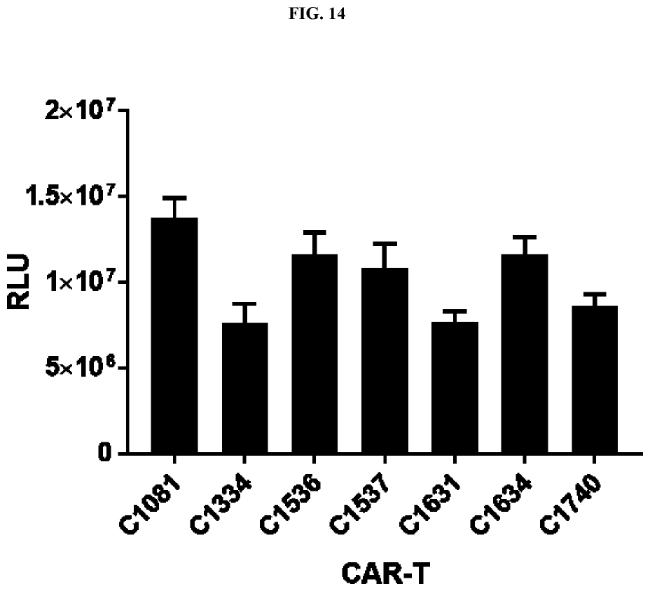

CAR T cells were mixed at a 10:1 ratio with EGFR-expressing HCT116 cells engineered to express luciferase. Luciferase activity was measured 72 hours later as a readout of HCT116 viability. Results are shown in FIG. 14. Results demonstrate low levels of background cell killing when treating with CAR T cells expressing mutated, engineered targeting domains which form a salt bridge.

### Example 10. Removal of CAR Signaling Modules from One Chain Reduces Background Cell Killing Activity of CAR T Cells Expressing Activatable Receptors

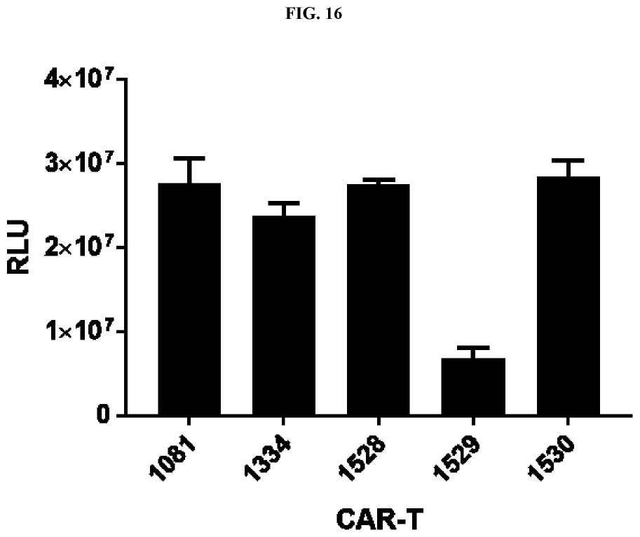

CAR T cells were generated expressing C1081 (SEQ ID NO: 8), C1334 (SEQ ID NO: 12), C1528 (SEQ ID NO: 16), C1529 (SEQ ID NO: 17), or C1530 (SEQ ID NO: 18), as described in Example 8. FIG. 16 and FIG. 11 show diagrams of the constructs used. Panitumumab VH (SEQ ID NO: 128) and VL (SEQ ID NO: 129) were used in constructs as indicated. “Dummy” palivizumab VH (SEQ ID NO: 130) and VL (SEQ ID NO: 131) were also used in these constructs, where indicated.

CAR T cells were mixed at a 10:1 ratio with EGFR-expressing HCT116 cells engineered to express luciferase. Luciferase activity was measured 72 hours later as a readout of HCT116 viability. Results are shown in FIG. 16. Low levels of cell viability (e.g., high levels of cell killing) are observed in cells treated with CAR T cells expressing C1529. Little to no background cell killing is observed when treating with CART cells expressing C1528 or C1530.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Sequence Table

| | | |
|---|---|---|
| SEQ ID | C1059 | MALPVTALLLPLALLLHAARPQVQLKQSGPGLVQPSQSLSITCTVSGFSL |
| NO: 1 | | TNYGVHWVRQSPGKGLEWLGVIWSGGNTDYNTPFTSRLSINKDNSKSQ |
| | | VFFKMNSLQSNDTAIYYCARALTYYDYEFAYWGQGTLVTVSATTTPAPR |
| | | PPTPAPTIASQPLSLRPEACRPAAGGAVHTRGLDFACDIYIWAPLAGTCG |
| | | VLLLSLVITLYKVSALKEKVSALKEKVSALKEKVSALKEKVSALKE |
| SEQ ID | C1060 | MALPVTALLLPLALLLHAARPQVQLKQSGPGLVQPSQSLSITCTVSGFSL |
| NO: 2 | | TNYGVHWVRQSPGKGLEWLGVIWSGGNTDYNTPFTSRLSINKDNSKSQ |
| | | VFFKMNSLQSNDTAIYYCARALTYYDYEFAYWGQGTLVTVSASGGGGS |
| | | KVSALKEKVSALKEKVSALKEKVSALKEKVSALKETTTPAPRPPTPAPTI |
| | | ASQPLSLRPEACRPAAGGAVHTRGLDFACDIYIWAPLAGTCGVLLLSLVI |
| | | TLY |
| SEQ ID | C1061 | MALPVTALLLPLALLLHAARPDILLTQSPVILSVSPGERVSFSCRASQSIGT |
| NO: 3 | | NIHWYQQRTNGSPRLLIKYASESISGIPSRFSGSGSGTDFTLSINSVESEDIA |
| | | DYYCQQNNNWPTTFGAGTKLELKGGGGSEVSALEKEVSALEKEVSALE |
| | | KEVSALEKEVSALEKTTTPAPRPPTPAPTIASQPLSLRPEACRPAAGGAVH |
| | | TRGLDFACDIYIWAPLAGTCGVLLLSLVITLYKRGRKKLLYIFKQPFMRP |
| | | VQTTQEEDGCSCRFPEEEEGGCELRVKFSRSADAPAYQQGQNQLYNELN |
| | | LGRREEYDVLDKRRGRDPEMGGKPRRKNPQEGLYNELQKDKMAEAYS |
| | | EIGMKGERRRGKGHDGLYQGLSTATKDTYDALHMQALPPR |
| SEQ ID | C1062 | MALPVTALLLPLALLLHAARPDILLTQSPVILSVSPGERVSFSCRASQSIGT |
| NO: 4 | | NIHWYQQRTNGSPRLLIKYASESISGIPSRFSGSGSGTDFTLSINSVESEDIA |
| | | DYYCQQNNNWPTTFGAGTKLELKTTTPAPRPPTPAPTIASQPLSLRPEAC |
| | | RPAAGGAVHTRGLDFACDIYIWAPLAGTCGVLLLSLVITLYEVSALEKEV |
| | | SALEKEVSALEKEVSALEKEVSALEKKRGRKKLLYIFKQPFMRPVQTTQE |
| | | EDGCSCRFPEEEEGGCELRVKFSRSADAPAYQQGQNQLYNELNLGRREE |
| | | YDVLDKRRGRDPEMGGKPRRKNPQEGLYNELQKDKMAEAYSEIGMKG |
| | | ERRRGKGHDGLYQGLSTATKDTYDALHMQALPPR |
| SEQ ID | C1071 | MALPVTALLLPLALLLHAARPDILLTQSPVILSVSPGERVSFSCRASQSIGT |
| NO: 5 | | NIHWYQQRTNGSPRLLIKYASESISGIPSRFSGSGSGTDFTLSINSVESEDIA |
| | | DYYCQQNNNWPTTFGAGTKLELKGGGGSGGGGSGGGGSQVQLKQSGP |
| | | GLVQPSQSLSITCTVSGFSLTNYGVHWVRQSPGKGLEWLGVIWSGGNTD |
| | | YNTPFTSRLSINKDNSKSQVFFKMNSLQSNDTAIYYCARALTYYDYEFAY |
| | | WGQGTLVTVSATSDYKDDDDKTTTPAPRPPTPAPTIASQPLSLRPEACRP |
| | | AAGGAVHTRGLDFACDIYIWAPLAGTCGVLLLSLVITLYKRGRKKLLYIF |
| | | KQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| SEQ ID | C1078 | MALPVTALLLPLALLLHAARPQVQLKQSGPGLVQPSQSLSITCTVSGFSL |
| NO: 6 | | TNYGVHWVRQSPGKGLEWLGVIWSGGNTDYNTPFTSRLSINKDNSKSQ |
| | | VFFKMNSLQSNDTAIYYCARALTYYDYEFAYWGQGTLVTVSATSDYKD |
| | | DDDKTTTPAPRPPTPAPTIASQPLSLRPEACRPAAGGAVHTRGLDFACDIY |
| | | IWAPLAGTCGVLLLSLVITLYKRGRKKLLYIFKQPFMRPVQTTQEEDGCS |
| | | CRFPEEEEGGCELRVKFSRSADAPAYQQGQNQLYNELNLGRREEYDVLD |
| | | KRRGRDPEMGGKPRRKNPQEGLYNELQKDKMAEAYSEIGMKGERRRG |
| | | KGHDGLYQGLSTATKDTYDALHMQALPPR |
| SEQ ID | C1079 | MALPVTALLLPLALLLHAARPDILLTQSPVILSVSPGERVSFSCRASQSIGT |
| NO: 7 | | NIHWYQQRTNGSPRLLIKYASESISGIPSRFSGSGSGTDFTLSINSVESEDIA |
| | | DYYCQQNNNWPTTFGAGTKLELKTSDYKDDDDKTTTPAPRPPTPAPTIA |
| | | SQPLSLRPEACRPAAGGAVHTRGLDFACDIYIWAPLAGTCGVLLLSLVIT |
| | | LYKRGRKKLLYIFKQPFMRPVQTTQEEDGCSCRFPEEEEGGCELRVKFSR |
| | | SADAPAYQQGQNQLYNELNLGRREEYDVLDKRRGRDPEMGGKPRRKN |
| | | PQEGLYNELQKDKMAEAYSEIGMKGERRRGKGHDGLYQGLSTATKDTY |
| | | DALHMQALPPR |
| SEQ ID | C1081 | MALPVTALLLPLALLLHAARPTSDYKDDDDKTTTPAPRPPTPAPTIASQP |
| NO: 8 | | LSLRPEACRPAAGGAVHTRGLDFACDIYIWAPLAGTCGVLLLSLVITLYK |
| | | RGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDA |
| | | LHMQALPPR |
| SEQ ID | C1099 | MALPVTALLLPLALLLHAARPQVQLKQSGPGLVQPSQSLSITCTVSGFSL |
| NO: 9 | | TSYGVHWVRQSPGKGLEWLGVIASGGSTDYNTPFTSRLSINKDNSKSQV |
| | | FFKMNSLQSNDTAIYYCARALTAARAEFAYWGQGTLVTVSAGSSGGSG |
| | | GSGGSGLSGRSDNHGSSGTDILLTQSPVILSVSPGERVSFSCRASQSIGTNI |
| | | HWYQQRTNGSPRLLIKYASESISGIPSRFSGSGSGTDFTLSINSVESEDIAD |
| | | YYCQQNNNWPTTFGAGTKLELKTSDYKDDDDKTTTPAPRPPTPAPTIAS |
| | | QPLSLRPEACRPAAGGAVHTRGLDFACDIYIWAPLAGTCGVLLLSLVITL |
| | | YKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTY |
| | | DALHMQALPPR |
| SEQ ID | C1100 | MALPVTALLLPLALLLHAARPDILLTQSPVILSVSPGERVSFSCRASASIGT |
| NO: 10 | | SIHWYQQRTNGSPRLLIKAASESISGIPSRFSGSGSGTDFTLSINSVESEDIA |
| | | DYYCQQSNSAPTTFGAGTKLELKGSSGGSGGSGGSGLSGRSDNHGSSGT |
| | | QVQLKQSGPGLVQPSQSLSITCTVSGFSLTNYGVHWVRQSPGKGLEWLG |
| | | VIWSGGNTDYNTPFTSRLSINKDNSKSQVFFKMNSLQSNDTAIYYCARAL |
| | | TYYDYEFAYWGQGTLVTVSATSDYKDDDDKTTTPAPRPPTPAPTIASQP |
| | | LSLRPEACRPAAGGAVHTRGLDFACDIYIWAPLAGTCGVLLLSLVITLYK |
| | | RGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDA |
| | | LHMQALPPR |
| SEQ ID | C1333 | MALPVTALLLPLALLLHAARPDIQMTQSPSTLSASVGDRVTITCKCQLSV |
| NO: 11 | | GYMHWYQQKPGKAPKLLIYDTSKLASGVPSRFSGSGSGTEFTLTISSLQP |
| | | DDFATYYCFQGSGYPFTFGGGTKLEIKGSSGGSGGSGGSGLSGRSDNHGS |
| | | SGTQVQLQESGPGLVKPSETLSLTCTVSGGSVSSGDYYWTWIRQSPGKG |
| | | LEWIGHIYYSGNTNYNPSLKSRLTISIDTSKTQFSLKLSSVTAADTAIYYC |
| | | VRDRVTGAFDIWGQGTMVTVSSTSEQKLISEEDLTTTPAPRPPTPAPTIAS |
| | | QPLSLRPEACRPAAGGAVHTRGLDFACDIYIWAPLAGTCGVLLLSLVITL |
| | | YKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTY |
| | | DALHMQALPPR |
| SEQ ID | C1334 | MALPVTALLLPLALLLHAARPQVTLRESGPALVKPTQTLTLTCTFSGFSLS |
| NO: 12 | | TSGMSVGWIRQPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGLSGRSDNHGSSGTDIQMTQSPSSLSASVGDRVTITCQASQDIS |
| | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQQKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRQSPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | C1335 | MALPVTALLLPLALLLHAARPQVTLRESGPALVKPTQTLTLTCTFSGFSLS |
| NO: 13 | | TSGMSVGWIRQPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGLSGRSDNHGSSGTDIQMTQSPSSLSASVGDRVTITCQASQDIS |
| | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPR |
| SEQ ID | C1336 | MALPVTALLLPLALLLHAARPDIQMTQSPSSLSASVGDRVTITCQASQDIS |
| NO: 14 | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPQVQLQESGPGLVKPSETLSLTCTVSGGSVSSGDYYWTWIRQSP |
| | | GKGLEWIGHIYYSGNTNYNPSLKSRLTISIDTSKTQFSLKLSSVTAADTAI |
| | | YYCVRDRVTGAFDIWGQGTMVTVSSTSEQKLISEEDLTTTPAPRPPTPAP |
| | | TIASQPLSLRPEACRPAAGGAVHTRGLDFACDIYIWAPLAGTCGVLLLSL |
| | | VITLYKRGRKKLLYIFKQPFMRPVQTTQEEDGCSCRFPEEEEGGCELRVK |
| | | FSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATK |
| | | DTYDALHMQALPPR |
| SEQ ID | C1402 | MALPVTALLLPLALLLHAARPQVTLRESGPALVKPTQTLTLTCTFSGFSLS |
| NO: 15 | | TSGMSVGWIRQPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGGGGSGGGSGSSGTDIQMTQSPSSLSASVGDRVTITCQASQDI |
| | | SNYLNWYQQKPGKAPKLLIYDASNLETGVPSRFSGSGSGTDFTFTISSLQP |
| | | EDIATYFCQHFDHLPLAFGGGTKVEIKTSDYKDDDDKTTTPAPRPPTPAP |
| | | TIASQPLSLRPEACRPAAGGAVHTRGLDFACDIYIWAPLAGTCGVLLLSL |
| | | VITLYKRGRKKLLYIFKQPFMRPVQTTQEEDGCSCRFPEEEEGGCELRVK |
| | | FSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATK |
| | | DTYDALHMQALPPRGSGATNFSLLKQAGDVEENPGPMALPVTALLLPLA |
| | | LLLHAARPDIQMTQSPSTLSASVGDRVTITCKCQLSVGYMHWYQQKPGK |
| | | APKLLIYDTSKLASGVPSRFSGSGSGTEFTLTISSLQPDDFATYYCFQGSG |
| | | YPFTFGGGTKLEIKGSSGGSGGSGGSGGGGSGGGSGSSGTQVQLQESGPG |
| | | LVKPSETLSLTCTVSGGSVSSGDYYWTWIRQSPGKGLEWIGHIYYSGNTN |
| | | YNPSLKSRLTISIDTSKTQFSLKLSSVTAADTAIYYCVRDRVTGAFDIWGQ |
| | | GTMVTVSSASEQKLISEEDLTTTPAPRPPTPAPTIASQPLSLRPEACRPAAG |
| | | GAVHTRGLDFACDIYIWAPLAGTCGVLLLSLVITLYKRGRKKLLYIFKQP |
| | | FMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| SEQ ID | C1528 | MALPVTALLLPLALLLHAARPQVTLRESGPALVKPTQTLTLTCTFSGFSLS |
| NO: 16 | | TSGMSVGWIRQPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGLSGRSDNHGSSGTDIQMTQSPSSLSASVGDRVTITCQASQDIS |
| | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQQKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRQSPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLY |
| SEQ ID | C1529 | MALPVTALLLPLALLLHAARPDIQMTQSPSSLSASVGDRVTITCQASQDIS |
| NO: 17 | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPQVQLQESGPGLVKPSETLSLTCTVSGGSVSSGDYYWTWIRQSP |
| | | GKGLEWIGHIYYSGNTNYNPSLKSRLTISIDTSKTQFSLKLSSVTAADTAI |
| | | YYCVRDRVTGAFDIWGQGTMVTVSSTSEQKLISEEDLTTTPAPRPPTPAP |
| | | TIASQPLSLRPEACRPAAGGAVHTRGLDFACDIYIWAPLAGTCGVLLLSL |
| | | VITLY |
| SEQ ID | C1530 | MALPVTALLLPLALLLHAARPQVTLRESGPALVKPTQTLTLTCTFSGFSLS |
| NO: 18 | | TSGMSVGWIRQPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGGGGSGGGSGSSGTDIQMTQSPSSLSASVGDRVTITCQASQDI |
| | | SNYLNWYQQKPGKAPKLLIYDASNLETGVPSRFSGSGSGTDFTFTISSLQP |
| | | EDIATYFCQHFDHLPLAFGGGTKVEIKTSDYKDDDDKTTTPAPRPPTPAP |
| | | TIASQPLSLRPEACRPAAGGAVHTRGLDFACDIYIWAPLAGTCGVLLLSL |
| | | VITLYKRGRKKLLYIFKQPFMRPVQTTQEEDGCSCRFPEEEEGGCELRVK |
| | | FSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATK |
| | | DTYDALHMQALPPRGSGATNFSLLKQAGDVEENPGPMALPVTALLLPLA |
| | | LLLHAARPDIQMTQSPSTLSASVGDRVTITCKCQLSVGYMHWYQQKPGK |
| | | APKLLIYDTSKLASGVPSRFSGSGSGTEFTLTISSLQPDDFATYYCFQGSG |
| | | YPFTFGGGTKLEIKGSSGGSGGSGGSGGGGSGGGSGSSGTQVQLQESGPG |
| | | LVKPSETLSLTCTVSGGSVSSGDYYWTWIRQSPGKGLEWIGHIYYSGNTN |
| | | YNPSLKSRLTISIDTSKTQFSLKLSSVTAADTAIYYCVRDRVTGAFDIWGQ |
| | | GTMVTVSSASEQKLISEEDLTTTPAPRPPTPAPTIASQPLSLRPEACRPAAG |
| | | GAVHTRGLDFACDIYIWAPLAGTCGVLLLSLVITLY |
| SEQ ID | C1536 | MALPVTALLLPLALLLHAARPQVTLRESGPALVKPTQTLTLTCTFSGFSLS |
| NO: 19 | | TSGMSVGWIREPPGKALEWLADIWWDDKKDYNPSLKSRLTISKDTSKNQ |
| | | VVLKVTNMDPADTATYYCARSMITNWYFDVWGAGTTVTVSSGSSGGS |
| | | GGSGGSGLSGRSDNHGSSGTDIQMTQSPSSLSASVGDRVTITCQASQDIS |
| | | NYLNWYQKKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQEKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRKSPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | C1537 | MALPVTALLLPLALLLHAARPQVTLRESGPALVKPTQTLTLTCTFSGFSLS |
| NO: 20 | | TSGMSVGWIRKPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGLSGRSDNHGSSGTDIQMTQSPSSLSASVGDRVTITCQASQDIS |
| | | NYLNWYQEKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQKKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRESPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | C1631 | MALPVTALLLPLALLLHAARPQVTLRESGPALVKPTQTLTLTCTFSGFSLS |
| NO: 21 | | TSGMSVGWIREPPGKALEWLADIWWDDKKDYNPSLKSRLTISKDTSKNQ |
| | | VVLKVTNMDPADTATYYCARSMITNWYFDVWGAGTTVTVSSGSSGGS |
| | | GGSGGSGLSGRSDNHGSSGTDIQMTQSPSSLSASVGDRVTITCQASQDIS |
| | | NYLNWYQKKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQQKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRQSPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | C1740 | MALPVTALLLPLALLLHAARPQVTLRESGPALVKPTQTLTLTCTFSGFSLS |
| NO: 22 | | TSGMSVGWIRQPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGLSGRSDNHGSSGTDIQMTQSPSSLSASVGDRVTITCQASQDIS |
| | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQEKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRKSPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | C1747 | MALPVTALLLPLALLLHAARPDIQMTQSPSSLSASVGDRVTITCQASQDIS |
| NO: 23 | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQQKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRQSPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | C1748 | MALPVTALLLPLALLLHAARPDIQMTQSPSSLSASVGDRVTITCQASQDIS |
| NO: 24 | | NYLNWYQQKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQQKPGKAP |
| | | KLLIYDTSKLASGVPSRFSGSGSGTEFTLTISSLQPDDFATYYCFQGSGYPF |
| | | TFGGGTKLEIKGSSGGSGGSGGSGGGGSGGGSGSSGTQVQLQESGPGLV |
| | | KPSETLSLTCTVSGGSVSSGDYYWTWIRQSPGKGLEWIGHIYYSGNTNY |
| | | NPSLKSRLTISIDTSKTQFSLKLSSVTAADTAIYYCVRDRVTGAFDIWGQG |
| | | TMVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| SEQ ID | Protease | KRALGLPG |
| NO: 25 | Cleavage Site - | |
| | MMP7 | |
| SEQ ID | Protease | (DE)8RPLALWRS(DR)8 |
| NO: 26 | Cleavage Site - | |
| | MMP7 | |
| SEQ ID | Protease | PR(S/T)(L/I)(S/T) |
| NO: 27 | Cleavage Site - | |
| | MMP9 | |
| SEQ ID | Protease | LEATA |
| NO: 28 | Cleavage Site - | |
| | MMP9 | |
| SEQ ID | Protease | GGAANLVRGG |
| NO: 29 | Cleavage Site - | |
| | MMP11 | |
| SEQ ID | Protease | SGRIGFLRTA |
| NO: 30 | Cleavage Site - | |
| | MMP14 | |
| SEQ ID | Protease | PLGLAG |
| NO: 31 | Cleavage Site - | |
| | MMP | |
| SEQ ID | Protease | PLGLAX |
| NO: 32 | Cleavage Site - | |
| | MMP | |
| SEQ ID | Protease | PLGC(me)AG |
| NO: 33 | Cleavage Site - | |
| | MMP | |
| SEQ ID | Protease | ESPAYYTA |
| NO: 34 | Cleavage Site - | |
| | MMP | |
| SEQ ID | Protease | RLQLKL |
| NO: 35 | Cleavage Site - | |
| | MMP | |
| SEQ ID | Protease | RLQLKAC |
| NO: 36 | Cleavage Site - | |
| | MMP | |
| SEQ ID | Protease | EP(Cit)G(Hof)YL |
| NO: 37 | Cleavage Site - | |
| | MMP2, MMP9, | |
| | MMP14 | |
| SEQ ID | Protease | SGRSA |
| NO: 38 | Cleavage Site - | |
| | Urokinase | |
| | plasminogen | |
| | activator (uPA) | |
| SEQ ID | Protease | DAFK |
| NO: 39 | Cleavage Site - | |
| | Urokinase | |
| | plasminogen | |
| | activator (uPA) | |
| SEQ ID | Protease | GGGRR |
| NO: 40 | Cleavage Site - | |
| | Urokinase | |
| | plasminogen | |
| | activator (uPA) | |
| SEQ ID | Protease | GFLG |
| NO: 41 | Cleavage Site - | |
| | Lysosomal | |
| | Enzyme | |
| SEQ ID | Protease | ALAL |
| NO: 42 | Cleavage Site - | |
| | Lysosomal | |
| | Enzyme | |
| SEQ ID | Protease | FK |
| NO: 43 | Cleavage Site - | |
| | Lysosomal | |
| | Enzyme | |
| SEQ ID | Protease | NLL |
| NO: 44 | Cleavage Site - | |
| | Cathepsin B | |
| SEQ ID | Protease | PIC(Et)FF |
| NO: 45 | Cleavage Site - | |
| | Cathepsin D | |
| SEQ ID | Protease | GGPRGLPG |
| NO: 46 | Cleavage Site - | |
| | Cathepsin K | |
| SEQ ID | Protease | HSSKLQ |
| NO: 47 | Cleavage Site - | |
| | Prostate Specific | |
| | Antigen | |
| SEQ ID | Protease | HSSKLQL |
| NO: 48 | Cleavage Site - | |
| | Prostate Specific | |
| | Antigen | |
| SEQ ID | Protease | HSSKLQEDA |
| NO: 49 | Cleavage Site - | |
| | Prostate Specific | |
| | Antigen | |
| SEQ ID | Protease | LVLASSSFGY |
| NO: 50 | Cleavage Site - | |
| | Herpes Simplex | |
| | Virus Protease | |
| SEQ ID | Protease | GVSQNYPIVG |
| NO: 51 | Cleavage Site - | |
| | HIV Protease | |
| SEQ ID | Protease | GVVQASCRLA |
| NO: 52 | Cleavage Site - | |
| | CMV Protease | |
| SEQ ID | Protease | F(Pip)RS |
| NO: 53 | Cleavage Site - | |
| | Thrombin | |
| SEQ ID | Protease | DPRSFL |
| NO: 54 | Cleavage Site - | |
| | Thrombin | |
| SEQ ID | Protease | PPRSFL |
| NO: 55 | Cleavage Site - | |
| | Thrombin | |
| SEQ ID | Protease | DEVD |
| NO: 56 | Cleavage Site - | |
| | Caspase-3 | |
| SEQ ID | Protease | DEVDP |
| NO: 57 | Cleavage Site - | |
| | Caspase-3 | |
| SEQ ID | Protease | KGSGDVEG |
| NO: 58 | Cleavage Site - | |
| | Caspase-3 | |
| SEQ ID | Protease | GWEHDG |
| NO: 59 | Cleavage Site - | |
| | Interleukin 1β | |
| | converting | |
| | enzyme | |
| SEQ ID | Protease | EDDDDKA |
| NO: 60 | Cleavage Site - | |
| | Enterokinase | |
| SEQ ID | Protease | KQEQNPGST |
| NO: 61 | Cleavage Site - | |
| | FAP | |
| SEQ ID | Protease | GKAFRR |
| NO: 62 | Cleavage Site - | |
| | Kallikrein 2 | |
| SEQ ID | Protease | DAFK |
| NO: 63 | Cleavage Site - | |
| | Plasmin | |
| SEQ ID | Protease | DVLK |
| NO: 64 | Cleavage Site - | |
| | Plasmin | |
| SEQ ID | Protease | DAFK |
| NO: 65 | Cleavage Site - | |
| | Plasmin | |
| SEQ ID | Protease | ALLLALL |
| NO: 66 | Cleavage Site - | |
| | TOP | |
| SEQ ID | Exemplary linker | GGSGGS |
| NO: 67 | sequence | |
| SEQ ID | Exemplary linker | GGSG |
| NO: 68 | sequence | |
| SEQ ID | Exemplary linker | GGSGG |
| NO: 69 | sequence | |
| SEQ ID | Exemplary linker | GSGSG |
| NO: 70 | sequence | |
| SEQ ID | Exemplary linker | GSGGG |
| NO: 71 | sequence | |
| SEQ ID | Exemplary linker | GGGSG |
| NO: 72 | sequence | |
| SEQ ID | Exemplary linker | GSSSG |
| NO: 73 | sequence | |
| SEQ ID | Exemplary linker | GSSGGSGGSGGSG |
| NO: 74 | sequence | |
| SEQ ID | Exemplary linker | GSSGGSGGSGG |
| NO: 75 | sequence | |
| SEQ ID | Exemplary linker | GSSGGSGGSGGS |
| NO: 76 | sequence | |
| SEQ ID | Exemplary linker | GSSGGSGGSGGSGGGS |
| NO: 77 | sequence | |
| SEQ ID | Exemplary linker | GSSGGSGGSG |
| NO: 78 | sequence | |
| SEQ ID | Exemplary linker | GSSGGSGGSGS |
| NO: 79 | sequence | |
| SEQ ID | Exemplary linker | GSS |
| NO: 80 | sequence | |
| SEQ ID | Exemplary linker | GGS |
| NO: 81 | sequence | |
| SEQ ID | Exemplary linker | GGGS |
| NO: 82 | sequence | |
| SEQ ID | Exemplary linker | GSSGT |
| NO: 83 | sequence | |
| SEQ ID | Exemplary linker | GSSG |
| NO: 84 | sequence | |
| SEQ ID | Protease | ISSGLLSS |
| NO: 85 | cleavage site | |
| SEQ ID | Protease | QNQALRMA |
| NO: 86 | cleavage site | |
| SEQ ID | Protease | AQNLLGMV |
| NO: 87 | cleavage site | |
| SEQ ID | Protease | STFPFGMF |
| NO: 88 | cleavage site | |
| SEQ ID | Protease | PVGYTSSL |
| NO: 89 | cleavage site | |
| SEQ ID | Protease | DWLYWPGI |
| NO: 90 | cleavage site | |
| SEQ ID | Protease | MIAPVAYR |
| NO: 91 | cleavage site | |
| SEQ ID | Protease | RPSPMWAY |
| NO: 92 | cleavage site | |
| SEQ ID | Protease | WATPRPMR |
| NO: 93 | cleavage site | |
| SEQ ID | Protease | FRLLDWQW |
| NO: 94 | cleavage site | |
| SEQ ID | Protease | LKAAPRWA |
| NO: 95 | cleavage site | |
| SEQ ID | Protease | GPSHLVLT |
| NO: 96 | cleavage site | |
| SEQ ID | Protease | LPGGLSPW |
| NO: 97 | cleavage site | |
| SEQ ID | Protease | MGLFSEAG |
| NO: 98 | cleavage site | |
| SEQ ID | Protease | SPLPLRVP |
| NO: 99 | cleavage site | |
| SEQ ID | Protease | RMHLRSLG |
| NO: 100 | cleavage site | |
| SEQ ID | Protease | LAAPLGLL |
| NO: 101 | cleavage site | |
| SEQ ID | Protease | AVGLLAPP |
| NO: 102 | cleavage site | |
| SEQ ID | Protease | LLAPSHRA |
| NO: 103 | cleavage site | |
| SEQ ID | Protease | PAGLWLDP |
| NO: 104 | cleavage site | |
| SEQ ID | Protease | ISSGLSS |
| NO: 105 | cleavage site | |
| SEQ ID | Protease | ISSGL |
| NO: 106 | cleavage site | |
| SEQ ID | Protease | ISSGLLS |
| NO: 107 | cleavage site | |
| SEQ ID | Protease | ISSGLL |
| NO: 108 | cleavage site | |
| SEQ ID | Protease | VHMPLGFLGP |
| NO: 109 | cleavage site | |
| SEQ ID | human CD3- | MQSGTHWRVLGLCLLSVGVWGQDGNEEMGGITQTPYKVSISGTTVILTC |
| NO: 110 | epsilon | PQYPGSEILWQHNDKNIGGDEDDKNIGSDEDHLSLKEFSELEQSGYYVCY |
| | polypeptide | PRGSKPEDANFYLYLRARVCENCMEMDVMSVATIVIVDICITGGLLLLVY |
| | | YWSKNRKAKAKPVTRGAGAGGRQRGQNKERPPPVPNPDYEPIRKGQRD |
| | | LYSGLNQRRI |
| SEQ ID | human CD3- | MEQGKGLAVLILAIILLQGTLAQSIKGNHLVKVYDYQEDGSVLLTCDAE |
| NO: 111 | gamma | AKNITWFKDGKMIGFLTEDKKKWNLGSNAKDPRGMYQCKGSQNKSKP |
| | polypeptide | LQVYYRMCQNCIELNAATISGFLFAEIVSIFVLAVGVYFIAGQDGVRQSR |
| | | ASDKQTLLPNDQLYQPLKDREDDQYSHLQGNQLRRN |
| SEQ ID | human CD3- | MEHSTFLSGLVLATLLSQVSPFKIPIEELEDRVFVNCNTSITWVEGTVGTL |
| NO: 112 | delta polypeptide | LSDITRLDLGKRILDPRGIYRCNGTDIYKDKESTVQVHYRMCQSCVELDP |
| | | ATVAGIIVTDVIATLLLALGVFCFAGHETGRLSGAADTQALLRNDQVYQP |
| | | LRDRDDAQYSHLGGNWARNK |
| SEQ ID | human CD3-zeta | MKWKALFTAAILQAQLPITEAQSFGLLDPKLCYLLDGILFIYGVILTALFL |
| NO: 113 | polypeptide | RVKFSRSADAPAYQQGQNQLYNELNLGRREEYDVLDKRRGRDPEMGG |
| | | KPQRRKNPQEGLYNELQKDKMAEAYSEIGMKGERRRGKGHDGLYQGLS |
| | | TATKDTYDALHMQALPPR |
| SEQ ID | human TCR | MAGTWLLLLLALGCPALPTGVGGTPFPSLAPPIMLLVDGKQQMVVVCL |
| NO: 114 | alpha chain | VLDVAPPGLDSPIWFSAGNGSALDAFTYGPSPATDGTWTNLAHLSLPSEE |
| | | LASWEPLVCHTGPGAEGHSRSTQPMHLSGEASTARTCPQEPLRGTPGGA |
| | | LWLGVLRLLLFKLLLFDLLLTCSCLCDPAGPLPSPATTTRLRALGSHRLHP |
| | | ATETGGREATSSPRPQPRDRRWGDTPPGRKPGSPVWGEGSYLSSYPTCP |
| | | AQAWCSRSALRAPSSSLGAFFAGDLPPPLQAGAA |
| SEQ ID | human TCR | PNIQNPDPAVYQLRDSKSSDKSVCLFTDFDSQTNVSQSKDSDVYITDKTV |
| NO: 115 | alpha chain C | LDMRSMDFKSNSAVAWSNKSDFACANAFNNSIIPEDTFFPSPESSCDVKL |
| | region | VEKSFETDTNLNFQNLSVIGFRILLLKVAGFNLLMTLRLWSS |
| SEQ ID | human TCR | MAMLLGASVLILWLQPDWVNSQQKNDDQQVKQNSPSLSVQEGRISILNC |
| NO: 116 | alpha chain V | DYTNSMFDYFLWYKKYPAEGPTFLISISSIKDKNEDGRFTVFLNKSAKHL |
| | region CTL-L17 | SLHIVPSQPGDSAVYFCAAKGAGTASKLTFGTGTRLQVTL |
| SEQ ID | human TCR beta | EDLNKVFPPEVAVFEPSEAEISHTQKATLVCLATGFFPDHVELSWWVNG |
| NO: 117 | chain C region | KEVHSGVSTDPQPLKEQPALNDSRYCLSSRLRVSATFWQNPRNHFRCQV |
| | | QFYGLSENDEWTQDRAKPVTQIVSAEAWGRADCGFTSVSYQQGVLSATI |
| | | LYEILLGKATLYAVLVSALVLMAMVKRKDF |
| SEQ ID | human TCR beta | MGTSLLCWMALCLLGADHADTGVSQNPRHNITKRGQNVTFRCDPISEH |
| NO: 118 | chain V region | NRLYWYRQTLGQGPEFLTYFQNEAQLEKSRLLSDRFSAERPKGSFSTLEI |
| | | QRTEQGDSAMYLCASSLAGLNQPQHFGDGTRLSIL |
| SEQ ID | TCR beta chain | MDSWTFCCVSLCILVAKHTDAGVIQSPRHEVTEMGQEVTLRCKPISGHN |
| NO: 119 | V region YT35 | SLFWYRQTMMRGLELLIYFNNNVPIDDSGMPEDRFSAKMPNASFSTLKI |
| | | QPSEPRDSAVYFCASSFSTCSANYGYTFGSGTRLTVV |
| SEQ ID | Exemplary linker | GGGGSGGGGS |
| NO: 120 | sequence | |
| SEQ ID | Exemplary linker | GGTGGCGGAGGTTCTGGAGGTGGAGGTTCC |
| NO: 121 | sequence | |
| SEQ ID | Exemplary linker | GG |
| NO: 122 | sequence | |
| SEQ ID | CD8a signal | MALPVTALLLPLALLLHAARP |
| NO: 123 | Peptide | |
| SEQ ID | cetuximab Vh | QVQLKQSGPGLVQPSQSLSITCTVSGFSLTNYGVHWVRQSPGKGLEWLG |
| NO: 124 | | VIWSGGNTDYNTPFTSRLSINKDNSKSQVFFKMNSLQSNDTAIYYCARAL |
| | | TYYDYEFAYWGQGTLVTVSA |
| SEQ ID | cetuximab Vl | DILLTQSPVILSVSPGERVSFSCRASQSIGTNIHWYQQRTNGSPRLLIKYAS |
| NO: 125 | | ESISGIPSRFSGSGSGTDFTLSINSVESEDIADYYCQQNNNWPTTFGAGTKL |
| | | ELK |
| SEQ ID | inactivated | QVQLKQSGPGLVQPSQSLSITCTVSGFSLTSYGVHWVRQSPGKGLEWLG |
| NO: 126 | cetuximab Vh | VIASGGSTDYNTPFTSRLSINKDNSKSQVFFKMNSLQSNDTAIYYCARAL |
| | | TAARAEFAYWGQGTLVTVSA |
| SEQ ID | inactivated | DILLTQSPVILSVSPGERVSFSCRASASIGTSIHWYQQRTNGSPRLLIKAAS |
| NO: 127 | cetuximab Vl | ESISGIPSRFSGSGSGTDFTLSINSVESEDIADYYCQQSNSAPTTFGAGTKL |
| | | ELK |
| SEQ ID | panitumamab Vh | QVQLQESGPGLVKPSETLSLTCTVSGGSVSSGDYYWTWIRQSPGKGLEW |
| NO: 128 | | IGHIYYSGNTNYNPSLKSRLTISIDTSKTQFSLKLSSVTAADTAIYYCVRDR |
| | | VTGAFDIWGQGTMVTVSS |
| SEQ ID | panitumamab Vl | DIQMTQSPSSLSASVGDRVTITCQASQDISNYLNWYQQKPGKAPKLLIYD |
| NO: 129 | | ASNLETGVPSRFSGSGSGTDFTFTISSLQPEDIATYFCQHFDHLPLAFGGG |
| | | TKVEIK |
| SEQ ID | panitumamab Vh | QVTLRESGPALVKPTQTLTLTCTFSGFSLSTSGMSVGWIRQPPGKALEWL |
| NO: 130 | | ADIWWDDKKDYNPSLKSRLTISKDTSKNQVVLKVTNMDPADTATYYCA |
| | | RSMITNWYFDVWGAGTTVTVSS |
| SEQ ID | palivizumab Vl | DIQMTQSPSTLSASVGDRVTITCKCQLSVGYMHWYQQKPGKAPKLLIYD |
| NO: 131 | | TSKLASGVPSRFSGSGSGTEFTLTISSLQPDDFATYYCFQGSGYPFTFGGG |
| | | TKLEIK |
| SEQ ID | palivizumab Vh | QVTLRESGPALVKPTQTLTLTCTFSGFSLSTSGMSVGWIREPPGKALEWL |
| NO: 132 | Q39E | ADIWWDDKKDYNPSLKSRLTISKDTSKNQVVLKVTNMDPADTATYYCA |
| | | RSMITNWYFDVWGAGTTVTVSS |
| SEQ ID | panitumamab Vl | DIQMTQSPSSLSASVGDRVTITCQASQDISNYLNWYQKKPGKAPKLLIYD |
| NO: 133 | Q38K | ASNLETGVPSRFSGSGSGTDFTFTISSLQPEDIATYFCQHFDHLPLAFGGG |
| | | TKVEIK |
| SEQ ID | palivizumab Vl | DIQMTQSPSTLSASVGDRVTITCKCQLSVGYMHWYQEKPGKAPKLLIYD |
| NO: 134 | Q38E | TSKLASGVPSRFSGSGSGTEFTLTISSLQPDDFATYYCFQGSGYPFTFGGG |
| | | TKLEIK |
| SEQ ID | panitumamab Vh | QVQLQESGPGLVKPSETLSLTCTVSGGSVSSGDYYWTWIRKSPGKGLEW |
| NO: 135 | Q39K | IGHIYYSGNTNYNPSLKSRLTISIDTSKTQFSLKLSSVTAADTAIYYCVRDR |
| | | VTGAFDIWGQGTMVTVSS |
| SEQ ID | palivizumab Vh | QVTLRESGPALVKPTQTLTLTCTFSGFSLSTSGMSVGWIRKPPGKALEWL |
| NO: 136 | Q39K | ADIWWDDKKDYNPSLKSRLTISKDTSKNQVVLKVTNMDPADTATYYCA |
| | | RSMITNWYFDVWGAGTTVTVSS |
| SEQ ID | panitumamab Vl | DIQMTQSPSSLSASVGDRVTITCQASQDISNYLNWYQEKPGKAPKLLIYD |
| NO: 137 | Q38E | ASNLETGVPSRFSGSGSGTDFTFTISSLQPEDIATYFCQHFDHLPLAFGGG |
| | | TKVEIK |
| SEQ ID | palivizumab Vl | DIQMTQSPSTLSASVGDRVTITCKCQLSVGYMHWYQKKPGKAPKLLIYD |
| NO: 138 | Q38K | TSKLASGVPSRFSGSGSGTEFTLTISSLQPDDFATYYCFQGSGYPFTFGGG |
| | | TKLEIK |
| SEQ ID | panitumamab Vh | QVQLQESGPGLVKPSETLSLTCTVSGGSVSSGDYYWTWIRESPGKGLEWI |
| NO: 139 | Q39E | GHIYYSGNTNYNPSLKSRLTISIDTSKTQFSLKLSSVTAADTAIYYCVRDR |
| | | VTGAFDIWGQGTMVTVSS |
| SEQ ID | CD8a hinge / | TTTPAPRPPTPAPTIASQPLSLRPEACRPAAGGAVHTRGLDFACDIYIWAP |
| NO: 140 | transmembrane | LAGTCGVLLLSLVITLY |
| | sequence | |
| SEQ ID | 4-1BB | KRGRKKLLYIFKQPFMRPVQTTQEEDGCSCRFPEEEEGGCEL |
| NO: 141 | intracellular | |
| | domain | |
| SEQ ID | CD3z | RVKFSRSADAPAYQQGQNQLYNELNLGRREEYDVLDKRRGRDPEMGG |
| NO: 142 | intracellular | KPRRKNPQEGLYNELQKDKMAEAYSEIGMKGERRRGKGHDGLYQGLST |
| | domain | ATKDTYDALHMQALPPR |
| SEQ ID | protease | GSSGGSGGSGGSGLSGRSDNHGSSGT |
| NO: 143 | cleavable linker | |
| SEQ ID | P2A exon | GSGATNFSLLKQAGDVEENPGP |
| NO: 144 | skipping peptide | |
| SEQ ID | C1634 | MALPVTALLLPLALLLHAARPQVTLRESGPALVKPTQTLTLTCTFSGFSLS |
| NO: 145 | | TSGMSVGWIRKPPGKALEWLADIWWDDKKDYNPSLKSRLTISKDTSKN |
| | | QVVLKVTNMDPADTATYYCARSMITNWYFDVWGAGTTVTVSSGSSGG |
| | | SGGSGGSGLSGRSDNHGSSGTDIQMTQSPSSLSASVGDRVTITCQASQDIS |
| | | NYLNWYQEKPGKAPKLLIYDASNLETGVPSRFSGSGSGTDFTFTISSLQPE |
| | | DIATYFCQHFDHLPLAFGGGTKVEIKTSDYKDDDDKTTTPAPRPPTPAPTI |
| | | ASQPLSLRPEACRPAAGGAVHTRGLDFACDIYIWAPLAGTCGVLLLSLVI |
| | | TLYKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPRGSGATNFSLLKQAGDVEENPGPMALPVTALLLPLALL |
| | | LHAARPDIQMTQSPSTLSASVGDRVTITCKCQLSVGYMHWYQQKPGKAP |
| | | KLLIYDTSKLASGVPSRFSGSGSGTEFTLTISSLQPDDFATYYCFQGSGYPF |
| | | TFGGGTKLEIKGSSGGSGGSGGSGLSGRSDNHGSSGTQVQLQESGPGLVK |
| | | PSETLSLTCTVSGGSVSSGDYYWTWIRQSPGKGLEWIGHIYYSGNTNYNP |
| | | SLKSRLTISIDTSKTQFSLKLSSVTAADTAIYYCVRDRVTGAFDIWGQGT |
| | | MVTVSSASEQKLISEEDLTTTPAPRPPTPAPTIASQPLSLRPEACRPAAGG |
| | | AVHTRGLDFACDIYIWAPLAGTCGVLLLSLVITLYKRGRKKLLYIFKQPF |
| | | MRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAE |
| | | AYSEIGMKGERRRGKGHDGLYQGLSTATKDTYDALHMQALPPR |

What is claimed is:

1. An activatable receptor comprising:
(a) an antigen binding domain comprising
(i) a VH target-binding domain (VH) and an inactive VL domain (iVL) that binds to the VH domain, wherein the VH and iVL are attached via a linker (L1); and
(ii) a VL target-binding domain (VL) and an inactive VH domain (iVH) that binds to the VL domain, wherein the VL and iVH are attached via a linker (L2);
(b) a transmembrane domain; and
(c) an intracellular signaling domain;
wherein L1 comprises a first protease cleavage site and L2 comprises a second protease cleavage site.

2. An activatable receptor comprising:
(a) an antigen binding domain comprising
(i) a VH target-binding domain (VH) and an inactive VL domain (iVL) that binds to the VH domain, wherein the VH and iVL are attached via a linker (L1); and
(ii) a VL target-binding domain (VL) and an inactive VH domain (iVH) that binds to the VL domain, wherein the VL and iVH are attached via a linker (L2);
(b) a transmembrane domain; and
(c) an intracellular signaling domain;
wherein iVL comprises a first protease cleavage site and iVH comprises a second protease cleavage site.

3. The activatable receptor of claim 2, wherein the first protease cleavage site and the second protease cleavage site are located within a CDR1, CDR2, or CDR3.

4. The activatable receptor of any of claims 1-3, wherein the transmembrane domain comprises a transmembrane domain of a T-cell receptor, CD28, CD3, CD45, CD4, CD5, CD8, CD9, CD16, CD22, CD33, CD37, CD64, CD80, CD86, CD134, CD137, or CD154, or a portion thereof.

5. The activatable receptor of any of claims 1-4, wherein the intracellular signaling domain comprises an intracellular domain of CD3 zeta, FcR gamma, FcR beta, CD3 gamma, CD3 delta, CD3 epsilon, CD5, CD22, CD79a, CD79b, or CD66d, or a portion thereof.

6. The activatable receptor of any of claims 1-5, wherein the intracellular signaling domain comprises a costimulatory domain.

7. The activatable receptor of claim 6, wherein the costimulatory domain comprises an intracellular domain of CD27, CD28, 4-1BB, OX40, CD30, CD40, PD1, ICOS, LFA-1, CD2, CD7, LIGHT, NKG2C, B7-H3, GITR, BAFFR, HVEM, SLAMF7, NKp80, or CD160, or a portion thereof.

8. An activatable receptor comprising:
(a) an antigen binding domain comprising
(i) a VH target-binding domain (VH) and an inactive VL domain (iVL) that binds to the VH domain, wherein the VH and iVL are attached via a linker (L1); and
(ii) a VL target-binding domain (VL) and an inactive VH domain (iVH) that binds to the VL domain, wherein the VL and iVH are attached via a linker (L2); and
(b) a T Cell Receptor subunit or portion thereof;
wherein L1 comprises a first protease cleavage site and L2 comprises a second protease cleavage site.

9. An activatable receptor comprising:
(a) an antigen binding domain comprising
(i) a VH target-binding domain (VH) and an inactive VL domain (iVL) that binds to the VH domain, wherein the VH and iVL are attached via a linker (L1); and
(ii) a VL target-binding domain (VL) and an inactive VH domain (iVH) that binds to the VL domain, wherein the VL and iVH are attached via a linker (L2); and
(b) a T Cell Receptor subunit or portion thereof;
wherein iVL comprises a first protease cleavage site and iVH comprises a second protease cleavage site.

10. The activatable receptor of any of claims 1-9, wherein the T Cell Receptor subunit or portion thereof comprises a subunit from TCR-alpha, TCR-beta, CD3-gamma, CD3-epsilon, or CD3-delta, or a portion thereof.

11. An activatable receptor comprising:
(a) a first polypeptide comprising a VH target-binding domain (VH) and an inactive VL domain (iVL) that binds to the VH domain, wherein the VH and VL are attached via a linker (L1);
(b) a second polypeptide comprising a VL target-binding domain (VL) and an inactive VH domain (iVH) that binds to the VL domain, wherein the VL and iVH are attached via a linker (L2); and
(c) a transmembrane domain; and
(d) an intracellular signaling domain;
wherein L1 comprises a first protease cleavage site and L2 comprises a second protease cleavage site.

12. An activatable receptor comprising:
(a) a first polypeptide comprising a VH target-binding domain (VH) and an inactive VL domain (iVL) that binds to the VH domain, wherein the VH and iVL are attached via a linker (L1);
(b) a second polypeptide comprising a VL target-binding domain (VL) and an inactive VH domain (iVH) that binds to the VL domain, wherein the VL and iVH are attached via a linker (L2); and
(c) a transmembrane domain; and
(d) an intracellular signaling domain;
wherein iVL comprises a first protease cleavage site and iVH comprises a second protease cleavage site.

13. An activatable receptor comprising:
(a) a first polypeptide comprising (i) a VH target-binding domain (VH), (ii) an inactive VL domain (iVL) that binds to the VH domain, wherein the VH and iVL are attached via a linker (L1), and (iii) a transmembrane domain; and
(b) a second polypeptide comprising (i) a VL target-binding domain (VL), (ii) an inactive VH domain (iVH) that binds to the VL domain, wherein the VL and iVH are attached via a linker (L2), and (iii) a transmembrane domain;
wherein the first polypeptide or the second polypeptide comprises an intracellular signaling domain; and
wherein L1 comprises a first protease cleavage site and L2 comprises a second protease cleavage site.

14. An activatable receptor comprising:
(a) a first polypeptide comprising (i) a VH target-binding domain (VH), (ii) an inactive VL domain (iVL) that binds to the VH domain, wherein the VH and iVL are attached via a linker (L1), and (iii) a transmembrane domain; and
(b) a second polypeptide comprising (i) a VL target-binding domain (VL), (ii) an inactive VH domain (iVH) that binds to the VL domain, wherein the VL and iVH are attached via a linker (L2), and (iii) a transmembrane domain;
wherein the first polypeptide or the second polypeptide comprises an intracellular signaling domain; and
wherein iVL comprises a first protease cleavage site and iVH comprises a second protease cleavage site.

15. An activatable receptor comprising:
(a) a first polypeptide comprising
(i) a VH target-binding domain (VH) and an inactive VL domain (iVL) that binds to the VH domain, wherein the VH and iVL are attached via a linker (L1); and
(ii) a T Cell Receptor subunit or portion thereof; and
(b) a second polypeptide comprising
(i) a VL target-binding domain (VL) and an inactive VH domain (iVH) that binds to the VL domain, wherein the VL and iVH are attached via a linker (L2); and
(ii) a T Cell Receptor subunit or portion thereof;
wherein L1 comprises a first protease cleavage site and L2 comprises a second protease cleavage site.

16. An activatable receptor comprising:
(a) a first polypeptide comprising
(i) a VH target-binding domain (VH) and an inactive VL domain (iVL) that binds to the VH domain, wherein the VH and iVL are attached via a linker (L1); and
(ii) a T Cell Receptor subunit or portion thereof; and
(b) a second polypeptide comprising
(i) a VL target-binding domain (VL) and an inactive VH domain (iVH) that binds to the VL domain, wherein the VL and iVH are attached via a linker (L2); and
(ii) a T Cell Receptor subunit or portion thereof;
wherein iVL comprises a first protease cleavage site and iVH comprises a second protease cleavage site.

17. The activatable receptor of any of claims 11-16, wherein the first polypeptide comprises a dimerization domain.

18. The activatable receptor of any of claims 11-17, wherein the second polypeptide comprises a dimerization domain.

19. The activatable receptor of any of claims 1-18, wherein the first protease cleavage site and the second protease cleavage site are capable of being cleaved by the same protease.

20. The activatable receptor of any of claims 1-19, wherein the first protease cleavage site and the second protease cleavage site are capable of being cleaved by different proteases.

21. The activatable receptor of any of claims 1-20, wherein the first protease cleavage site and the second protease cleavage site have the same sequence.

22. The activatable receptor of any of claims 1-21, wherein the first protease cleavage site and the second protease cleavage site have different sequences.

23. The activatable receptor of any of claims 1-22, wherein the first protease cleavage site and the second protease cleavage site are capable of being cleaved by at least one of a serine protease, a cysteine protease, an aspartate protease, a threonine protease, a glutamic acid protease, a metalloproteinase, a gelatinase, and a asparagine peptide lyase.

24. The activatable receptor of any of claims 1-23, wherein the first protease cleavage site and the second protease cleavage site are capable of being cleaved at the site of a tumor.

25. The activatable receptor of any of claims 1-24, wherein VH, iVL, and L1 form a scFv.

26. The activatable receptor of any of claims 1-25, wherein VL, iVH, and L2 form a scFv.

27. The activatable receptor of any of claims 1-26, wherein iVH and iVL have a reduced binding affinity for an antigen.

28. The activatable receptor of any of claims 1-27, wherein VH and iVL are associated via a salt bridge.

29. The activatable receptor of any of claims 1-28, wherein VL and iVH are associated via a salt bridge.

30. An activatable receptor comprising:
(a) an antigen binding domain comprising
(i) a VH target-binding domain (VH);
(ii) a VL target-binding domain (VL); and
(iii) an inactive binding domain attached to VH or VL via a linker (L1);
(b) a transmembrane domain; and
(c) an intracellular signaling domain;
wherein L1 comprises a protease cleavage site.

31. An activatable receptor comprising:
(a) an antigen binding domain comprising
(i) a VH target-binding domain (VH);
(ii) a VL target-binding domain (VL); and
(iii) an inactive binding domain attached to VH or VL via a linker (L1);
(b) a transmembrane domain; and
(c) an intracellular signaling domain.
wherein the inactive binding domain comprises a protease cleavage site.

32. The activatable receptor of claim 31, wherein the protease cleavage site is located within a CDR1, CDR2, or CDR3.

33. An activatable receptor comprising:
(a) an antigen binding domain comprising
(i) a VH target-binding domain (VH);
(ii) a VL target-binding domain (VL); and
(iii) an inactive binding domain attached to VH or VL via a linker (L1); and
(b) a T Cell Receptor subunit or portion thereof;
wherein L1 comprises a protease cleavage site.

34. An activatable receptor comprising:
(a) an antigen binding domain comprising
(i) a VH target-binding domain (VH);
(ii) a VL target-binding domain (VL); and
(iii) an inactive binding domain attached to VH or VL via a linker (L1); and
(b) a T Cell Receptor subunit or portion thereof;
wherein the inactive binding domain comprises a protease cleavage site.

35. The activatable receptor of any of claims 1-34, wherein, upon activation, VH and VL associate to form an active target-binding domain.

36. An activatable receptor comprising:
(a) an antigen binding domain;
(b) an inhibitory domain that prevents binding of the target antigen binding domain to a target;
(c) a transmembrane domain; and
(d) an intracellular signaling domain
wherein the antigen binding domain and the inhibitory domain are attached via a linker comprising a protease cleavage site.

37. An activatable receptor comprising:
(a) an antigen binding domain;
(b) an inhibitory domain that prevents binding of the target antigen binding domain to a target; and
(c) a T Cell Receptor subunit or portion thereof;
wherein the antigen binding domain and the inhibitory domain are attached via a linker comprising a protease cleavage site.

38. The activatable receptor of claim 36 or 37, wherein the inhibitory domain is a sdAb, an inactive VHH domain, a peptide, or a ligand.

39. The activatable receptor of any of claims 30-38, wherein the protease cleavage site is capable of being cleaved by at least one of a serine protease, a cysteine protease, an aspartate protease, a threonine protease, a glutamic acid protease, a metalloproteinase, a gelatinase, and a asparagine peptide lyase.

40. The activatable receptor of any of claims 30-39, wherein the protease cleavage site is capable of being cleaved at the site of a tumor.

41. The activatable receptor of any of claims 1-40, wherein, upon activation, the activatable receptor binds to a tumor antigen.

42. The activatable receptor of claim 41, wherein the tumor antigen is CD 19, CD 123, CD22, CD30, CD 171, CS-1, CLL-1 (CLECL1), CD33, CD161, CD71, EGFRvDI, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC 1, EGFR, NCAM, Prosiase, PAP, ELF2M, Ephrin B2, 1GF-1 receptor, CA1X, LMP2, g 100, bcr-abl, tyrosinase, EphA2, Fucosyl GM1, sLe, GM3, TGS5, HMWMAA, o-acetyl-GD2, Folate receptor beta, TEM1/CD248, TEM7R, CLD 6, TSHR, GPRC5D, CXORF61, CD97, CD 179a, ALK, Poiysialic acid, PLAC 1, GloboH, NY-BR-1, UPK2, HAVCR1. ADRB3, PANX3, GPR20, LY6K, OR51 E2, TARP, WT1, NY-ESO-1, LAGE-1a, legumain, HPV E6, E7, MAGE-A1, ETV6-AML, sperm protein 17, XAGE1, Tie 2, MAD-CT-1, MAD-CT-2, Fos-related antigen 1, p53, p53 mutant, prostein, survivin and telornerase, PCTA-1/Galectin 8, MelanA/MART1, Ras mutant, hTERT, sarcoma translocation breakpoints, ML-IAP, ERG (TMPRSS2 ETS fusion gene), NA 17, PAX3, Androgen receptor, Cyclin B 1, MYCN, RhoC, TRP-2, CYP 1 B 1, BORIS, SART3, PAX5, OY-TES 1, LCK, AKAP-4, SSX2, RAGE-1, human telornerase reverse transcriptase, RU1, RU2, intestinal carboxyl esterase, mut hsp70-2, CD79a, CD79b, CD72, LAIR1, FCAR, LILRA2, CD300LF, CLEC 12A, BST2, EMR2, LY75, GPC3, FCRL5, AXL, IGF-1R, CD25, CD49C, gpA33, MUC-16, or IGLL1.

43. The activatable receptor of any of claims 1-42, wherein, upon activation, the activatable receptor binds to a tumor-supporting antigen.

44. The activatable receptor of claim 43, wherein the tumor-supporting antigen is a stromal cell antigen.

45. The activatable receptor of claim 44, wherein the stromal cell antigen is bone marrow stromal cell antigen 2 (BST2), fibroblast activation protein (FAP) or tenascin.

46. The activatable receptor of claim 43, wherein the tumor-supporting antigen is a myeloid-derived suppressor cell (MDSC) antigen.

47. The activatable receptor of claim 46, wherein the MDSC antigen is CD33, CD11b, CD14, CD 15, or CD66b.

48. The activatable receptor of any of claims 1-47, wherein the activatable receptor further comprises a T-cell receptor binding domain, a CD3 binding domain, a CD4 binding domain, or a CD8 binding domain.

49. An engineered cell comprising the activatable receptor of any of claims 1-48.

50. A pharmaceutical composition comprising the activatable receptor of any of claims 1-49.

51. An engineered immune cell comprising an activatable cell surface receptor polypeptide comprising:
(i) an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain, wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
(ii) a transmembrane domain; and
(iii) an intracellular signaling domain.

52. An engineered immune cell comprising an activatable cell surface receptor polypeptide comprising:
(i) an extracellular antigen-recognition polypeptide, wherein the extracellular antigen-recognition polypeptide comprises at least a binding pair, and a V_{H} domain, wherein:
a. the binding pair comprises a VL domain, a first linker domain, and a stabilizing domain, wherein the first linker domain is covalently linked to the VL domain and the stabilizing domain, wherein the VL domain and the stabilizing domain are non-covalently associated, and wherein the first linker domain or the stabilizing domain comprises a first protease cleavage site, and
wherein the stabilizing domain and the VH domain may be covalently associated by a second linker domain comprising a second protease cleavage site;
(ii) a transmembrane domain; and
(iii) an intracellular signaling domain.

53. An engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain, wherein the first linker domain is covalently linked to the first V_{L} domain and the first stabilizing domain, wherein the first V_{L} domain and the first stabilizing domain are non-covalently associated, and wherein the first linker domain and/or the first stabilizing domain include a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
wherein the extracellular antigen-recognition polypeptide is covalently connected to a T Cell Receptor subunit binding domain or a CD4 binding domain or a CD8 binding domain, wherein binding of the T Cell Receptor subunit binding domain or a CD4 binding domain or a CD8 binding domain does not substantially induce anergy of the engineered immune cell;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

54. An engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain and/or the first stabilizing domain include a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain and/or the second stabilizing domain include a second protease cleavage site;
wherein the extracellular antigen-recognition polypeptide is covalently connected to a T Cell Receptor subunit binding domain or a CD4 binding domain or a CD8 binding domain, wherein binding of the T Cell Receptor subunit binding domain or a CD4 binding domain or a CD8 binding domain does not substantially activate a T Cell Receptor present on the engineered immune cell;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

55. An engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
wherein the extracellular antigen-recognition polypeptide is covalently connected to a T Cell Polypeptide binding domain;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

56. An engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
wherein the extracellular antigen-recognition polypeptide is covalently connected to a T Cell Receptor-Associated Polypeptide binding domain;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

57. An engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain include a second protease cleavage site;
wherein the extracellular antigen-recognition polypeptide is covalently connected to a first dimerization domain; and
wherein the extracellular antigen-recognition polypeptide is covalently connected to a second dimerization domain, wherein the second dimerization domain is covalently connected to a T Cell Receptor subunit or a portion thereof selected from the group consisting of TCR-alpha, TCR-beta, CD3-gamma, CD3-epsilon, and CD3-delta or CD4 or CD8.

58. An engineered immune cell comprising an activatable cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a V_{H} domain, a first linker domain, and a first stabilizing domain wherein the first linker domain is covalently linked to the first V_{H} domain and the first stabilizing domain, wherein the first V_{H} domain and the first stabilizing domain are non-covalently associated, and wherein the first linker domain or the first stabilizing domain includes a first protease cleavage site, and
b. the second binding pair comprising a V_{L} domain, a second linker domain, and a second stabilizing domain wherein the second linker domain is covalently linked to the V_{L} domain and the second stabilizing domain, wherein the V_{L} domain and the second stabilizing domain are non-covalently associated, and wherein the second linker domain or the second stabilizing domain includes a second protease cleavage site;
wherein the extracellular antigen-recognition polypeptide is covalently connected to a first dimerization domain; and
wherein the extracellular antigen-recognition polypeptide is covalently connected to a second dimerization domain, wherein the second dimerization domain is covalently connected to a T Cell Receptor subunit or a portion thereof selected from the group consisting of TCR-alpha, TCR-beta, CD3-gamma, CD3-epsilon, and CD3-delta or CD4 or CD8.

59. An engineered immune cell comprising an activatable cell surface receptor polypeptide comprising:
(i) an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain include a second protease cleavage site;
(ii) a transmembrane domain; and
(iii) an intracellular signaling domain.

60. An engineered immune cell comprising an activatable cell surface receptor polypeptide comprising:
an extracellular antigen-recognition polypeptide, wherein the extracellular antigen-recognition polypeptide comprises at least a binding pair, and a V_{L} domain, wherein:
a. the binding pair comprises a V_{H} domain, a first linker domain, and an stabilizing domain, wherein the first linker domain is covalently linked to the V_{H} domain and the stabilizing domain, wherein the V_{H} domain and the stabilizing domain are non-covalently associated, and wherein the first linker domain or the stabilizing domain comprises a first protease cleavage site, and
wherein the stabilizing domain and the V_{L} domain may be covalently associated by a second linker domain comprising a second protease cleavage site;
(ii) a transmembrane domain; and
(iii) an intracellular signaling domain.

61. An engineered immune cell comprising a cell surface receptor polypeptide comprising:
(i) an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first stabilizing domain wherein the first linker domain is covalently linked to the first V_{L} domain and the first stabilizing domain, wherein the first V_{L} domain and the first stabilizing domain are non-covalently associated, and wherein the first linker domain or the first stabilizing domain includes a first protease cleavage site, and
b. the second binding pair comprising a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
(ii) wherein the extracellular antigen-recognition polypeptide is covalently connected to a T Cell antigen binding domain, wherein the T Cell antigen binding domain is capable of binding substantially specific to T cells; and
(iii) wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

62. An engineered immune cell comprising a cell surface receptor polypeptide comprising:
(i) an extracellular antigen-recognition polypeptide, wherein the extracellular antigen-recognition polypeptide comprises at least a first binding pair and a second binding pair, wherein:
a. the first binding pair comprises a first V_{L} domain, a first linker domain comprising a first protease cleavage site, and an inactive first V_{H} domain wherein the first linker domain is covalently linked to the first V_{L} domain and the inactive first V_{H} domain, wherein the first V_{L} domain and the inactive first V_{H} domain are non-covalently associated, and
b. the second binding pair comprising a second V_{H} domain, a second linker domain comprising a second protease cleavage site, and an inactive second V_{L} domain wherein the second linker domain is covalently linked to the second V_{H} domain and the inactive second V_{L} domain, wherein the second V_{H} domain and the inactive second V_{L} domain are non-covalently associated,
wherein the inactive first V_{H} domain and the inactive second V_{L} domain independently have a reduced binding affinity for an antigen;
wherein the inactive first V_{H} domain and the inactive second V_{L} domain are covalently associated by a third linker domain comprising a third protease cleavage site;
(ii) a transmembrane domain; and
(iii) an intracellular signaling domain.

63. An engineered immune cell comprising a cell surface receptor polypeptide comprising:
an extracellular antigen-recognition polypeptide, wherein the extracellular antigen-recognition polypeptide comprises at least a first binding pair and a second binding pair, wherein:
a. the first binding pair comprises a first V_{L} domain, a first linker domain comprising a first protease cleavage site, and an inactive first V_{H} domain wherein the first linker domain is covalently linked to the first V_{L} domain and the inactive first V_{H} domain, wherein the first V_{L} domain and the inactive first V_{H} domain are non-covalently associated, and
b. the second binding pair comprising a second V_{H} domain, a second linker domain comprising a second protease cleavage site, and an inactive second V_{L} domain wherein the second linker domain is covalently linked to the second V_{H} domain and the inactive second V_{L} domain, wherein the second V_{H} domain and the inactive second V_{L} domain are non-covalently associated,
wherein the inactive first V_{H} domain and the inactive second V_{L} domain independently have a reduced binding affinity for an antigen;
wherein the inactive first V_{H} domain and the inactive second V_{L} domain are covalently associated by a third linker domain comprising a third protease cleavage site;
wherein the extracellular antigen-recognition polypeptide is covalently connected to a T Cell Receptor subunit or portion thereof selected from the group consisting of TCR-alpha, TCR-beta, CD3-gamma, CD3-epsilon, and CD3-delta or CD4 or CD8.

64. An engineered immune cell comprising a population of cell surface receptor polypeptides comprising:
(i) a first cell surface receptor polypeptide comprising:
a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
b. a first dimerization domain;
c. a first transmembrane domain;
d. a first intracellular signaling domain; and
(ii) a second cell surface receptor polypeptide comprising:
a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{L} domain and the second stabilizing domain, wherein the V_{L} domain and the second stabilizing domain are non-covalently associated, and wherein the second linker domain or the second stabilizing domain includes a second protease cleavage site;
b. a second dimerization domain which is optionally identical to the first dimerization domain;
c. a second transmembrane domain; and
d. a second intracellular signaling domain.

65. An engineered immune cell comprising a population of cell surface receptor polypeptides comprising:
(i) a first cell surface receptor polypeptide comprising:
a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
b. a first transmembrane domain; and
c. a first intracellular signaling domain; and
(ii) a second cell surface receptor polypeptide comprising:
a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{L} domain and the second stabilizing domain, wherein the V_{L} domain and the second stabilizing domain are non-covalently associated, and wherein the second linker domain or the second stabilizing domain includes a second protease cleavage site;
b. a second transmembrane domain; and
c. a second intracellular signaling domain.

66. An engineered immune cell comprising a population of cell surface receptor polypeptides comprising:
(i) a first cell surface receptor polypeptide comprising:
a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
b. a first dimerization domain;
c. a T Cell Receptor subunit or portion thereof selected from the group consisting of TCR-alpha, TCR-beta, CD3-gamma, CD3-epsilon, and CD3-delta or CD4 or CD8; and,
(ii) a second cell surface receptor polypeptide comprising:
a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain include a second protease cleavage site;
b. a second dimerization domain which is optionally identical to the first dimerization domain;
c. a T Cell Receptor subunit or portion thereof selected from the group consisting of TCR-alpha, TCR-beta, CD3-gamma, CD3-epsilon, and CD3-delta, or CD4 or CD8.

67. An engineered immune cell comprising a population of cell surface receptor polypeptides comprising:
(i) a first cell surface receptor polypeptide comprising:
a. a first extracellular antigen-recognition polypeptide, wherein the first extracellular antigen-recognition polypeptide comprises at least a first binding pair, wherein the first binding pair comprises a V_{L} domain, a first linker domain, and a first stabilizing domain wherein the first linker domain is covalently linked to the V_{L} domain and the first stabilizing domain, wherein the V_{L} domain and the first stabilizing domain are non-covalently associated, and wherein the first linker domain or the first stabilizing domain includes a first protease cleavage site;
b. a T Cell Receptor subunit or portion thereof selected from the group consisting of TCR-alpha, TCR-beta, CD3-gamma, CD3-epsilon, and CD3-delta or CD4 or CD8; and,
(ii) a second cell surface receptor polypeptide comprising:
a. a second extracellular antigen-recognition polypeptide, wherein the second extracellular antigen-recognition polypeptide comprises at least a second binding pair wherein: the second pair comprises a V_{H} domain, a second linker domain, and a second stabilizing domain wherein the second linker domain is covalently linked to the V_{H} domain and the second stabilizing domain, wherein the V_{H} domain and the second stabilizing domain are non-covalently associated, and wherein the second linker domain or the second stabilizing domain includes a second protease cleavage site;
b. a T Cell Receptor subunit or portion thereof selected from the group consisting of TCR-alpha, TCR-beta, CD3-gamma, CD3-epsilon, and CD3-delta, or CD4 or CD8.

68. The engineered immune cell of any one of claims 51-67, wherein one or more further protease cleavage sites are located within the first stabilization domain, the second stabilization domain, the first linker, the second linker, or the third linker.

69. An engineered immune cell comprising an engineered antigen receptor polypeptide or polypeptide complex, wherein the engineered antigen receptor polypeptide or polypeptide complex comprises an extracellular antigen-recognition polypeptide that specifically binds to a peptide or a polypeptide present in a tumor antigen, wherein the extracellular antigen-recognition polypeptide comprises an antigen binding domain, a stabilization domain, and a linker domain, wherein the stabilization domain and/or the linker domain comprise a protease cleavage site.

70. An engineered immune cell comprising an engineered antigen receptor polypeptide or polypeptide complex, wherein the engineered antigen receptor polypeptide or polypeptide complex comprises (i) a V_{HH} domain, (ii) a linker domain, (iii) a inhibitory domain, wherein the linker domain is covalently linked to the V_{HH} domain and the inhibitory domain, and wherein the linker domain and/or the inhibitory domain include a protease cleavage site, (iv) a transmembrane domain, and (v) an intracellular signaling domain.

71. An engineered immune cell comprising an engineered antigen receptor polypeptide or polypeptide complex, wherein the engineered antigen receptor polypeptide or polypeptide complex comprises: (i) a V_{HH} domain, (ii) a linker domain, (iii) a inhibitory domain, wherein the linker domain is covalently linked to the V_{HH} domain and the inhibitory domain, and wherein the linker domain and/or the inhibitory domain include a protease cleavage site, and (iv) a T Cell Receptor subunit or portion thereof selected from the group consisting of TCR-alpha, TCR-beta, CD3-gamma, CD3-epsilon, and CD3-delta, or CD4 or CD8.

72. An engineered immune cell comprising an engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, iii) a first linker domain comprising a first protease cleavage site, wherein the first linker domain is coupled to the first antibody or antigen binding domain and the masking domain, and iv) a T Cell Receptor subunit or portion thereof selected from the group consisting of TCR-alpha, TCR-beta, CD3-gamma, CD3-epsilon, and CD3-delta, or CD4 or CD8.

73. An engineered immune cell comprising an engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, iii) a first linker domain comprising a first protease cleavage site, wherein the first linker domain is coupled to the a first antibody or antigen binding domain and the masking domain, iv) a transmembrane domain, and v) an intracellular signaling domain.

74. An engineered immune cell comprising an engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, and iii) a first linker domain comprising a first protease cleavage site, wherein the first linker domain is coupled to the a first antibody or antigen binding domain and the masking domain.

75. An engineered immune cell comprising a cell surface receptor comprising:
(i) an extracellular antigen-recognition polypeptide that targets a tumor antigen, wherein the extracellular antigen-recognition polypeptide comprises a first domain comprising an activatable binding domain, a second domain comprising an inactive binding domain, and a first linker domain comprising a first protease cleavage site, wherein the first domain and the second domain are non-covalently associated whereby the second domain prevents binding of the first domain to the tumor antigen, and wherein the second domain is released from the first domain upon proteolytic cleavage at the first protease cleavage site;
(ii) a transmembrane domain; and
(iii) an intracellular signaling domain.

76. An engineered immune cell comprising a T Cell Receptor polypeptide comprising:
(i) an extracellular antigen-recognition polypeptide comprising a single chain variable fragment (scFv) domain that immunospecifically binds a tumor antigen, wherein the scFv domain comprises a first V_{H} domain, a first V_{L} domain, and at least one inactive binding domain covalently associated with the first V_{H} domain or the first V_{L} domain, optionally via a linker, and non-covalently associated with the first V_{H} domain or the first V_{L} domain, wherein the at least one inactive binding domain comprises a first protease cleavage site;
(ii) a transmembrane domain; and
(iii) an intracellular signaling domain,
wherein the T Cell Receptor polypeptide is capable of functionally interacting with a T Cell Receptor subunit selected from TCR-alpha, TCR-beta, CD3-gamma, CD3-epsilon, and CD3-delta, or CD4 or CD8.

77. An engineered immune cell comprising an engineered antigen receptor polypeptide or polypeptide complex, wherein the engineered antigen receptor polypeptide or polypeptide complex comprises an extracellular antigen-recognition polypeptide comprising:
i) a first antigen binding domain,
ii) a first linker domain comprising a first protease cleavage site,
iii) a second antigen binding domain,
iv) a second linker domain comprising a second protease cleavage site,
v) a third antigen binding domain,
vi) a third linker domain comprising a third protease cleavage site,
vii) a fourth antigen binding domain,
viii) a fifth antigen binding domain capable of binding a T cell, a TCR subunit or a CD3 delta subunit,
ix) a CD3 epsilon transmembrane domain; and
x) a CD3 epsilon intracellular signaling domain;
wherein:
a. the first linker domain is located between the first antigen binding domain and the second antigen binding domain;
b. the second linker domain is located between the second antigen binding domain and the third antigen binding domain;
c. the third linker domain is located between the third antigen binding domain and the fourth antigen binding domain; and
wherein the engineered antigen receptor polypeptide or polypeptide complex is capable of functionally associating with a T Cell Receptor complex or at least one T Cell Receptor (TCR) subunit.

78. An engineered antigen receptor polypeptide or polypeptide complex that in an activated state binds a tumor antigen, wherein the engineered antigen receptor polypeptide or polypeptide complex comprises i) a first antibody or antigen binding domain thereof that specifically binds to the tumor antigen, ii) a masking domain that inhibits the binding of the antibody or antigen binding domain thereof to the tumor antigen when associated with the antibody or antigen binding domain thereof, and iii) a first linker domain comprising a first protease cleavage site, wherein the first linker domain is coupled to the a first antibody or antigen binding domain and the masking domain.

79. An engineered immune cell comprising an engineered antigen receptor polypeptide or polypeptide complex, wherein the engineered antigen receptor polypeptide or polypeptide complex comprises
i) an extracellular antigen-recognition polypeptide that specifically binds to a peptide or a polypeptide present in a tumor antigen, wherein the extracellular antigen-recognition polypeptide comprises a first antigen binding domain, optionally a second antigen binding domain,
ii) a blocking polypeptide,
iii) a first linker domain comprising a first protease cleavage site, wherein the first linker domain is located between the extracellular antigen-recognition polypeptide and the blocking polypeptide;
iv) a transmembrane domain; and
v) an intracellular domain;
wherein the engineered antigen receptor polypeptide or polypeptide complex is capable of functionally associating with a T Cell Receptor (TCR) subunit.

80. The engineered immune cell of any one of claims 51-77 and 79, wherein the extracellular antigen-recognition polypeptide, upon cleavage of one of the protease cleavage sites has an increased binding affinity for a tumor antigen.

81. The engineered immune cell of any one of claims 51-77 and 79-80, wherein the first stabilizing domain and/or the second stabilizing domain becomes dissociated from any polypeptide with which it was non-covalently associated upon cleavage of one of the protease cleavage sites contained in the first stabilizing domain and/or in the second stabilizing domain.

82. The engineered immune cell of any one of claims 51-77 and 79-81, wherein the first stabilizing domain and/or the second stabilizing domain becomes dissociated from any polypeptide from which it was non-covalently associated upon cleavage of one of the protease cleavage sites in the linker domain, in the first stabilizing domain, and/or in the second stabilizing domain.

83. The engineered immune cell of any one of claims 51-77 and 79-82, wherein the first stabilizing domain reduces the target binding of an antigen-recognition polypeptide to which it is non-covalently associated, upon cleavage of one of the protease cleavage sites.

84. The engineered immune cell of any one of claims 51-77 and 79-83, wherein the first stabilizing domain and the second stabilizing domain are covalently associated by a third linker domain comprising a third protease cleavage site.

85. The engineered immune cell of any one of claims 51-77 and 79-84, wherein the first stabilizing domain and either the first VH domain or the second VL domain is covalently associated by a third linker domain comprising a third protease cleavage site.

86. The engineered immune cell of any one of claims 51-77 and 79-85, wherein (i) the extracellular antigen-recognition polypeptide, the transmembrane domain, and the intracellular signaling domain are derived from CD3-epsilon and (ii) the extracellular T Cell Receptor (TCR) subunit recognition polypeptide immunospecifically binds to CD3-delta.

87. The engineered immune cell of any one of claims 51-77 and 79-86, wherein (i) the extracellular antigen-recognition polypeptide, the transmembrane domain, and the intracellular signaling domain are derived from TCR-alpha and (ii) the extracellular T Cell Receptor (TCR) subunit recognition polypeptide immunospecifically binds to TCR-beta.

88. The engineered immune cell of any one of claims 51-77 and 79-87, wherein the T Cell Receptor (TCR) subunit recognition domain comprises an scFv domain or a single domain antibody.

89. The engineered immune cell of any one of claims 51-77 and 79-88, wherein the first protease cleavage site and the second protease cleavage site are susceptible to a first protease.

90. The engineered immune cell of any one of claims 51-77 and 79-89, wherein the first protease cleavage site is susceptible to a tumor-associated protease.

91. The engineered immune cell of one of claims 51-77 and 79-90, wherein the extracellular antigen-recognition polypeptide immunospecifically binds to CD 19, CD 123, CD22, CD30, CD 171, CS-1, CLL-1 (CLECL1), CD33, CD161, CD71, EGFRvDI, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC 1, EGFR, NCAM, Prosiase, PAP, ELF2M, Ephrin B2, 1GF-1 receptor, CA1X, LMP2, g 100, bcr-abl, tyrosinase, EphA2, Fucosyl GM1, sLe, GM3, TGS5, HMWMAA, o-acetyl-GD2, Folate receptor beta, TEM1/CD248, TEM7R, CLD 6, TSHR, GPRCSD, CXORF61, CD97, CD 179a, ALK, Poiysialic acid, PLAC 1, GloboH, NY-BR-1, UPK2, HAVCR1. ADRB3, PANX3, GPR20, LY6K, OR51 E2, TARP, WT1, NY-ESO-1, LAGE-1a, legumain, HPV E6, E7, MAGE-A1, ETV6-AML, sperm protein 17, XAGE1, Tie 2, MAD-CT-1, MAD-CT-2, Fos-related antigen 1, p53, p53 mutant, prostein, survivin and telornerase, PCTA-1/Galectin 8, MelanA/MART1, Ras mutant, hTERT, sarcoma translocation breakpoints, ML-IAP, ERG (TMPRSS2 ETS fusion gene), NA 17, PAX3, Androgen receptor, Cyclin B 1, MYCN, RhoC, TRP-2, CYP 1 B 1, BORIS, SART3, PAX5, OY-TES 1, LCK, AKAP-4, SSX2, RAGE-1, human telornerase reverse transcriptase, RU1, RU2, intestinal carboxyl esterase, mut hsp70-2, CD79a, CD79b, CD72, LAIR1, FCAR, LILRA2, CD300LF, CLEC 12A, BST2, EMR2, LY75, GPC3, FCRL5, AXL, IGF-1R, CD25, CD49C, gpA33, MUC-16, or IGLL1.

92. The engineered immune cell of any one of claims 51-77 and 79-91, wherein the extracellular antigen-recognition polypeptide immunospecifically binds to a stromal cell antigen selected from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In embodiments, the MDSC antigen is chosen from one or more of: CD33, CD 1 ib, C I 4, CD 15, and CD66b. Accordingly, in some embodiments, the tumor-supporting antigen is chosen from one or more of: bone marrow stromal cell antigen 2 (BST2), fibroblast activation protem (FAP) or tenascin, CD33, CD 1 lb, C14, CD 15, and CD66b.

93. The engineered immune cell of any one of claims claims 51-77 and 79-92, wherein the first protease cleavage site is susceptible to a first tumor-associated protease, and wherein the second protease cleavage site is susceptible to a second tumor-associated protease, wherein the first and second tumor-associated protease are not the same protease.

94. The engineered immune cell of any one of claims 51-77 and 79-93, wherein the extracellular antigen-recognition polypeptide is activated to target a tumor antigen upon: proteolytic cleavage of the first protease cleavage site by a first tumor associated protease, proteolytic cleavage of the second protease cleavage site by a second tumor associated protease, and proteolytic cleavage of the third protease cleavage site by a first serum protease.

95. The engineered immune cell of any one of claims 51-77 and 79-94, wherein the third protease cleavage site is susceptible to a protease present in serum.

96. The engineered immune cell of any one of claims 51-77 and 79-95, wherein the transmembrane domain and/or the intracellular signaling domain comprise a CD3 subunit sequence.

97. The engineered immune cell of any one of claims 51-77 and 79-96, wherein the first domain and the second domain are non-covalently associated whereby the second domain prevents binding of the first domain to the tumor antigen, and wherein the second domain is released from the first domain upon proteolytic cleavage at the first protease cleavage site.

98. An engineered immune cell comprising an activatable cell surface receptor polypeptide comprising:
(i) an extracellular antigen-recognition polypeptide, wherein the extracellular antigen-recognition polypeptide comprises at least a first binding pair and a second binding pair, wherein:
a. the first binding pair comprises a V_{L} domain, a first linker domain, and a first V_{H} domain, wherein the first linker domain is covalently linked to the first V_{L} domain and the first V_{H} domain, wherein the first V_{L} domain and the first V_{H} domain are non-covalently associated, and wherein the first linker domain or the first stabilizing domain includes a first protease cleavage site, and
b. the second binding pair comprising a second V_{H} domain, a second linker domain, and a second V_{L} domain wherein the second linker domain is covalently linked to the second V_{H} domain and the second V_{L} domain, wherein the second V_{H} domain and the second V_{L} domain are non-covalently associated, and wherein the second linker domain or the second stabilizing domain includes a second protease cleavage site;
(ii) a transmembrane domain; and
(iii) an intracellular signaling domain,
wherein at least one of the binding pairs recognizes and binds to a chemokine receptor protein prior to cleavage at the protease cleavage site.

99. A pharmaceutical composition comprising an engineered immune cell according to any one of claims 51-77 and 79-98 or an engineered receptor polypeptide according to claim 77.

100. A pharmaceutical composition according to claim 99, wherein the engineered antigen receptor polypeptide or polypeptide complex comprises a chimeric antigen receptor (CAR), T cell receptor (TCR) subunit, or a functional non-TCR antigen recognition receptor.

101. The pharmaceutical composition according to claim 99 or 100, wherein the first protease cleavage site is susceptible to a tumor-associated protease.

102. The pharmaceutical composition according to any one of claims 99-101, wherein the engineered antigen receptor polypeptide complex comprises a multispecific antibody.

103. The pharmaceutical composition according to any one of claims 99-102, wherein the engineered antigen receptor polypeptide complex comprises a single chain variable fragment (scFv) polypeptide capable of being directed to a target antigen, wherein the scFv polypeptide comprises a first VH domain, a first VL domain, and a first linker domain comprising a first protease cleavage site, wherein the first VH domain or the first VL domain does not specifically bind to the target antigen, wherein the target antigen is optionally present on the surface of a tumor cell.

104. The pharmaceutical composition according to any one of claims 99-103, wherein the engineered antigen receptor polypeptide complex comprises a single chain variable fragment (scFv) polypeptide capable of being directed to a target antigen, wherein the scFv polypeptide comprises a first VH domain, a first VL domain, and a first linker domain comprising a first protease cleavage site, wherein the first VH domain and the first VL domain functionally interact, and wherein the first VH domain or the first VL domain does not specifically bind to the target antigen.

105. The pharmaceutical composition according to any one of claims 99-104, wherein the engineered antigen receptor polypeptide complex comprises a single chain variable fragment (scFv) polypeptide capable of being directed to a target antigen, wherein the scFv polypeptide comprises a first VH domain, a first VL domain, and a first linker domain comprising a first protease cleavage site, wherein the first VH domain and the first VL domain functionally interact, and wherein the first VH domain or the first VL domain is inactive.

106. The pharmaceutical composition according to any one of claims 99-105, wherein the engineered antigen receptor polypeptide complex comprises a single chain variable fragment (scFv) polypeptide capable of being directed to a target antigen, wherein the scFv polypeptide comprises a first VH domain, a first VL domain, and a first linker domain comprising a first protease cleavage site, wherein the the scFv polypeptide does not specifically bind to the target antigen.

107. The pharmaceutical composition according to any one of claims 99-106, wherein the engineered antigen receptor polypeptide complex comprises a single chain variable fragment (scFv) polypeptide capable of being directed to a target antigen, wherein the scFv polypeptide comprises a first VH domain, a first VL domain, and a first linker domain comprising a first protease cleavage site, wherein the the scFv polypeptide has an affinity for the target antigen of weaker than about 50 nM.

108. The pharmaceutical composition according to any one of claims 99-107, wherein the engineered antigen receptor polypeptide complex comprises a single domain (sd) polypeptide capable of being directed to a target antigen, wherein the sd polypeptide comprises a first VH domain and a first VL domain, and a first linker domain comprising a first protease cleavage site, wherein the first VH domain and the first VL domain functionally interact, and wherein the sd polypeptide does not specifically bind to the target antigen, wherein the target antigen is optionally present on the surface of a tumor cell.

109. The pharmaceutical composition according to any one of claims 99-108, wherein the engineered antigen receptor polypeptide complex comprises i) a first receptor polypeptide comprising a first single chain variable fragment (scFv) polypeptide capable of being directed to a target antigen, wherein the first scFv polypeptide comprises a first VH domain, a first VL domain, and a first linker domain comprising a first protease cleavage site, wherein the first VH domain and the first VL domain functionally interact, wherein the first VH domain or the first VL domain does not specifically bind to the target antigen, and ii) a second receptor polypeptide second single chain variable fragment (scFv) polypeptide capable of being directed to a target antigen, wherein the second scFv polypeptide comprises a second VH domain, a second VL domain, and a second linker domain comprising a second protease cleavage site, wherein the second VH domain and the second VL domain functionally interact, wherein the second VH domain or the second VL domain does not specifically bind to the target antigen wherein the target antigen is optionally present on the surface of a tumor cell.

110. The pharmaceutical composition according to any one of claims 99-109, wherein the engineered antigen receptor polypeptide complex further comprises an intracellular signaling domain.

111. The pharmaceutical composition according to any one claims 99-110, wherein the engineered antigen receptor polypeptide complex further comprises an intracellular signaling domain comprising a signaling domain of a CD3-zeta chain polypeptide and optionally one or more additional costimulatory domains.

112. The pharmaceutical composition according to any one of claims 99-111, wherein the first receptor polypeptide and/or the second receptor polypeptide comprises an intracellular signaling domain.

113. The pharmaceutical composition according to any one of claims 99-112, wherein the engineered antigen receptor polypeptide complex further comprises a transmembrane domain.

114. The pharmaceutical composition according to any one of claims 99-113, wherein the engineered antigen receptor polypeptide complex further comprises an intracellular signaling domain and a transmembrane domain, wherein the transmembrane domain links the extracellular antigen-recognition polypeptide and the intracellular signaling domain.

115. The pharmaceutical composition according to any one of claims 99-114, wherein the engineered immune cell comprises a first genetic disruption and a second genetic disruption, wherein the first genetic disruption comprises a first disruption element encoding for the engineered antigen receptor polypeptide or polypeptide complex, and wherein the second genetic disruption comprises a second disruption element resulting in altered expression of a target gene in the engineered immune cell.

116. The pharmaceutical composition according to any one of claims 99-115, wherein the engineered immune cell comprises a T cell.

117. The pharmaceutical composition according to any one of claims 99-116, wherein the engineered antigen receptor polypeptide comprises a single chain fragment (scFv) polypeptide or a single domain (sd) polypeptide.

118. The pharmaceutical composition according to any one of claims 99-117, wherein the transmembrane domain comprises a TCR subunit transmembrane domain or portion thereof.

119. The pharmaceutical composition according to any one of claims 99-118, wherein the transmembrane domain comprises a TCR subunit intracellular signaling domain or portion thereof.

120. A nucleic acid encoding the engineered antigen receptor polypeptide or polypeptide complex of any one of claims 51-119.

121. A nucleic acid encoding a plurality of engineered antigen receptor polypeptides of any one of claims 51-120.

122. A composition comprising a plurality of nucleic acids, wherein the plurality of nucleic acids independently encode one or more engineered antigen receptor polypeptides of any one of claims 51-121.

123. A viral vector comprising the nucleic acid of claim 120 or 121.

124. A method of treatment, comprising administering the pharmaceutical composition of any one claims 99-119 to a human subject having cancer.

125. A nucleic acid encoding the activatable receptor of any of claims 1-49.

126. A viral vector comprising the nucleic acid of claim 125.
